SEQUENCE LISTING (20 sequences)

SEQ ID NO: 1 - protein, 6 aa, Artificial Sequence
  Peptide Tag Sequence
GQQQLG

SEQ ID NO: 2 - protein, 9 aa, Artificial Sequence
  Peptide Tag Sequence
GQQQLGGGG

SEQ ID NO: 3 - protein, 10 aa, Artificial Sequence
  Peptide Tag Sequence
GQQQLGGGGG

SEQ ID NO: 4 - protein, 14 aa, Artificial Sequence
  Peptide Tag Sequence
GQQQLGGGGGGGGG

SEQ ID NO: 5 - protein, 7 aa, Artificial Sequence
  Peptide Tag Sequence
WLAQRPH

SEQ ID NO: 6 - protein, 7 aa, Artificial Sequence
  Peptide Tag Sequence
PKPQQFM

SEQ ID NO: 7 - protein, 11 aa, Artificial Sequence
  Peptide Tag Sequence
GQLKHLEQQEG

SEQ ID NO: 8 - protein, 7 aa, Artificial Sequence
  Peptide Tag Sequence
PNPQLPF

SEQ ID NO: 9 - protein, 12 aa, Artificial Sequence
  Peptide Tag Sequence
NQEQVSPLTLLK

SEQ ID NO: 10 - protein, 6 aa, Artificial Sequence
  Peptide Tag Sequence
TVQQEL

SEQ ID NO: 11 - protein, 4 aa, Artificial Sequence
  Peptide Tag Sequence
QVPL

SEQ ID NO: 12 - protein, 3 aa, Artificial Sequence
  Peptide Tag Sequence
NGL

SEQ ID NO: 13 - protein, 5 aa, Artificial Sequence
  Peptide Tag Sequence
LPETG

SEQ ID NO: 14 - protein, 7 aa, Artificial Sequence
  Peptide Tag Sequence
  MISC_FEATURE (5)..(5): glycolate
AAPCXFG

SEQ ID NO: 15 - protein, 8 aa, Artificial Sequence
  Peptide Tag Sequence
  MISC_FEATURE (6)..(6): glycolate
KAAPCXFG

SEQ ID NO: 16 - protein, 690 aa, Cavia porcellus
MAEDLILERCDLQLEVNGRDHRTADLCRERLVLRRGQPFWLTLHFEGRGYEAGVDTLTFNAVTGPDPSEEAGTMARFSLSSAVEGGTWSASAVDQQDSTVSLLLSTPADAPIGLYRLSLEASTGYQGSSFVLGHFILLYNPRCPADAVYMDSDQERQEYVLTQQGFIYQGSAKFINGIPWNFGQFEDGILDICLMLLDTNPKFLKNAGQDCSRRSRPVYVGRVVSAMVNCNDDQGVLQGRWDNNYSDGVSPMSWIGSVDILRRWKDYGCQRVKYGQCWVFAAVACTVLRCLGIPTRVVTNFNSAHDQNSNLLIEYFRNESGEIEGNKSEMIWNFHCWVESWMTRPDLEPGYEGWQALDPTPQEKSEGTYCCGPVPVRAIKEGHLNVKYDAPFVFAEVNADVVNWIRQKDGSLRKSINHLVVGLKISTKSVGRDEREDITHTYKYPEGSEEEREAFVRANHLNKLATKEEAQEETGVAMRIRVGQNMTMGSDFDIFAYITNGTAESHECQLLLCARIVSYNGVLGPVCSTNDLLNLTLDPFSENSIPLHILYEKYGDYLTESNLIKVRGLLIEPAANSYVLAERDIYLENPEIKIRVLGEPKQNRKLIAEVSLKNPLPVPLLGCIFTVEGAGLTKDQKSVEVPDPVEAGEQAKVRVDLLPTEVGLHKLVVNFECDKLKAVKGYRNVIIGPA

SEQ ID NO: 17 - protein, 690 aa, Cavia cutleri; identical to SEQ ID NO: 16

SEQ ID NO: 18 - protein, 395 aa, Streptomyces mobaraensis
MHRRIHAVGQARPPPTMARGKETKSYAETYRLTADDVANINALNESAPAASSAGPSFRAPDSDDRVTPPAEPLDRMPDPYRPSYGRAETVVNNYIRKWQQVYSHRDGRKQQMTEEQREWLSYGCVGVTWVNSGQYPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRSAPGTGLVDMSRDRNIPRSPTSPGEGFVNFDYGWFGAQTEADADKTVWTHGNHYHAPNGSLGCHACLTRASSATGSEGYSDFDRGEPYVVSPSPSPRMLEHRPRQGKAGLA

SEQ ID NO: 19 - protein, 395 aa, Streptomyces mobaraensis; identical to SEQ ID NO: 18

SEQ ID NO: 20 - protein, 410 aa, Streptomyces mobaraensis
MSQRGRTLVFAALGAVMCTTALMPSAGAATGSGSGSGTGEEKRSYAETHRLTADDVDDINALNESAPAASSAGPSFRAPDSDERVTPPAEPLDRMPDPYRPSYGRAETIVNNYIRKWQQVYSHRDGRKQQMTEEQREWLSYGCVGVTWVNSGQYPTNRLAFAFFDEDKYKNELKNGRPRSGETRAEFEGRVAKDSFDEAKGFQRARDVASVMNKALENAHDEGAYLDNLKKELANGNDALRNEDARSPFYSALRNTPSFKDRNGGNHDPSKMKAVIYSKHFWSGQDRSGSSDKRKYGDPEAFRPDRGTGLVDMSRDRNIPRSPTSPGESFVNFDYGWFGAQTEADADKTVWTHGNHYHAPNGSLGAMHVYESKFRNWSDGYSDFDRGAYVVTFVPKSWNTAPDKVTQGWP

(19) United States
(12) Patent Application Publication
(10) Pub. No.: US 2025/0059499 A1
(43) Pub. Date: Feb. 20, 2025

(54) CELL-SURFACE ENGINEERING COMPOSITIONS AND METHODS FOR THE PREVENTION OF IMMUNE REJECTION

(71) Applicants: THE UNIVERSITY OF BRITISH COLUMBIA, Vancouver (CA); SIMON FRASER UNIVERSITY, Burnaby (CA)

(72) Inventors: Jayachandran N. KIZHAKKEDATHU, Vancouver (CA); Erika M.J. SIREN, Vancouver (CA); Jonathan C. CHOY, Burnaby (CA); Haiming D. LUO, Vancouver (CA); Stephen G. WITHERS, Vancouver (CA); Caigan DU, Vancouver (CA); Winnie ENNS, Burnaby (CA)

(73) Assignees: THE UNIVERSITY OF BRITISH COLUMBIA, Vancouver, BC (CA); SIMON FRASER UNIVERSITY, Burnaby, BC (CA)

(21) Appl. No.: 18/573,200

(22) PCT Filed: Jun. 22, 2022
(86) PCT No.: PCT/CA2022/051000

§ 371 Date: Dec. 21, 2023

Related U.S. Application Data
(60) Provisional application No. 63/213,322, filed on Jun. 22, 2021.

(51) Int. Cl. C12N 5/00; C07K 5/113; C07K 7/06; C07K 7/08

(52) U.S. Cl. CPC C12N 5/0006; C07K 5/1021; C07K 7/06; C07K 7/08

(57) ABSTRACT

Provided herein are compounds including a linear polyglycerol, a peptide tag, with either a linker-sugar-sialic acid moiety or a sulfate group, a preservation solutions including the compounds, methods for using the compounds or the preservation solutions to cell surface engineering (CSE) of a cell, a tissue, an organ for transplant, and methods for making the compounds. In particular, the CSE may recapitulate or rebuild glycocalyx on the luminal endothelial surface of an organ to limit immune rejection of the organ after transplant.

### CROSS REFERENCE TO RELATED APPLICATIONS

This application claims the benefit of U.S. Provisional Patent Application Ser. No. 63/213,322 filed 22 Jun. 2021 entitled “CELL-SURFACE ENGINEERING FOR THE PREVENTION OF IMMUNE REJECTION”.

### TECHNICAL FIELD

The present invention relates to localized organ engineering utilizing immune-deactivating polymers to prevent graft injury and transplant rejection. In particular, the invention relates to endothelial cell surface engineering by enzymatic ligation of functionalized linear polyglycerol polymer compounds, and protocols for use that are compatible with clinically relevant transplant preservation and perfusion conditions. Methods for making the functionalized linear polyglycerol polymer compounds, for binding such polymers to a cell surface, and for use in the engineering of organs or tissues or cells prior to transplantation in order to reduce graft injury and rejection of the organ are provided.

### BACKGROUND

Organ transplantation is a life-saving therapy for end-stage organ failure. However, immune-mediated rejection limits the curative potential of this procedure. The half-life of most transplanted organs is around 10-15 years¹. Broad acting immunosuppressive therapeutics like tracrolimus and mycophenolate mofetil that systemically inhibit immune activation are routinely employed in almost all cases to prevent rejection². However, these drugs are associated with serious adverse side effects such as increased susceptibility to infections and certain cancers, as well as post-transplant diabetes³⁻⁶. To address these adverse side effects and avoid global immune suppression, the use of controlled release of drugs⁷⁻¹⁰ and organ specific release of drugs without systemic exposure¹¹⁻¹³ have been investigated, but with limited success.

One of the major clinical challenges is the early graft inflammation caused by ischemia-reperfusion injury (IRI) resulting in delayed graft function (DGF). DGF is associated with increased risk of both acute and chronic rejection¹⁴⁻¹⁷. As such, attempts to limit IRI and resultant early graft inflammation are important. Clinical approaches to do this include limiting the production and increasing the scavenging of reactive oxygen species (ROS) as well as small-molecule or antibody-mediated global immunosuppression and anti-inflammatory strategies^{5,15, 18-20}. However, these are complicated and have proved to be sub-optimal to date.

An alternative to classical immunosuppressants is to use cell therapy to introduce immunomodulation, for example using regulatory T (T_{reg}) cells or regulatory B (B_{reg}) cells²¹⁻²³. Engineered T_{reg} and B_{reg} cells have induced immune tolerance in allografts. Conversely, combined kidney and bone marrow transplantation utilizes a transient mixed chimerism approach that enables the transplant recipient to have a mixture of both the donor's and the recipient's immune system, and has found some success in achieving persistent chimerism²⁴⁻²⁶. However, these approaches—in their current state—are sub-optimal owing to their reliance on combination therapies, the inconsistency in donor-specific tolerance induction and potential challenges in their adaptation to diverse patients.

Localized strategies applied during organs procurement and storage provide great interest and advantage to allow for treatment of the organs to occur in an isolated manner without systemic exposure to the recipient. Examples include the creation of a nano-membrane to achieve immunocloaking²⁷, but could not be adapted in current transplantation protocols. Other notable improvements to preserve organs ex vivo include promising hypothermic machine perfusion and normothermic machine perfusion, however, further refinement and research are needed before it is being used as standard preservation technique compared to static cold storage (SCS)^{28, 29}.

The endothelium is central to controlling early inflammation and damage of organ transplants as it is the first point of contact between the graft recipient and transplanted organ³⁰⁻³³. The loss of endothelial integrity caused by the breakdown of the immune-modulating endothelial glycocalyx during organ procurement and transplantation surgery generates inflammatory danger associated molecular patterns (DAMPs)^{30, 34, 35}. This in turn causes increased activation of immune cells (including macrophages, neutrophils, natural killer (NK) cells and dendritic cells), migration, infiltration, toxicity and ROS, and protease generation, which exacerbate tissue injury, edema, thrombosis and cell death^{6, 35, 36}. Current approaches to target early graft inflammation and co-stimulation, while promising, are complicated by the systemic nature of the blocking agents, which cause adverse effects elsewhere in the body that can significantly diminish patient quality of life^{4, 5, 16}. Cell surface engineering of the glycocalyx with synthetic nanoscale glycomaterials has also been tried⁸⁰.

### SUMMARY

The present invention is based, in part, on the surprising discovery that a cell-surface engineering (CSE) approach utilizing a functionalized linear polyglycerol polymer compound as described herein, may be used to minimize graft injury and graft rejection, whereby by engineering the damaged endothelial surface to rebuild the glycocalyx structure to provide long term protection or to limit immune rejection of the organ after transplant or both. The functionalized linear polyglycerol polymer compounds include a linear polyglycerol, a peptide tag, with either a linker-sugar-sialic acid moiety or a sulfate group. Furthermore, the functionalized linear polyglycerol polymer compounds described herein may be used to make preservation solutions or perfusion solutions or in CSE methods to remodel the glycocalyx on the endothelial surface of an organ or in a preservation solutions or perfusion solutions for use in CSE methods for remodeling an organ glycocalyx. The remodelling may also benefit from the addition of a cell-surface ligating enzyme.

Tissue engineering strategies that modulate the local tissue microenvironment rather than the immune response, either locally or systemically might improve current organ preservation methods by reversing existing damage to the organ, without the potential side effects of immunosuppressive therapies. Rescuing and rebuilding the functional glycocalyx barrier in a transplant organ using the functionalized linear polyglycerol polymer compounds as described herein, may prevent the release of DAMPs and the resultant activation of adaptive immune responses, thereby minimizing DGF and thus potentially prevent the rejection of the transplant organ. CSE also provides an opportunity to add functionalized linear polyglycerol polymer compounds as described herein as a way to impart immunosuppressive functional groups on the surface of graft endothelial cells to actively inhibit the immune processes. As described herein, localized CSE methods are used for immune modulation that targets the vascular endothelium with glycopolymers to impart both a cytoprotective barrier and localized immunosuppression and/or generate an immune conditioning state to protect against rejection. Described herein, are novel functionalized linear polyglycerol polymer compounds and a new therapeutic approach suitable to be applied during the current clinical procurement and preservation procedures for ex vivo transplant organs to enhance graft protection and thereby preventing transplant rejection.

In a first embodiment, there is provided a compound, the compound may have the structure of Formula A:

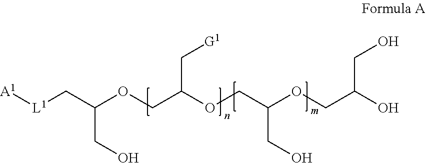

wherein, A¹ may be a peptide tag; L¹ may be a peptide linking group; G¹ may be selected from: -a linker-(a sugar)_{q1}-(a sialic acid)_{q2}; and —R¹—SO₃⁻ group; n maybe an integer between 1 and 40,000; m may be an integer between 1 and 40,000; R¹ may be selected from C, O and N; the linker may be selected from: an alkyl chain; a substituted alkyl chain; or an alkyl chain containing an azole ring; q1 may be an integer between 1 and 10; and q2 may be an integer between 1 and 10.

Alternatively, the compound may have the structure of:

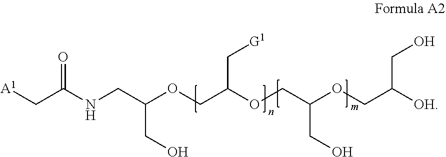

In a further embodiment, there is provided a compound, the compound may have the structure of Formula B:

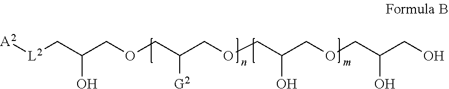

wherein, A² may be a peptide tag; L² may be a peptide linking group; G² may be selected from: -a linker-(a sugar)_{q3}-(a sialic acid)_{q4}; and a —R²—SO₃⁻ group; n may be an integer between 1 and 40,000; m may be an integer between 1 and 40,000; R² may be selected from C, O, and N; the linker may be selected from: an alkyl chain; a substituted alkyl chain; or an alkyl chain containing an azole ring; q3 may be an integer between 1 and 10; and q4 may be an integer between 1 and 10.

Alternatively, the compound may have the structure of:

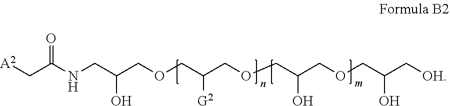

In a further embodiment, there is provided a compound, the compound comprising a hyperbranched polyglycerol (HPG) with a peptide tag, wherein the peptide tag is linked to the HPG via a peptide linking group, and wherein the HPG is further functionalized with either -linker-(a sugar)_{q5}-(a sialic acid)_{q6} groups or —R³—SO₃⁻ groups, wherein the number of groups may be an integer between 1 and 40,000, R³ may be selected from C, O, and N; the linker may be selected from: an alkyl chain; a substituted alkyl chain; or an alkyl chain containing an azole ring; q5 may be an integer between 1 and 10; and q6 may be an integer between 1 and 10.

In a further embodiment, there is provided a preservation solution, the preservation solution including a compound described herein. The preservation solution may further include a cell-surface ligating enzyme.

In a further embodiment, there is provided an ex vivo method for cell surface engineering (CSE), the method including: (a) immersing an organ in a preservation solution described herein, wherein the organ may have a glycocalyx and a vasculature; and (b) incubating the ex vivo organ in the preservation solution to permit binding of the compound a compound described herein to bind to the glycocalyx of the organ.

In a further embodiment, there is provided an ex vivo method for cell surface engineering (CSE), the method including: (a) immersing a cell in a preservation solution described herein; and (b) incubating the ex vivo cell in the preservation solution to permit binding of a compound described herein to bind to the surface of the cell.

The ex vivo method may further include a perfusing step, wherein the preservation solution may be used to perfuse the vasculature of the organ. The ex vivo method may further include a washing step.

In a further embodiment, there is provided a use of a preservation solution including a compound described herein, for organ transplantation.

In a further embodiment, there is provided a use of a preservation solution including a compound described herein for cell surface engineering of a transplant organ

In a further embodiment, there is provided a use of a preservation solution including a compound described herein, for cell transplantation.

In a further embodiment, there is provided a use of a preservation solution including a compound described herein, for cell surface engineering of a transplant cell, transplant tissue or a cell assembly.

In a further embodiment, there is provided a use of a compound described herein, for the manufacture of a preservation solution.

In a further embodiment, there is provided a method of increasing the sialic acid density on the cell or tissue or organ surface for the purpose of immunosuppression or immune conditioning post-transplantation using a compound described herein.

In a further embodiment, there is provided an ex vivo method for cell surface engineering (CSE), the method including, modifying a tissue vasculature or an organ vasculature to add sialic acid to the surface of the tissue vasculature or the organ vasculature in an amount sufficient to provide localized protection against immune mediated damage once the tissue or the organ is transplanted.

In a further embodiment, there is provided an ex vivo method for cell surface engineering (CSE), the method including, modifying a tissue vasculature or an organ vasculature to add sialic acid to the surface of the tissue vasculature or the organ vasculature in an amount sufficient to provide immune conditioning post-transplant to protect against injury or rejection.

In a further embodiment, there is provided an ex vivo method for cell surface engineering (CSE), the method including perfusing a tissue or an organ with a perfusion fluid to modify the tissue or the organ to add sialic acid to the surface of the tissue vasculature or the organ vasculature.

x¹ may be an integer between 2 and 10; x² may be an integer between 2 and 10; y² may be an integer between 2 and 10; x³ may be an integer between 2 and 10; y³ may be an integer between 2 and 10; x⁴ may be an integer between 2 and 10; y⁴ may be an integer between 2 and 10; x⁵ may be an integer between 2 and 10; and y⁵ may be an integer between 2 and 10. x¹ may be an integer between 1 and 15; x² may be an integer between 1 and 15; y² may be an integer between 1 and 15; x³ may be an integer between 1 and 15; y³ may be an integer between 1 and 15; x⁴ may be an integer between 1 and 15; y⁴ may be an integer between 1 and 15; x⁵ may be an integer between 1 and 15; and y⁵ may be an integer between 1 and 15. x¹ may be an integer between 1 and 4; x² may be an integer between 1 and 4; y² may be an integer between 1 and 4; x³ may be an integer between 1 and 4; y³ may be an integer between 1 and 4; x⁴ may be an integer between 1 and 4; y⁴ may be an integer between 1 and 4; x⁵ may be an integer between 1 and 4; and y⁵ may be an integer between 1 and 4. x¹ may be an integer between 1 and 5; x² may be an integer between 1 and 5; y² may be an integer between 1 and 5; x³ may be an integer between 1 and 5; y³ may be an integer between 1 and 5; x⁴ may be an integer between 1 and 5; y⁴ may be an integer between 1 and 5; x⁵ may be an integer between 1 and 5; and y⁵ may be an integer between 1 and 5.

n may be an integer between 1 and 40,000. m may be an integer between 1 and 40,000. Alternatively, n may be an integer between 1 and 10,000 and m may be an integer between 1 and 10,000. Alternatively, n may be an integer between 1 and 4,000 and m may be an integer between 1 and 4,000. Alternatively, n may be an integer between 1 and 5,000 and m may be an integer between 1 and 5,000. Alternatively, n may be an integer between 1 and 15,000 and m may be an integer between 1 and 15,000. Alternatively, n may be an integer between 1 and 20,000 and m may be an integer between 1 and 20,000. Alternatively, n may be an integer between 1 and 30,000 and m may be an integer between 1 and 30,000. Alternatively, n may be an integer between 1 and 50,000 and m may be an integer between 1 and 50,000. Alternatively, n may be an integer between 1 and 60,000 and m may be an integer between 1 and 60,000.

The peptide linking group may be selected from: an amide group; an alcohol group; an amine group; a thiol group; an azide group; an alkyne group; an alkene group; a carboxylic acid group; an aldehyde group; a ketone group; a halogen group; an isocyanate group; an isothiocyanate group; an oligoethylene glycol group; and a Michael acceptor/donor group.

Alternative linkers for the sugar-sialic acid linkers may be selected from: an amide; thiourea; reductive amination (via imine); hydrazone; oxime; glyoxylic-oxime; disulphide; thioether; thiazolidine; diels alder cycloaddition; CuAAC, Schiff base and Stander ligation. Alternative linkers for the sugar-sialic acid linkers may be selected from: an amide; thiourea; reductive amination (via imine); hydrazone; oxime; glyoxylic-oxime; disulphide; thioether; thiazolidine; diels alder cycloaddition; CuAAC; copper-free click chemistry agents; oligoethylene glycol; Schiff base; and Stander ligation.

The peptide tag may be selected from one or more of: GQQQLG; GQQQLGGGG; GQQQLGGGGG; GQQQLGGGGGGGGG; WLAQRPH; PKPQQFM; GQLKHLEQQEG; PNPQLPF; NQEQVSPLTLLK; TVQQEL; QVPL; QQPL; NGL; LPETG; AAPC-glycolate-FG; and KAAPC-glycolate-FG. The peptide tag may be a glutamine donor. The glutamine donor may be between 3 and 30 amino acids and includes at least one glutamine (Q). The glutamine donor may be one of more glycine (G) spacers. The peptide tag may be selected from: GQQQLG; GQQQLGGGG; GQQQLGGGGG; GQQQLGGGGGGGGG; and WLAQRPH. The peptide tag may be selected from: GQQQLG; GQQQLGGGG; GQQQLGGGGG; GQQQLGGGGGGGGG; WLAQRPH; PKPQQFM; GQLKHLEQQEG; PNPQLPF; NQEQVSPLTLLK; TVQQEL; QVPL; and QQPL. The peptide tag may be a modified peptide. The peptide tag may be selected from one or more of: GQQQLG; GQQQLGGGG; GQQQLGGGGG; and GQQQLGGGGGGGGG. The peptide tag may be GQQQLGGGG. The peptide tag may be selected from one or more of: WLAQRPH; PKPQQFM; GQLKHLEQQEG; PNPQLPF; NQEQVSPLTLLK; TVQQEL; QVPL; QQPL; NGL; LPETG; AAPC-glycolate-FG; and KAAPC-glycolate-FG. The peptide tag may be selected from one or more of: WLAQRPH; PKPQQFM; GQLKHLEQQEG; PNPQLPF; NQEQVSPLTLLK; TVQQEL; QVPL; and QQPL.

The peptide linking group may be selected from: an amide group; an alcohol group; an amine group; a thiol group; an azide group; an alkyne group; an alkene group; a carboxylic acid group; an aldehyde group; a ketone group; a halogen group; an isocyanate group; an isothiocyanate group; an oligoethylene glycol group; and a Michael acceptor/donor group. The peptide linking group may be an amide group. The peptide linking group may be selected from: an amide group; an alcohol group; an amine group; a thiol group; an azide group; an alkyne group; an alkene group; a carboxylic acid group; an aldehyde group; a ketone group; a halogen group; an isocyanate group; an isothiocyanate group; and an oligoethylene glycol group.

The sugar may be selected from: a monosaccharide; a disaccharide; and an oligosaccharide.

The sugar may be selected from: lactose; N-acetylgalactosamine (GalNac); galactose β(1-3)N-acetyllactosamine (Galβ(1-3)GalNAc); N-acetyllactosamine (LacNAc); Galβ1-4GlcNAc; Gal β1,3GlcNAc; and Gal β 1,3Glc. The sugar may be a monosaccharide. The sugar may be a disaccharide. The sugar may be an oligosaccharide. The sugar may be selected from: a monosaccharide; and a disaccharide. The sugar may be a polysaccharide. The polysaccharide may be composed of monosaccharides or disaccharides. The sugar may be selected from: lactose; N-acetylgalactosamine (GalNac); galactose ß(1-3)N-acetyllactosamine (Galß(1-3)GalNAc); N-acetyllactosamine (LacNAc); Galß1-4GlcNAc; Gal β1,3GlcNAc; and Gal β 1,3Glc.

The linker may be selected from:

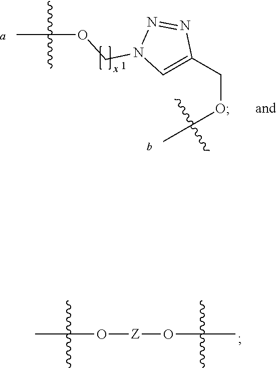

wherein, position a connects to the sugar and b position may connect to the linear polyglycerol; Z may be selected from: an alkyl chain; a substituted alkyl chain; a thioether; a disulfide; and an alkyl chain containing an azole ring.

Z may be selected from:

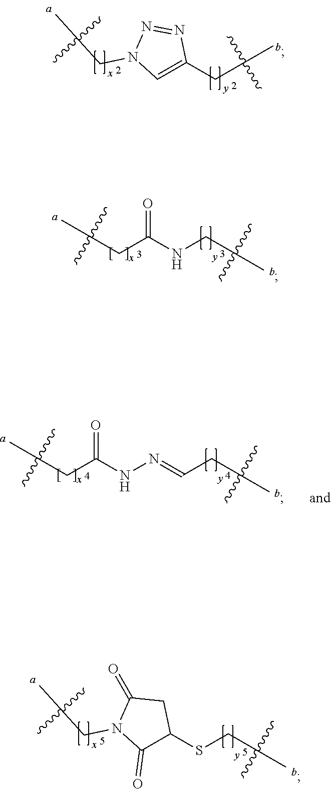

wherein, position a may connect to the sugar and b position may connect to the linear polyglycerol; x¹ may be an integer between 1 and 10; x² may be an integer between 1 and 10; y² may be an integer between 1 and 10; x³ may be an integer between 1 and 10; y³ may be an integer between 1 and 10; x⁴ may be an integer between 1 and 10; y⁴ may be an integer between 1 and 10; x⁵ may be an integer between 1 and 10; and y⁵ may be an integer between 1 and 10.

When q1 is 1, the sialic acid may be attached to the sugar by a 2,3 linkage; or 2,6-linkage; and wherein q1 is 2, the sialic acid may be attached to the sugar through a α2-3, α2-6, α2-8 linkage to the sugar or another sialic acid or sialic acid derivative.

The sugar-sialic acid may be selected from one or more of the following:

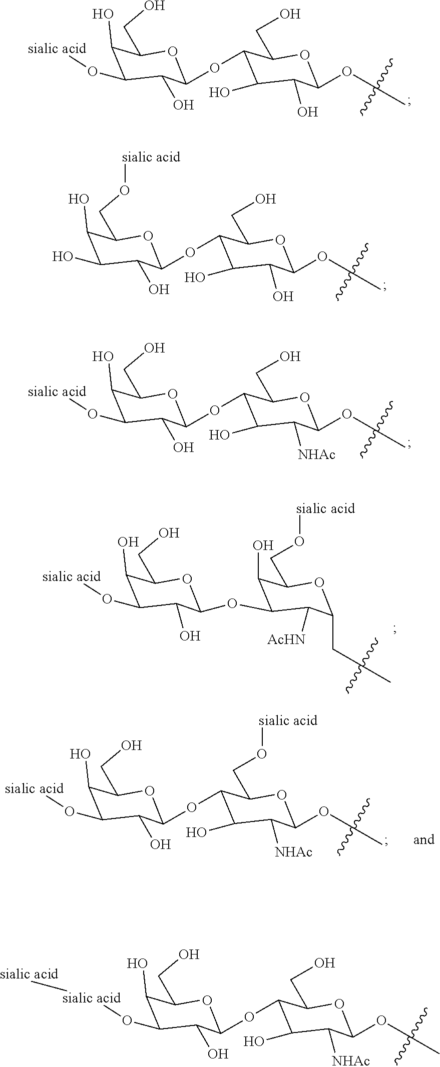

The sugar-sialic acid may be selected from one or more of the following:

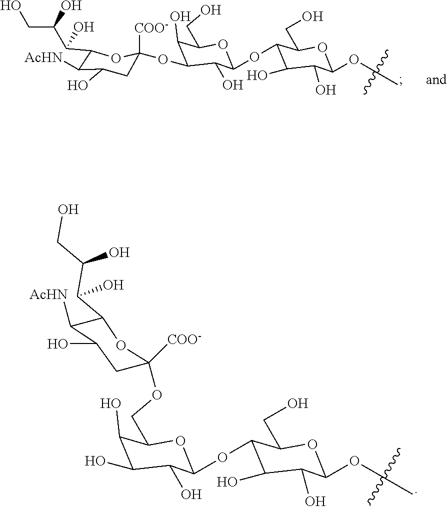

The compound may have the formula:

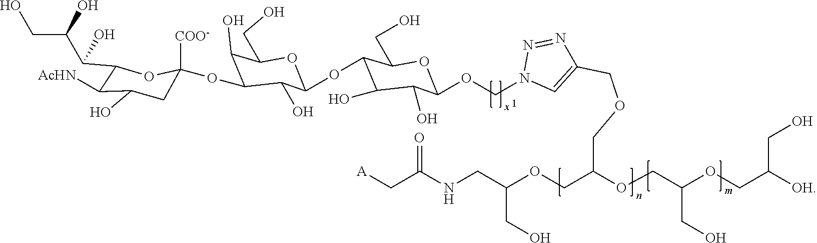

The compound may have the formula:

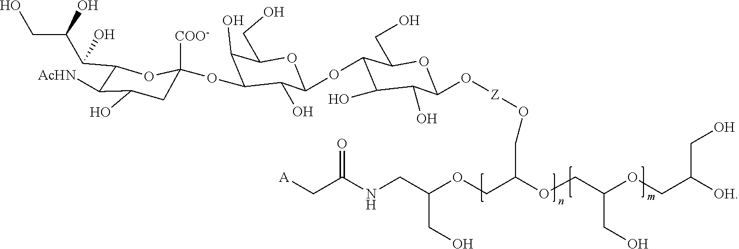

The compound may have the formula:

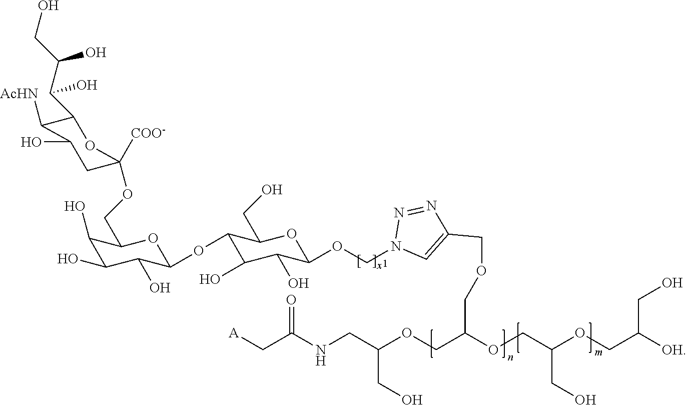

The compound may have the formula:

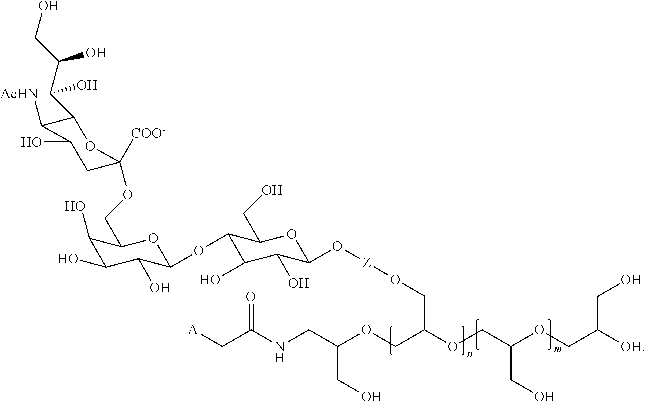

The compound may have the formula:

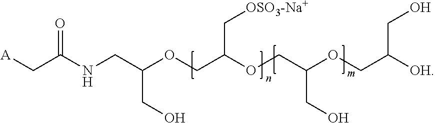

The cell-surface ligating enzyme may be a transglutaminase. The cell-surface ligating enzyme may be selected from one or more of: a transglutaminase; a sortase; an asparagine endopeptidase; a trypsin related enzyme; a butelase; and a subtiligase. The cell-surface ligating enzyme may be selected from one or more of: a transglutaminase; a sortase; an asparagine endopeptidase; a butelase; and a subtiligase. The cell-surface ligating enzyme may be selected from one or more of: a transglutaminase; a sortase; a butelase; and a subtiligase. The cell-surface ligating enzyme may be selected from one or more of: a transglutaminase; a sortase; an and a subtiligase. The cell-surface ligating enzyme may be a transglutaminase. The cell-surface ligating enzyme may be a sortase. The cell-surface ligating enzyme may be an asparagine endopeptidase. The cell-surface ligating enzyme may be a butelase. The cell-surface ligating enzyme may be a subtiligase.

The use may be for localized immunosuppression or localized inflammation prevention.

The cells may be selected from: endothelial cells, islet cells, T cells, CAR T cells, immune cells, and cultured cells. The cells may be islet cells.

The preservation solution may further include a cell-surface ligating enzyme, which may be selected from one or more of: a transglutaminase; a sortase; an asparagine endopeptidase; a trypsin related enzyme; a butelase; and a subtiligase.

The present invention is also based, in part, on the surprising discovery that graft injury and rejection may be minimized by engineering the endothelial surface of an allogenic graft via a cell-surface engineering (CSE) approach utilizing functionalized linear polyglycerol (LPG). Wherein the LPG can be functionalized with sugars, sialic acids and/or sulfates. CSE provides an opportunity to impart localized immunosuppressive functions on the surface of graft vascular cells for active inhibition of immune processes.

In some embodiments, cell-surface engineering using functionalized LPGs imparts both a cytoprotective barrier and localized immunosuppression. This new therapeutic approach may be applied during the clinical procurement and preservation procedures ex-vivo to enhance graft protection, thereby preventing the rejection without the need for heavy systemic immunosuppression. In some embodiments, the functionalized LPG is functionalized by the addition or sugar and sialic acid. In some other embodiments, the LPG is functionalized with sulfate groups which imparts localized immunosuppressive or immunomodulatory effects.

In some embodiments, of the present invention is provided a compound comprising a linear polyglycerol functionalized with a Linker, one or more sialic acids, and a Peptide Tag. Wherein the Linker may be one or more sugars, and the Peptide Tag may be a glutamine donor peptide tag. And wherein the glutamine donor peptide tag may be a Q-tag and wherein the Q tag may be the amino acid sequence GQQQLGGGG.

In some embodiments, of the present invention is provided a compound of formula 1.

(Sialic acid)y-(sugar)w-Linker-LPG-PeptideTag (Formula 1).

Wherein LPG is a linear polyglycerol. Wherein y and w can independently be 1 or 2.

Wherein the sugar may be a monosaccharide or disaccharide, and may include but not be limited to lactose, N-acetyllactosamine (GalNac), galactose ß(1-3)N-acetyllactosamine (Galß(1-3)GalNAc). Wherein the sialic acid may be attached to the sugar through a 2,3 linkage or 2,6-linkage or in the instance where y is 2, the sialic acid may be attached to the sugar through a 3′ and 8′ bond. Sialic acid may be attached as in the below Scheme

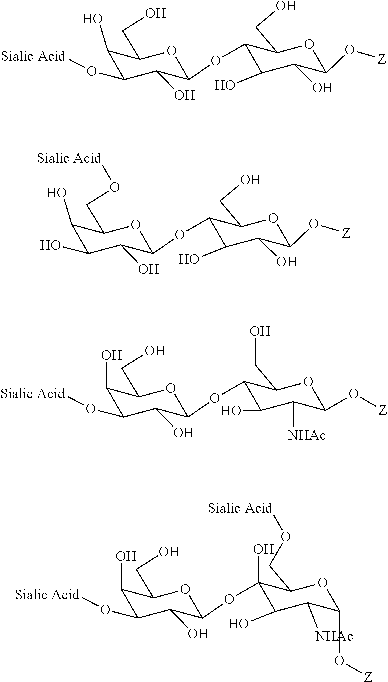

Wherein the Linker can be an alkyl chain, a substituted alkyl chain, or an alkyl chain containing an azole ring.

Wherein the PeptideTag can be a glutamine donor peptide tag or a Q-Tag.

In some embodiments, of the present invention is provided a compound of formula 2.

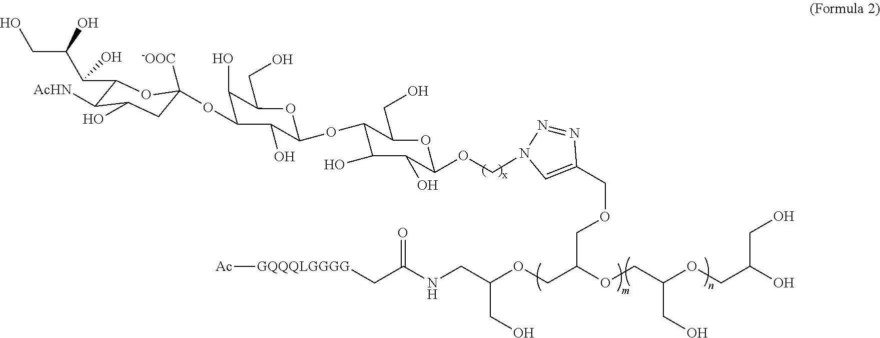

Wherein X can be 2 to 10. Wherein m and n can independently be 1-10,000.

In some embodiments, of the present invention is provided a compound of formula 3.

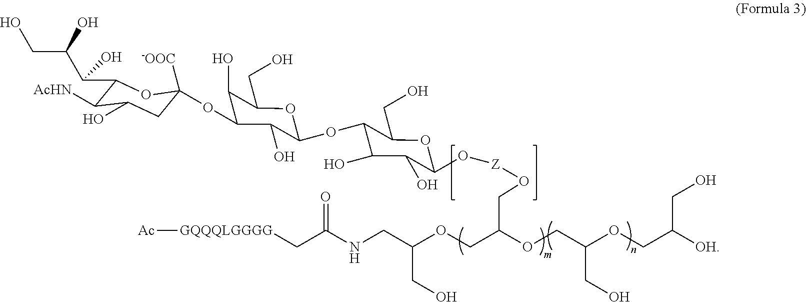

Wherein m and n can independently be 1-10,000. Wherein Z can be an alkyl chain, a substituted alkyl chain, thioether, disulfide, or an alkyl chain containing an azole ring and wherein Z may be chosen from the following groups

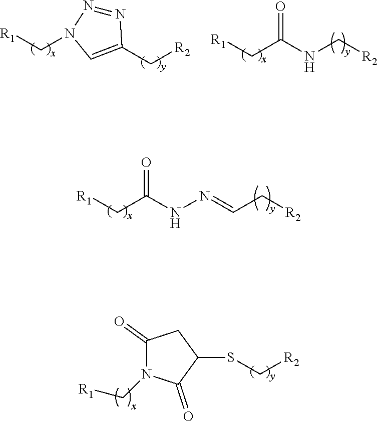

Wherein R1 is linked to the sugar and R2 is linked to LPG and x and y can independently be 2-10. Wherein m and n can independently be 1-10,000.

In some embodiments, of the present invention is provided a compound of formula 4.

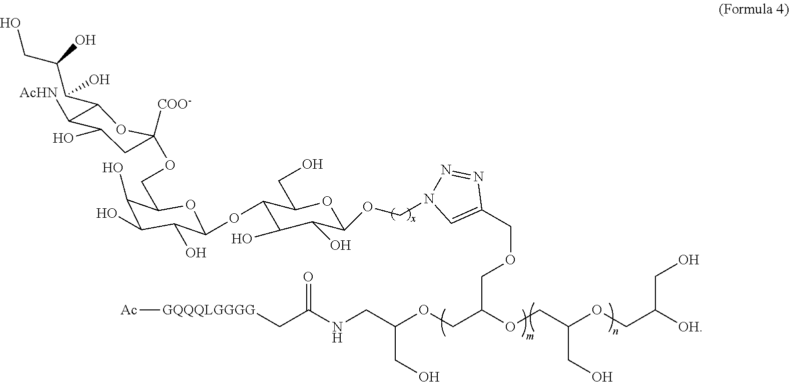

X can be 2 to 10; m and n can independently be 1-10,000.

In some embodiments, of the present invention is provided a compound of formula 5.

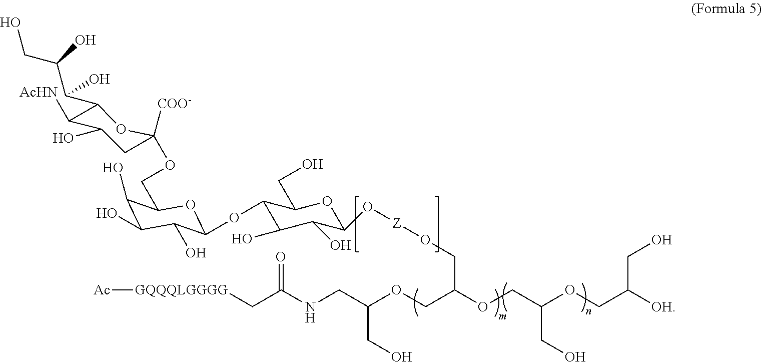

Wherein m and n can independently be 1-10,000. Wherein Z can be an alkyl chain, a substituted alkyl chain, thioether, disulfide, or an alkyl chain containing an azole ring and wherein Z may be chosen from the following groups.

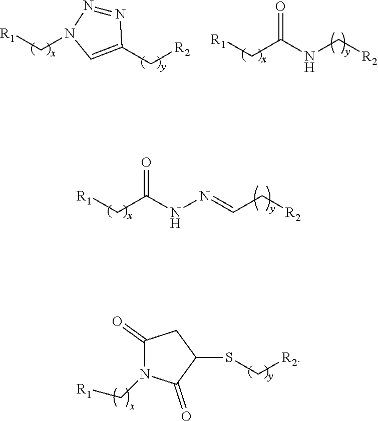

Wherein R1 is linked to the sugar and R2 is linked to LPG and x and y can independently be 2-10.

In some embodiments, of the present invention is provided a compound comprising a sulfated linear polyglycerol (LPGS) and a Peptide Tag. Wherein the Peptide Tag may be a glutamine donor peptide tag. And wherein the glutamine donor peptide tag may be a Q-tag and wherein the Q tag may be the amino acid sequence GQQQLGGGG.

In some embodiments, of the present invention is provided a compound of Formula 6 (LPGS-PeptideTag (Formula 6)).

Wherein LPGS is a sulfated linear polyglycerol.

Wherein the PeptideTag can be a glutamine donor peptide tag or a Q-Tag.

In some embodiments, of the present invention is provided a compound of formula 7.

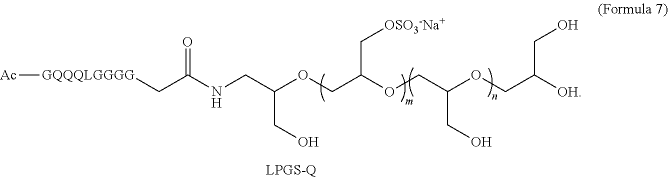

Wherein m and n can independently be 1-10,000

In some embodiments, of the present invention is provided a transplant preservation solution comprising a linear polyglycerol functionalized with a Linker, one or more sialic acids, and a Peptide Tag. Wherein the Linker may be one or more sugars, and the Peptide Tag may be a glutamine donor peptide tag. And wherein the glutamine donor peptide tag may be a Q-tag and wherein the Q tag may be the amino acid sequence GQQQLGGGG.

In some embodiments, of the present invention is provided a transplant preservation solution comprising a functionalized LPG of formulas 1, 2, 3, 4, or 5.

In some embodiments, of the present invention is provided a transplant preservation solution comprising a sulfated linear polyglycerol (LPGS) and a Peptide Tag. Wherein the Peptide Tag may be a glutamine donor peptide tag. And wherein the glutamine donor peptide tag may be a Q-tag and wherein the Q tag may be the amino acid sequence GQQQLGGGG.

In some embodiments, of the present invention is provided a transplant preservation solution comprising a compound of formula 6 or 7.

In some embodiments, of the present invention is provided a compound of formula 1, 2, 3, 4, 5, 6, or 7 as a surface immobilizing anti-inflammatory or immune modulating agent useful for organ protection.

In some embodiments, of the present invention is provided a method for reducing inflammation or immune reaction related to organ transplant, said method involving the binding a compound of formula 1, 2, 3, 4, 5, 6, or 7 to the vascular cells of organ utilizing cell-surface ligating enzyme.

In accordance with a further aspect of the invention, methods are provided for modifying the endothelial cells of an organ, the methods comprising procuring an organ and maintaining it in a transplant preservation solution comprising a compound of formula 1, 2, 3, 4, 5, 6, or 7 and then implanting said organ into a patient. In one embodiment, the organ may be from the same person. In one embodiment, the organ may be from a different person.

In accordance with a further aspect of the invention, methods are provided for modifying the endothelial cells of an organ to provide vascular protection and localized immune suppression, the methods comprising procuring an organ and maintaining it in a transplant preservation solution comprising a compound of formula 1, 2, 3, 4, 5, 6, or 7 and transplanting said organ to the patient. In one embodiment, the organ may be from the same person. In one embodiment, the organ may be from a different person. In one embodiment modifying the endothelial cells of the organ provides long lasting localized immune suppression to prevent chronic rejection.

In accordance with a further aspect of the invention is the use of compound of formula 1, 2, 3, 4, 5, 6, or 7 to modify endothelial cells provides sterically-driven immunocamouflage against leukocyte binding and/or immune inhibition at the graft surface.

In accordance with a further aspect of the invention is the use of compound of formula 1, 2, 3, 4, 5, 6, or 7 to modify nucleated cells. Said modification of nucleated cells may provide sterically-driven immunocamouflage against leukocyte binding, or immune-mediated clearance, or immune inhibition at the cell surface.

In accordance with a further aspect of the invention the use of compound of formula 1, 2, 3, 4, 5, 6, or 7 to modify organs provides antioxidant properties at the graft surface.

The transplant preservation solution can be applied during organ procurement and preservation ex-vivo under current clinical protocols. Wherein standard protocols may include static or perfusion-based organ storage conditions.

In accordance with a further aspect of the invention the linear polyglycerol is immobilized to the cell surface of an organ with a glutamine donor peptide tag. Wherein the cell may be an endothelial cell.

In accordance with a further aspect of the invention the use of compound of formula 1, 2, 3, 4, 5, 6, or 7 to modify endothelial cells may prevent intimal thickening of allograft arteries post-transplantation.

In accordance with a further aspect of the invention using an existing organ preservation/perfusion protocol with compound of formula 1, 2, 3, 4, 5, 6, or 7 could provide localized immunotherapy and may be easily translated to clinics for use on a multitude of tissue or cell types.

### BRIEF DESCRIPTION OF THE DRAWINGS

FIG. 1. Surface engineering of endothelial glycocalyx for transplant protection. (a) Proposed organ engineering showing cell surface engineering of damaged donor organ vessels during organ procurement to rebuild endothelial glycocalyx using immunosuppressive polymers. (b) General and chemical structure of cell surface reactive, glycocalyx mimicking, Q-tagged LPG-based glycopolymers used for cell surface engineering. The ball represents the polymer, the diamonds represent the glycans and Ac-GQQQLGGGG represent a Q peptide tag. (c) Synthetic overview of LPG-Q-Sia3Lac.

FIG. 2. Cell surface engineering of endothelial glycocalyx using guinea pig liver transglutaminase. (a) Polymer attachment can be carried out within organ preservation solutions in a concentration-dependent manner as assessed by flow cytometry. Ea.hy926 cells were incubated with increasing concentrations of LPG-Q (10% BODIPY FL-labelled LPG-Q) for 30 min at 4° C. in UW solution fortified with 3 mM GSH, 5 mM CaCl₂, 0.2 U/mL gtTGase and incorporation was measured by flow cytometry. (b) In the presence of gtTGase, BOPDIPY FL-labelled LPG-Q localizes to the surface of endothelial cells. Ea.hy926 cells were incubated with 1 mM LPG-Q (50% BODIPY FL-labelled LPG-Q) for 30 min at 4° C. in UW solution fortified with 3 mM GSH, 5 mM CaCl₂, 0.2 U/mL gtTGase and incorporation was visualized through confocal microscopy (z stack=0.2 μm/slice). The cell membrane is labelled with CellMask™ deep red membrane stain. Scale bars (yellow)=20 m. (c) The lifetime of BODIPY FL-labeled LPG-Q on the Ea.hy926 cells at 37° C. follows a one-phase decay profile and has a half-life of 8 hours under physiological conditions. Ea.hy926 cells were incubated with 0.5 mM LPG-Q (10% BODIPY FL-labelled LPG-Q) for 30 min at 4° C. in UW solution fortified with 3 mM GSH, 5 mM CaCl₂, 0.2 U/mL gtTGase and media was replaced with DMEM cell culture media supplemented with 10% FBS. (d) Enzyme mediated ligation occurs in the presence of cell-surface lysines, Q-tag and gtTGase. The formation of a polymer peptide conjugate has no significant effect on Q-tag reactivity. Ea.hy926 cells were incubated with PBS solution supplemented with 0.5 mM Q-tagged polymer (acyl donor), 0.2 U/mL gtTGase, 3 mM GSH, 5 mM CaCl₂ and incubated for 30 min at 4° C. (e) Enzyme mediated ligation does not induce shedding of the glycocalyx structure in Ea.hy926 cells. Ligation of LPG-Q-Sia3Lac decreases glycocalyx shedding induced by TNF-α. The glycocalyx structure was labelled using FITC labelled wheat germ agglutinin and analyzed using flow cytometry. Ea.hy926 cells were activated using TNF-α (2000 U/mL) for 3 hours at 37° C. and modified by incubating with UW solution supplemented with 0.5 mM Q-tagged polymer (acyl donor), 0.2 U/mL gtTGase, 3 mM GSH, 5 mM CaCl₂ for 30 min at 4° C. (f) Enzyme mediated ligation of HMEC-1 cell monolayers does not affect transendothelial passage. Transendothelial passage experiments were performed by tracking FITC-labelled bovine serum albumin (BSA) passage across HMEC-1 cell monolayers. HMEC-1 cells were activated using TNF-α (2000 U/mL) for 3 hours at 37° C. and modified by incubating with UW solution supplemented with 0.5 mM Q-tagged polymer (acyl donor), 0.2 U/mL gtTGase, 3 mM GSH, 5 mM CaCl₂ for 30 min at 4° C. Error bars represent 95% confidence intervals. Unpaired comparisons using a non-parametric t-test are significant with p>0.05 (ns), p≤0.05 (*), p<0.01(**), p<0.001 (***).

FIG. 3. Polymer-mediated CSE of endothelial monolayers reduces immune-cell-mediated cytotoxicity. All polymer ligations were carried out in UW solution fortified with 0.5 mM LPG-Q or 0.56 mM LPG-Q-Sia3Lac, 3 mM GSH, 5 mM CaCl₂ and 0.2 U ml-1 gtTGase for 30 min at 4° C. unless noted otherwise. (a) LPG-Q camouflages pro-inflammatory ICAM-1 receptors in a dose-dependent manner. (b) Adhesion of CFSE-labelled PBMCs to polymer-engineered endothelial cell surfaces was visualized through confocal microscopy. Scale bars, 20 μm. (c) Immunocamouflage of polymer-engineered endothelial cell surfaces attenuates the adhesion of CFSE-labelled PBMCs. (d) Both immunocamouflage (LPG-Q) and glycopolymer modification (LPG-Q-Sia3Lac and LPG-Q-Sia6Lac) suppress PBMC-mediated endothelial cell death. (e) Increasing the content of α-2,3-Sia-Lac on the base LPG polymer leads to an increase in inhibition of endothelial cell death by PBMCs. The degree of functionalization (sialylation) was measured as the percentage of hydroxyl groups per LPG scaffold (approximately 193 hydroxyl groups in total) that were converted to α-2,3-Sia-Lac. (f) Localizing immunosuppressing polymers to the cell surface (LPG-Q, LPG-Q-Sia3Lac) has a more potent effect on attenuating the PBMC-mediated cytotoxicity of endothelial cells compared to solution-based polymer immunosuppressants. Error bars represent 95% confidence intervals. Unpaired comparisons of biological replicates were performed using a non-parametric t-test. NS, not significant (P>0.05); ***P<0.001; ****P<0.0001.

FIG. 4. Polymer-mediated CSE of endothelial monolayers inhibits activation of immune cells in co-cultures. All polymer ligations were performed in UW solution fortified with 0.5 mM LPG-Q or 0.56 mM LPG-Q-Sia3Lac, 3 mM GSH, 5 mM CaCl₂ and 0.2 U ml-1 gtTGase for 30 min at 4° C. unless noted otherwise. (a) Selective depletion of CD56+CD328+ cells from PBMCs reveals that the cell-surface-engineered surfaces affected NK cells. (b)(c) Polymer-mediated glycocalyx engineering of endothelial cells inhibits CD8+ T cell activation (b) and promotes secretion of the immune suppression cytokine IL-10 (c) following co-culture with PBMCs. (d) LPG-Q-Sia3Lac modification suppresses endothelial cell death mediated by HLA-A2-reactive CAR T cells. Significant indicators on graph d are between the (−) LPG-Q and (+) LPG-Q-Sia3Lac group. Error bars represent 95% confidence intervals. Unpaired comparisons of biological replicates were performed using a non-parametric t-test, except for d, for which a one-way analysis of variance (ANOVA) was used. NS, not significant (P>0.05); ****P<0.0001.

FIG. 5. Organ engineering via polymer-based CSE of blood vessel lumen prevents inflammation and immune-mediated damage that causes vascular rejection. All polymer ligations were done in UW solution fortified with 0.5 mM LPG-Q or 0.56 mM LPG-Q-Sia3Lac, 3 mM GSH, 5 mM CaCl₂, 0.2 U/mL gtTGase for 1 hour at 4° C. Untreated (UT) and syngraft groups were treated with UW solution fortified with 3 mM GSH, 5 mM CaCl₂ and 0.2 U/mL gtTGase. (a) overview of allogenic artery transplant model. Aortic interposition grafts were performed from Balb/c donors into C57BL/6 recipients. (b) Cytokine profile of serum of recipient mice 2 days post-transplant reveals suppression of the immune system via suppression of IFN-γ signalling (left shaded area), early inflammatory cytokines (middle shaded area) and increased expression of anti-inflammatory cytokines (small right shaded area). (c) Treatment of blood vessels with LPG-Q-Sia3Lac (n=5) suppresses early inflammation 2 days post-transplant compared to UT allografts (n=5) and LPG-Q alone (n=5). Grafts were harvested at day 2 post-transplantation and cross-sections stained with H&E. Leukocyte infiltration is indicated by an arrow and areas of medial injury are indicated by an arrowhead. (d) Early graft inflammation quantified by measuring the thickness of the media. e) Semi-quantitative grading of medial inflammation. (f) Treatment of blood vessels with LPG-Q-Sia3Lac (n=8) inhibits acute rejection 15 days post-transplant compared to UT allografts (n=8). Grafts were harvested 15 days post-transplantation and cross sections were stained with H&E to reveal effects of acute rejection. Leukocyte infiltration and areas of medial injury are indicated by arrowheads (g) treatment of blood vessels with LPG-Q-Sia3Lac reduced rejection score 15 days post-transplantation. Rejection of grafts was scored on a 0-12 scale. (h) Grafts were harvested at day 42 post-transplantation and cross-sections stained with H&E. (i) Quantification of luminal narrowing in grafts. (j) Donor specific antibodies levels were quantified from sera collected at 42 days post-transplantation. Treatment of blood vessels with LPG-Q-Sia3Lac reduced donor specific antibodies in recipient mice demonstrating the initial evidence of immune conditioning to protect the transplants from injury. (k) and (l) Survival of skin grafts in artery transplanted mice. Skin from syngeneic and allogeneic (Balb/c and C3H third party) donors were grafted onto mice that received untreated and LPG-Q-Sia3Lac treated allograft blood vessels 28 days after artery transplantation. Survival of skin grafts in artery transplanted mice. (k) Balb/c and (1) C3H skin grafts reject rapidly and similarly in mice that received artery grafts treated with LPG-Q-Sia3Lac as compared to UT arteries. Error bars represents 95% confidence intervals. Unpaired comparisons using a non-parametric t-test are significant with p>0.05 (ns), p 0.05 (*), p<0.01(**), p<0.001 (***). Two-way ANOVA for multiple comparisons was used for statistical analysis of FIG. 5b, all illustrated results are statistically significant (p<0.05).

FIG. 6. Organ engineering via polymer-based CSE of blood vessel lumen imparts improved protection against ischemic mediated injury in a syngeneic renal transplant mouse model. All polymer ligation were done in UW solution fortified with 0.56 mM LPG-Q-Sia3Lac, 3 mM GSH, 5 mM CaCl₂, 0.2 U/mL gtTGase for 4 hours at 4° C. Untreated (UT) groups were treated with UW solution fortified with 3 mM GSH, 5 mM CaCl₂ and 0.2 U/mL gtTGase. (a) Overview of syngeneic renal transplant model. (b) Treatment of kidney grafts with LPG-Q-Sia3Lac imparts improved overall survival of mice (N=5) 7 days following transplantation surgery. (c) Treatment of kidney grafts with LPG-Q-Sia3Lac protects the organ by minimizing severe cellular infiltration (blue nuclear staining), tubular damage (necrosis, black arrow), and large perivascular injury (yellow arrow) restoring kidney histology to a phenotype that is similar to the sham control. (d) Treatment of kidneys with LPG-Q-Sia3Lac significantly reduces the grade of tubular damage compared to untreated (UT) controls using only UW solution. Error bars represents 95% confidence intervals. Unpaired comparisons using a non-parametric t-test are significant with p>0.05 (ns), p 0.05 (*), p<0.01(**), p<0.001(***), p<0.0001 (****). 17.0.

FIG. 7. Organ engineering via polymer-based CSE imparts improved protection against immune mediated injury in an allogenic renal transplant mouse model. All polymer ligations were done in UW solution fortified with 0.56 mM LPG-Q-Sia3Lac, 3 mM GSH, 5 mM CaCl₂, 0.2 U/mL gtTGase for 3 hours at 4° C. Untreated (UT) groups were treated with UW solution fortified with 3 mM GSH, 5 mM CaCl₂ and 0.2 U/mL gtTGase. (a) Overview of allogeneic renal transplant model, kidneys from Balb/c donors into B6 recipients. (b) Treatment of kidney grafts with LPG-Q-Sia3Lac improves survival of mice (N=8) 30 days post-transplantation. c) Blood urea nitrogen levels were improved in LPG-A-Sia3Lac treated mice. (d) kidneys were harvested 30 days post-transplantation and cross sections were stained with H&E and Masson's Trichrome. Glomerulus is indicated by light arrows, arcuate artery by black arrows and severe cell infiltration by dark arrows. Scale bars (white)=100 μm. (e) Treatment of kidneys with LPG-Q-Sia3Lac reduces cellular infiltration of renal tissue analyzed using histological scoring. (f) Mesangial expansion of the glomeruli in renal tissue was scored using Masson's Trichrome stain to reveal transplant rejection. Treatment of LPG-Q-Sia3Lac reduced mesangial expansion. Error bars represents 95% confidence intervals. Unpaired comparisons using a non-parametric t-test are significant with p>0.05 (ns), p≤0.05 (*), p<0.01(**), p<0.001(***), p<0.0001 (****).

FIG. 8. Synthetic overview of LPG-Q glycopolymers. Figure was created using ChemDraw 17.0™.

FIG. 9. LPG-Q has a higher reactivity towards the endothelial cell surface compared to LPG-SS. Ea.hy 926 cells were incubated with either LPG-Q (10% BODIPY FL-labelled LPG-Q) or LPG-SS (10% FITC-labelled LPG-SS) for 30 min at 4° C. in UW solution fortified with 3 mM GSH, 5 mM CaCl₂, 0.2 U/mL gtTGase, cells were washed and the incorporation was measured by flow cytometry. Error bars represents 95% confidence intervals.

FIG. 10. Loss of polymer attached under chemical (LPQ-SS) and enzyme (LPG-Q) ligation strategies on the endothelial cell surface following partial glycocalyx removal. Ea.hy 926 cells were incubated with either LPG-Q (10% BODIPY FL-labelled LPG-Q) or LPG-SS (10% FITC-labelled LPG-SS) for 30 min at 4° C. in UW solution fortified with 3 mM GSH, 5 mM CaCl₂, 0.2 U/mL gtTGase. Following polymer attachment, washed cells were incubated in serum starved cell media containing 0.1 mM H₂O₂ and 1 nM epinephrine for 1 hour at 37° C. Following treatment, cells were washed and analyzed by flow cytometry. The stimulation of the cells with reactive oxygen species (H₂O₂) and catecholamines (epinephrine) has been demonstrated to upregulate the expression of glycocalyx degrading extracellular proteases including human matrix metalloprotease's (MMP). Higher amount of LPG was retained on the endothelial surface when the conjugation (ligation) was performed using chemical ligation by succinimidyl succinate LPG (LPG-SS) in comparison to the enzymatic ligation using gtTGase as assessed by the amount of polymer removed from the cell surface following enzyme-mediated glycocalyx removal. The experiment provide evidence for higher glycocalyx specificity for enzymatic ligation. Error bars represents 95% confidence intervals. Unpaired comparisons using a non-parametric t-test are significant with p>0.05 (ns), p<0.05 (*), p<0.01(**), p<0.001(***).

FIG. 11. Shedding of LPG-Q into the cell culture media over time following polymer attachment. EaHy.926 cells were incubated with 0.5 mM LPG-Q (10% BODIPY FL-labelled LPG-Q) for 30 min at 4° C. in UW solution fortified with 3 mM GSH, 5 mM CaCl₂, 0.2 U/mL gtTGase and media was replaced with phenol-free DMEM cell culture media supplemented with 10% FBS at 37° C. and 5% CO₂. Normalized fluorescent intensity (λₑₓ/λₑₘ=503/520 nm) of the cell media was collected at various time points and normalized to cell media collected from non-polymer labelled cells collected at the same time point.

FIG. 12. Assessment of enzyme-mediated polymer attachment in unactivated and TNF-α activated Ea.hy 926 endothelial cells as quantified by ammonia (NH₃) generation. Ea.hy 926 cells were activated with TNF-α (2000 U/mL, 3 hours) in serum deficient DMEM and then incubated with 0.5 mM LPG-Q for 30 min at 4° C. in UW solution fortified with 3 mM GSH, 5 mM CaCl₂ and 0.2 U/mL gtTGase. The supernatant was collected and assays for NH₃ content. Error bars represents 95% confidence intervals.

FIG. 13. Assessment of enzyme-mediated polymer attachment in HMEC-1 endothelial cells as quantified by ammonia (NH₃) generation. HMEC-1 cells were incubated with 0.5 mM LPG-Q for 30 min at 4° C. in UW solution fortified with 3 mM GSH, 5 mM CaCl₂ and 0.2 U/mL gtTGase. The supernatant was collected and assays for NH₃ content. Unpaired comparisons using a non-parametric t-test are significant with p>0.05 (ns), p<0.05 (*), p<0.01(**), p 0.001 (***).

FIG. 14. Assessment of enzyme-mediated polymer attachment in HUVEC endothelial cells as quantified by ammonia (NH₃) generation. HUVEC cells were incubated with 0.5 mM LPG-Q for 30 min at 4° C. in UW solution fortified with 3 mM GSH, 5 mM CaCl₂ and 0.2 U/mL gtTGase. The supernatant was collected and assays for NH₃ content. Unpaired comparisons using a non-parametric t-test are significant with p>0.05 (ns).

FIG. 15: Comparative immunocamouflage of a similar number of LPG-Q or LPG-Q-Sia3Lac polymers grafted to Ea.hy 926 endothelial surfaces. Ea.hy 926 cells were activated with TNF-α (2000 U/mL, 3 hours) in serum deficient DMEM and then incubated with 0.5 mM LPQ-Q or 0.56 mM LPG-Q-Sia3Lac for 30 min at 4° C. in UW solution fortified with 3 mM GSH, 5 mM CaCl₂ and 0.2 U/mL gtTGase. The cells were washed and then labelled with PE-Cy™5 Mouse Anti-Human CD54 and assayed using flow cytometry. Error bars represents 95% confidence intervals.

FIG. 16: Comparative endothelial toxicity of TNF-α activated Ea.hy 926 endothelial cells cultured with IL-2 activated PBMCs that have been depleted of various immune cell populations. Ea.hy 926 cells were activated with TNF-α (2000 U/mL, 3 hours) in serum deficient DMEM and then incubated with 0.5 mM LPG-Q for 30 min at 4° C. in UW solution fortified with 3 mM GSH, 5 mM CaCl₂ and 0.2 U/mL gtTGase. PBMCs were cultured for 24 h in RPMI-1640 containing IL-2 (1000 U/mL). After cell depletion, immune cells were co-incubated with endothelial cells at an effector to target ratio of 10:1 for 18 hours and cytotoxicity was evaluated by an LDH assay. Error bars represents 95% confidence intervals. Unpaired comparisons using a non-parametric t-test are significant with p>0.05 (ns), p<0.05 (*), p<0.01(**), p<0.001 (***).

FIG. 17: Assessment of IL-6 released into the cell media following PBMC/endothelial co-culture. All polymer attachment was done using 0.56 mM LPG-Q-Sia3Lac for 30 min at 4° C. in UW solution fortified with 3 mM GSH, 5 mM CaCl₂ and 0.2 U/mL gtTGase. Following attachment, endothelial cells were co-cultured with IL-2 activated PBMCs for 18 hours and IL-6 secretion was measured by ELISA. Error bars represents 95% confidence intervals.

FIG. 18: RP-HPLC chromatogram of Q tag as analyzed by CanPeptide Inc.™ Trityl (trt) protected peptide, Ac-GQ(trt)Q(trt)Q(trt)LGGGGG-OH, purity was assessed to be 83.28% pure as shown in peak eluted at 25.829 minutes. A Waters 600 Multi-Solvent System™ using a Phenomenex Jupiter Proteo™ (C18, 4u, 4.6×250 mm) column. The mobile phase is composed of 0.1% trifluoro acetic acid in acetonitrile.

FIG. 19: Electrospray ionization mass spectrometry (ESI-MS) chromatogram of Q-tag as analyzed by CanPeptide Inc. Trityl (trt) protected peptide, Ac-GQ(trt)Q(trt)Q(trt)LGGGGG-OH, identity was confirmed as shown in peak 1627.7828 ([M+1]).

FIG. 20: ¹H NMR of ethoxy ethyl glycidyl ether (EEGE) monomer in CDCl₃.

FIG. 21: ¹H NMR analysis of pEEGE-N₃ in CDCl₃.

FIG. 22: ¹H NMR analysis of LPG-N₃ in D₂O (Mₙ=14900 Da).

FIG. 23: GPC chromatogram of the unfunctionalized LPG-N₃ scaffold.

FIG. 24: ¹H NMR analysis of propargylated LPG-N₃ in MeOD.

FIG. 25: Representative FTIR characterization of LPG-N₃ before (grey) and after (black) treatment with PPh₃. FTIR spectra were recorded on a Bruker TENSOR II FTIR spectrometer with a resolution of 4 cm⁻¹. The sharp absorption band at 2100 cm⁻¹ in LPG-N₃ is characteristic of asymmetric stretching of the azido (—N₃) group (see arrow). The polymer samples were analyzed using the KBr pellet method. FTIR spectra were obtained by scanning 64 times at atmospheric conditions. The OPUS™ spectroscopic software was used for data handling.

FIG. 26: ¹H NMR analysis of propargylated LPG-Q in MeOD. The multiplet between 0.91-0.89 ppm represent the leucine methyl groups of the Q-tag (Ac-GQQQLQQQQQ). The amount of peptide per LPG molecule is ˜1.0.

FIG. 27: Representative 1H NMR analysis of LPG-Q-Sia3Lac in D₂O. The separated peaks at 1.99 ppm refer to the N-acetyl group of the α2,3-Sialylactose substrate. The calculated degree of functionalization from NMR is ˜9.0%(17 groups).

FIG. 28: Representative 1H NMR analysis of LPG-Q-Sia6Lac in D₂O. The separated peaks at 1.99 ppm refer to the N-acetyl group of the α2,6-Sialylactose substrate. The calculated degree of functionalization from NMR is ˜9.0% (16 groups).

FIG. 29: Representative ¹H NMR analysis of LPG-Q-Lac in D₂O. The multiplet from 1.54-1.26 ppm refer to the 8 internal protons of the C6 linker on the lactose substrate. The calculated degree of functionalization from NMR is ˜8.0%(15 groups).

FIG. 30: ¹H NMR analysis of LPG-SS in D₂O.

FIG. 31: NH₄Cl standard curve generated using ammonia assay kit (Sigma™, MAK310). The fluorescence intensity was measured at λₑₓ=360 nm/λₑₘ=450 nm using a spectrophotometer. The slope of the standard curve is used to calculate the concentration of NH₃ generated using Eq. 1.

FIG. 32: Representative flow cytometry profiles to validate the removal of various immune cells from the PBMC milieu. (A) CD45+CD328+(Siglec-7) NK cells (Q1-UR) before (top) and after depletion. (B) Dot plots of PBMC milieu before (top) and after (bottom) monocyte depletion. Monocytes have forward scattering (FSC, x-axis) and side-scattering (SSC, y-axis) that is unique from other PBMCs. With this, no identifying fluorescent marker was used). (C) Population distribution of cells within the ‘lymphocyte’ gate of the dot plot in (B) that have been stained with CD8+ mAb before (top) and after (bottom) CD8+ T-cell depletion.

FIG. 33: Flow Cytometry profile to validate the expression of HLA-A2 antigen on native Ea.hy926 cells (light grey) and TNF-α-stimulated Ea.hy926 cells (dark grey) using anti-HLA-A2. Ea.hy926 cells were activated using TNF-α (2000 U/mL for 3 hours).

FIG. 34: Representative images of skin grafts transplantation. Skin from syngeneic (C57BL/6), allogeneic donor (Balb/c) and allogeneic third party donor (C3H) were grafted onto recipients of either untreated or LPG-Q-Sia3Lac treated artery allografts 28 days after artery transplantation.

FIG. 35: Representative figures showing scoring mesangial expansion of the glomeruli. 1 (o %-24% of the area affected with densely stain), 2 (25%-49%), 3 (50%-74%), and 4 (>75%).

FIG. 36: ¹H NMR of ethoxy glycidyl ether monomer in CDCl₃.

FIG. 37: ¹H NMR of poly(ethoxy glycidyl ether) in CDCl₃.

FIG. 38: Representative ¹H NMR of α-amino linear polyglycerol (LPG-NH₃) in D₂O.

FIG. 39: GPC chromatogram of α-amino linear polyglycerol (LPG-NH₃).

FIG. 40: Representative 1H NMR of LPG-Q in MeOD. The multiplet around 0.86 ppm represent the leucine methyl groups of the glutamine peptide.

FIG. 41: Representative 1H NMR of LPGS-Q in MeOD. The multiplet around 0.86 ppm represent the leucine methyl groups of the glutamine peptide.

FIG. 42: tTgase-mediated bioconjugation with endothelial cells. (a) Bioconjugation was monitored by assessing NH₃ generation. (b) Viability with endothelial EAhy.926 cells using tTGase-mediated bioconjugation compatibility of polymers attached on the cell surface (+tTGase) and in solution (−tTGase). Unpaired comparisons using a non-parametric t-test are significant with p>0.05 (ns), p<0.05(*).

FIG. 43: Superoxide anion scavenging activity assay. (a) Superoxide anion scavenging ability of various polymers in solution. (b) concentration dependent superoxide anion scavenging ability of polyglycerols in solution.

FIG. 44: Superoxide anion scavenging activity under in vitro conditions with EA.hy 926 cells. (a) Scavenging activity of cells with varying growth incubation periods at similar seeding densities. (b) Cell viability under oxidative stress in the presence of various polymers in solution.

FIG. 45: Scavenging superoxide anion with LPGS covalently bound on cell surfaces—(a) Superoxide anion scavenging properties of cell with and without polymers. (b) 0′₂ scavenging activity of surface modified cells with various concentration of LPGS. Unpaired comparisons using a non-parametric t-test are significant with p>0.05 (ns), p<0.05(*), p<0.01 (**), p<0.001(***).

FIG. 46: PMS-induced glycocalyx shedding of EA.hy 926 monolayers through oxidative stress. (a) The glycocalyx was labelled using WGA stain and analyzed using flow cytometry using various concentrations of PMS. (b) EA.hy 926 monolayers were modified with various polymers to assess their ability to prevent glycocalyx shedding. Unpaired comparisons using a non-parametric t-test are significant with p>0.05 (ns), p<0.05(*), p<0.01 (**), p<0.001(***).

FIG. 47: PBMC adhesion (a) and PBMC-mediated cytotoxicity (b) on TNF-α activated and surface modified EA.hy 926 monolayers. PBMCs were activated using IL-2. Unpaired comparisons using a non-parametric t-test are significant with p>0.05 (ns), p<0.05(*), p<0.01 (**), p<0.001(***).

FIG. 48: Trans-endothelial membrane function of modified monolayers. Transendothelial passage experiments were performed by tracking FITC-labelled bovine serum albumin (BSA) passage across HMEC-1 cell monolayers. HMEC-1 cells were activated using TNF-α (2000 U/mL) for 3 hours at 37° C. and modified by incubating with UW solution supplemented with 0.5 mM polymer, 0.2 U/mL gtTGase, 3 mM GSH, 5 mM CaCl₂ for 30 min at 4° C.

FIG. 49: Polymer-mediated cell surface engineering of endothelial monolayers inhibits activation of immune cell-mediated cytotoxicity in co-cultures. Surface modification suppresses endothelial cell death mediated by HLA-A2 reactive CAR-T cells.

FIG. 50: ELISA of TNF-α release by M1-like macrophages (pro-inflammatory state). Various polymers were co-incubated to reveal their anti-inflammatory properties indicated by the suppressed release of the inflammatory cytokine, TNF-α. Error bars represent 95% confidence intervals. Unpaired comparisons using one way ANOVA tests are significant with p>0.05 (ns), p≤0.05 (*), p<0.01(**), p<0.001(***).

FIG. 51: Cytokine panel 2 days post-transplantation. All polymer ligations were performed in UW solution fortified with 0.5 mM LPGS-Q, 3 mM GSH, 5 mM CaCl₂ and 0.2 U/mL gtTGase for 1 h at 4° C. Untreated control groups were treated with UW solution fortified with 3 mM GSH, 5 mM CaCl₂ and 0.2 U/mL gtTGase. Allogeneic aortic interposition grafts were performed from BALB/c donor mice into C57/BL/6 recipient mice. Cytokine profile of the sera of recipient mice 2 days post-transplant.

FIG. 52: Cytokine panel 15 days post-transplantation. All polymer ligations were performed in UW solution fortified with 0.5 mM LPGS-Q, 3 mM GSH, 5 mM CaCl₂ and 0.2 U/mL gtTGase for 1 h at 4° C. Untreated control groups were treated with UW solution fortified with 3 mM GSH, 5 mM CaCl₂ and 0.2 U/mL gtTGase. Allogeneic aortic interposition grafts were performed from BALB/c donor mice into C57/BL/6 recipient mice. Cytokine profile of the sera of recipient mice 15 days post-transplant.

FIG. 53: Cell surface engineering of various cell glycocalyx using gtTGase. Cells were incubated with 0.5 mM Q-tagged polymer, 0.2 U ml-1 gtTGase, 3 mM GSH and 5 mM CaCl₂ in various cell surfaces. Conjugation efficiency was assessed by NH₃ production which is a bi-product in the transglutamination reaction when using gtTGase.

FIG. 54: Cell surface engineering of endothelial glycocalyx using gtTGase. EA.hy926 cells were incubated with PBS solution supplemented with 0.5 mM Q-tagged polymer, 0.2 U ml-1 gtTGase, 3 mM GSH and 5 mM CaCl₂ in various temperatures and incubation lengths. Conjugation efficiency was assessed by NH₃ production which is a bi-product in the transglutamination reaction when using gtTGase.

FIG. 55: Cell surface engineering of endothelial glycocalyx using gtTGase. EA.hy926 cells were incubated with PBS solution supplemented with 0.5 mM Q-tagged polymer, 0.2 U ml-1 gtTGase, 3 mM GSH and 5 mM CaCl₂ in various temperatures and incubation lengths. Cell viability was assessed using an MTS assay.

FIG. 56: Modifying platelet surface with peptide and polymers at 4° C. Platelet surface modification did not induce platelet activation as assessed by flow cytometry using CD62 surface markers. TRAP was used as a positive control for platelet activation.

FIG. 57: Compatibility of cell surface engineering on human pancreatic islet surfaces using gtTGase. Human pancreatic islets were incubated with 0.5 mM Q-tagged polymer, 0.2 U ml-1 gtTGase, 3 mM GSH and 5 mM CaCl₂ at room temperature for 30 minutes. Cell viability was assessed using a LIVE/DEAD stain assay and insulin producing activity was assessed by DTZ detection assay.

FIG. 58: Cell surface engineering of endothelial glycocalyx using TGase isoforms. EA.hy926 cells were incubated with 0.5 mM Q-tagged polymer, 0.2 U ml-1 TGase, 3 mM GSH and 5 mM CaCl₂ at 4° C. for 30 minutes. TGase from guinea pig liver (gtTGase) and microbial origin (mTGase) were tested for the efficacy and compatibility. polymers used for gtTGase is GQQQLGGGGG-PEG and WLAQRPH-PEG. Conjugation efficiency was assessed by NH₃ production which is a bi-product in the transglutamination reaction when using TGase. Cell viability was assessed using an MTS assay.

### DETAILED DESCRIPTION

The following detailed description will be better understood when read in conjunction with the appended figures. For the purpose of illustrating the invention, the figures demonstrate embodiments of the present invention. However, the invention is not limited to the precise arrangements, examples, and instrumentalities shown.

Any terms not directly defined herein shall be understood to have the meanings commonly associated with them as understood within the art of the invention.

The term “linear polyglycerol” or LPG is used herein as it is normally understood to a person of ordinary skill in the art and often refers to a polyether-polyol or a polyglycerol having no degree of branching. The LPG may be 1,2-linked linear polyglycerol (as shown in Formula A), but may also be a 1,3-linked linear polyglycerol (as shown in Formula B).

The term “sulfated linear polyglycerol” or LPGS is used herein as it is normally understood to a person of ordinary skill in the art and often refers to a polyether-polyol or a polyglycerol having one or more sulfates bound. As used herein, “sulfonated” would include having a R—SO₃⁻ group, wherein the R may be 0, N or C (with or without the counter ion (+)) and “sulfated” refers to the addition of a SO₄²⁻ moiety (with or without the counter ions (2+)). The degree of sulfonation may be in the range of between 0.1 and 99.99%. There may be a benefit to having the negatively charged when functionalizing a linear polyglycerol. Alternatively, another negatively charged groups similar to sulfate groups might be present at this position. Alternative groups may be carboxylate and phosphate groups.

The term “linker” is used herein to be a linking moiety between the LPG and the sugar(s)/sialic acid(s). The linker may be an alkyl chain; a substituted alkyl chain; or an alkyl chain containing an azole ring. Substitutions of the alkyl may be N, S or O as carbon substitutions or CH₃, CH₂CH₃, F, Cl, Br, CF₃, OCF₃, OCF₂, S(═O)₂(NH₂) or OCH₂C(CH₃)(CH₂) as hydrogen substitutions. The “alkyl” in the alkyl chain or substituted alkyl chain or an alkyl chain containing an azole ring, may have anywhere between 2 and 10 carbons. Alternatively, the alkyl chain may have 1 to 10 carbons, or 1 to 15 carbons, 2 to 3 carbons, 1 to 3 carbons, 2 to 4 carbons, 1 to 4 carbons, 2 to 5 carbons, 1 to 5 carbons, 2 to 6 carbons, 1 to 6 carbons, 2 to 7 carbons, 1 to 7 carbons, 2 to 8 carbons, 1 to 8 carbons, 2 to 9 carbons, 1 to 9 carbons, 2 to 11 carbons, 1 to 11 carbons, 2 to 12 carbons, 1 to 12 carbons, 2 to 13 carbons, 1 to 13 carbons, 2 to 14 carbons, 1 to 14 carbons, 2 to 15 carbons, 1 to 16 carbons, 2 to 16 carbons, 1 to 17 carbons, 2 to 17 carbons, 1 to 18 carbons, 2 to 18 carbons, 1 to 19 carbons, 2 to 19 carbons, 1 to 20 carbons, 2 to 20.

Alternative linkers for the sugar-sialic acid linkers may be selected from: an amide; thiourea; reductive amination (via imine); hydrazone; oxime; glyoxylic-oxime; disulphide; thioether; thiazolidine; diels alder cycloaddition; CuAAC; copper-free click chemistry agents; oligoethylene glycol; Schiff base; and Stander ligation.

The linker may be selected from:

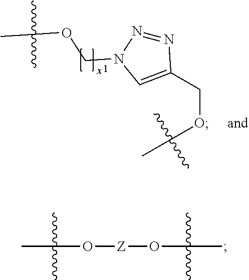

wherein Z may be selected from: an alkyl chain; a substituted alkyl chain; a thioether; a disulfide; and an alkyl chain containing an azole ring. Alternatively, Z may be selected from:

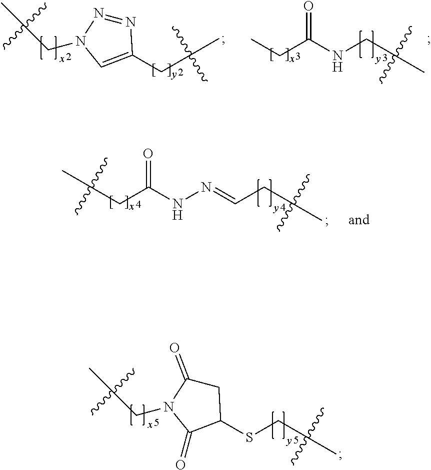

wherein x¹ may be an integer between 2 and 10; x² may be an integer between 2 and 10; y² may be an integer between 2 and 10; x³ may be an integer between 2 and 10; y³ may be an integer between 2 and 10; x⁴ may be an integer between 2 and 10; y⁴ may be an integer between 2 and 10; x⁵ may be an integer between 2 and 10; and y⁵ may be an integer between 2 and 10.

The term “sugar” as used herein refers to a soluble carbohydrate molecule and as used herein may include any one or more monosaccharides or any one or more disaccharides or an oligosaccharide. For example, sugars may include monosaccharides, disaccharides, lactose, N-acetylgalactosamine (GalNac), and galactose β(1-3)N-acetyllactosamine (Galβ(1-3)GalNAc). Sugars may include disaccharides, lactose, N-acetylgalactosamine (GalNac), galactose β(1-3)N-acetyllactosamine (Galβ(1-3)GalNAc). Alternatively, sugars may include lactose; N-acetylgalactosamine (GalNac); galactose β(1-3)N-acetyllactosamine (Galβ(1-3)GalNAc); N-acetyllactosamine (LacNAc); Galβ1-4GlcNAc; Gal β1,3GlcNAc; and Gal β1,3Glc. Alternatively, sugars may include lactose, N-acetyllactosamine (GalNac), or galactose β(1-3)N-acetyllactosamine (Galβ(1-3)GalNAc). Alternatively, sugars may include disaccharides, lactose, N-acetylgalactosamine (GalNac), and galactose β(1-3)N-acetyllactosamine (Galβ(1-3)GalNAc). Alternative sugars may include glucose, fructose, galactose, sucrose (glucose+fructose), lactose (glucose+galactose), and maltose (two molecules of glucose).

Where there is one sugar a sialic acid may be attached to the sugar by a 2,3 linkage; or 2,6-linkage. Alternatively, where there are 2 sugars, the sialic acid may be attached to the sugar through a α2-3, α2-6, α2-8 linkage to the sugar or another sialic acid or sialic acid derivative. Alternatively, a 2,3 linkage; or 2,6-linkage; and wherein q2 is 2, the sialic acid or sialic acid derivative may be attached to the sugar through a 3′ and 8′ linkage.

The term “peptide tag” is used herein refers to a peptide having a sequence of 3-30 amino acids attached to a terminus of the linear polyglycerol compounds as described herein. In particular, the peptide tag may be linked to the polyglycerol via a peptide linking group, that may be selected from one or more of: an amide group; an alcohol group; an amine group; a thiol group; an azide group; an alkyne group; an alkene group; a carboxylic acid group; an aldehyde group; a ketone group; a halogen group; an isocyanate group; an isothiocyanate group; oligoethylene glycol spacer; and a Michael acceptor/donor group. The peptide tags described herein may have a sequence suitable for binding to a transplant organ surface, a tissue surface or a cell surface. In particular, the peptide tag may be compatible with one or more cell-surface ligating enzymes to facilitate the linking of the functionalized linear polyglycerol polymer compounds to the endothelial surface of a transplant organ, tissue or cell, in particular the endothelial glycocalyx of a transplant organ or the cell surface of an islet cell for transplant or glycocalyx of an immune cell. Once bound to the transplant organ surface, tissue or cell via the peptide tag, the functionalized linear polyglycerol polymer compounds may create an endothelial glycocalyx on the endothelial surface of an organ, or on the surface of a tissue or cell to limit immune rejection of the after transplant. In particular, a suitable peptide tag may be a glutamine donor peptide tag or a Q-tag. Furthermore, the peptide tag may be an alternative TGase peptide (i.e. PKPQQFM; GQLKHLEQQEG; PNPQLPF; NQEQVSPLTLLK; TVQQEL; QVPL; and QQPL). Alternatively, the peptide tag may be selected from one or more of the following: NGL (for Oldenlandia affinis asparaginyl endopeptidase 1 (OaAEP-1); LPETG (for sortase); AAPC-glycolate-FG (for subtiligase); and KAAPC-glycolate-FG (for subtiligase).

The term “glutamine donor peptide tag” is used herein as it is normally understood to a person of ordinary skill in the art and often refers to a type of peptide tag having a sequence of 3-30 amino acids including glutamine. It can also contain G1 or G5 glycine spacer. This includes a Q tag and can be for example, Ac-GQQQLG-OH, Ac-GQQQLGGGGG-OH, AcGQQQLGGGGGGGGG and Ac-WLAQRPH-OH. The glutamine donor peptide tag may be selected from one or more of: GQQQLG; GQQQLGGGG; GQQQLGGGGG; GQQQLGGGGGGGGG; and WLAQRPH. The glutamine donor peptide tag may be selected from one or more of: GQQQLG; GQQQLGGGG; GQQQLGGGGG; GQQQLGGGGGGGGG; WLAQRPH; PKPQQFM; GQLKHLEQQEG; PNPQLPF; NQEQVSPLTLLK; TVQQEL; QVPL; and QQPL.

The peptide tag may be selected from one or more of: GQQQLG; GQQQLGGGG; GQQQLGGGGG; GQQQLGGGGGGGGG; WLAQRPH; PKPQQFM; GQLKHLEQQEG; PNPQLPF; NQEQVSPLTLLK; TVQQEL; QVPL; QQPL; NGL; LPETG; AAPC-glycolate-FG; and KAAPC-glycolate-FG.

TABLE 1
Exemplary Peptide-Tag Sequences

| Peptide Sequence | SEQ ID NO |
|---|---|
| GQQQLG | 1 |
| GQQQLGGGG | 2 |
| GQQQLGGGGG | 3 |
| GQQQLGGGGGGGGG | 4 |
| WLAQRPH | 5 |
| PKPQQFM | 6 |
| GQLKHLEQQEG | 7 |
| PNPQLPF | 8 |
| NQEQVSPLTLLK | 9 |
| TVQQEL | 10 |
| QVPL | 11 |
| NGL | 12 |
| LPETG | 13 |
| AAPC-glycolate-FG | 14 |
| (AAPCXaaFG) | |
| KAAPC-glycolate-FG | 15 |
| (KAAPCXaaFG) | |

Modifications to the peptide tags are intended to be encompassed. For example, a “glycolate” as shown in SEQ ID NOs: 14 and 15 above in TABLE 1, represents a —O—C—C(═O)-modification of the peptide bond between the C and F (i.e. C-glycolate-F) residues to provide a subtiligase acylation site (i.e. —C(═O)—O—) that would not otherwise be present in the peptide bonds of the peptide⁸¹. The typical peptide bond (boxed in area) between a cysteine (C) and a phenylalanine (F) would appear as follows:

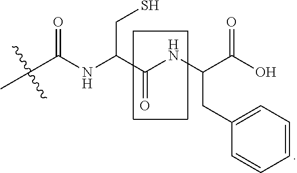

Whereas, the “glycolate” modification appears as the —O—C—C(═O)— (i.e. boxed in area) in the following:

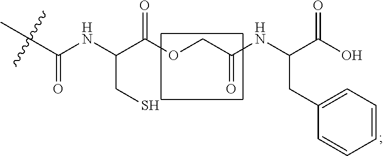 [SMILES: *C(=O)NC(CS)C(=O)OCC(=O)NC(Cc1ccccc1)C(=O)O]

and provides a subtiligase acylation site (i.e. —C(═O)—O—) that does not otherwise occur in between the C and F residues and is shown in the boxed in area of the following:

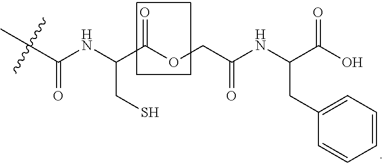 [SMILES: *C(=O)NC(CS)C(=O)OCC(=O)NC(Cc1ccccc1)C(=O)O]

The term “peptide linking group” as used herein, refers to a group that facilitates linking the peptide tag to the polyglycerol or functionalized polyglycerols as described herein. The peptide linking group may be selected from one or more of: an amide group; an alcohol group; an amine group; a thiol group; an azide group; an alkyne group; an alkene group; a carboxylic acid group; an aldehyde group; a ketone group; a halogen group; an isocyanate group; an isothiocyanate group; an oligoethylene glycol spacer; and a Michael acceptor/donor group.

The term “sialic acid” as used herein, refers to a family of derivatives of neuraminic acid, an acidic sugar with a 9-carbon backbone and are typically found attached via an alpha-linkage, to the terminal ends of glycoconjugates on the cell surface or on secreted soluble proteins. Sialic acids have important roles in cellular communication and can mediate or modulate a wide variety of physiological and pathological processes, mostly in animal tissues. Sialic acids include but are not limited to N-acetyl-neuraminic acid (Neu5Ac) and the glycoconjugates (for example, oligosaccharides, glycoproteins and glycolipids or sialic acid derivatives). Furthermore, sialic acids commonly form part of glycoproteins, glycolipids or gangliosides, where they are often terminally attached to the end of sugar chains at the surface of cells or soluble proteins. There are more than 50 types of sialic acids, all of which may be derived from neuraminic acid

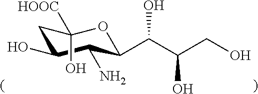

by substituting its amino group or one of its hydroxyl groups. In general, the amino group bears either an acetyl or a glycolyl group, but other modifications have been described. The hydroxyl substituents may include acetyl, lactyl, methyl, sulfate, and phosphate groups. The most common substituents to the nine carbon backbone are as follows: C-1 (i.e. COO) may have an H, may form lactones with hydroxyl groups on the same molecule or on other glycans, may form lactams with a free amino group at C-5, or may be a tauryl group; C-2 (i.e. CO) may have an H, an alpha linkage to Gal(3/4/6), GalNAc(6), GlcNAc(4/6), Sia (8/9), or 5-O-Neu5Gc, an oxygen linked to C-7 in 2,7-anhydro molecule, or an anomeric hydroxyl eliminated in Neu2en5Ac (double bond to C-3); C-4 (i.e. CO) may have an H, an -acetyl group, anhydro to C-8, Fuc; or Gal group; C-5 (i.e. C) may have an amino group, N-acetyl group (i.e. like Neu5Ac), N-glycolyl; hydroxyl group, N-acetimidoyl group, N-glycolyl-O-acetyl group, N-glycolyl-O-methyl group, or a N-glycolyl-O-2-Neu5Gc group; C-7 (i.e. CO) may have an H, an -acetyl group, an anhydro to C-2, substituted by amino or a N-acetyl in Leg; C-8 (i.e. CO) may have an H, an acetyl f=group, an anhydro to C-4; -methyl group, a -sulfate group, a Sia group or a Glc f=group; and C-9 (i.e. CO) may have an H, an -acetyl group, a -lactyl group, a -phosphate group, a -sulfate group, a Sia group or an OH substituted by H in Leg.

Common sialic acids are N-acetylneuraminic acid (Neu5Ac) having the structure

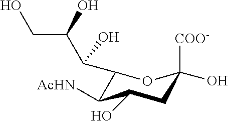

or 2-ket-3-deoxynonic acid (Kdn) having the structure

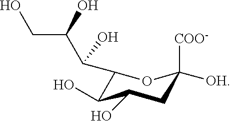

The term “immunosuppression” or “immune modulation” is used herein as it is normally understood to a person of ordinary skill in the art and refers to the suppression or modulation of the immune system of an organ, a tissue or a cell recipient. Furthermore, as used herein the suppression or modulation of a recipient's immune system may be localized immunosuppression, localized immune modulation and/or localized inflammation prevention.

As used herein, “perfusion” or “perfusing” refers to permeating an organ, usually a transplant organ, with a fluid by circulating the fluid through blood vessels of the organ. An important goal in organ preservation is to increase the number of available transplantable organs. Typically, organs are kept in cold storage, but this has potential diffusional limitations, and thus cold perfusion systems have been developed. Furthermore, near-normothermic systems are also being used to enhance the functional preservation of solid organs including livers, lungs, hearts and kidneys.

The term “preservation solution” is used herein as it is normally understood to a person of ordinary skill in the art and often refers to any solution the that can be used to preserve transplant organs, tissues or cells and may be useful in minimizing the damaging effects of cold ischemia and/or warm reperfusion on organs or tissue or cells during the transplant process. As used herein the term “preservation solution” is meant as a general catch-all term and to encompass, but not be limited to: perfusion fluid; organ transplant perfusion fluid; transplant solution; preservation solution; organ preservation solution; transplant preservation solution; and preservations solution for transplants.

A “perfusion fluid” as used herein is a subset of “preservation solution” and is used for in transplantation of an organ or a tissue and may include a buffered extracellular solution. The buffered extracellular solution may be selected from: Steen™; Perfadex™; Perfadex Plus™; EuroCollins solution; Histidine-Tryptophan-Ketoglutarate (HTK) solution; University of Wisconsin solution (UW); Celsior™ solution; Kidney Perfusion solution (KPS-1); Kyoto University solution; IGL-1 solution; and Citrate solution.

The preservation solutions as described herein may have a pH between about 2.0 and about 9.0 or between about 6.5 and about 7.5. The transplant preservation solutions as described herein may be in aqueous solution, wherein the polylgycerol comprises about 0.01% by weight to about 50% by weight of the solution or between about 1.25% by weight to about 20% by weight of the solution.

The preservation solution described herein may include a functionalized linear polyglycerol polymer compound as described herein. The functionalized linear polyglycerol polymer compounds may be flexible, hydrophilic aliphatic polyether polymer, that may be synthesized in linear, form with precise control of molecular weight. The functionalized linear polyglycerol polymer compounds and derivatives thereof may have an excellent biocompatibility profile and may also alternatively have multi-functionality.

The preservation solution as described herein may further comprise one or more electrolytes, one or more amino acids, one or more diffusion agents, and/or one or more osmotic agents. The diffusion agent or osmotic agent may comprise sodium, chloride, lactate, bicarbonate, a bicarbonate producing agent, calcium, potassium, magnesium, dextrose, fructose, glycerol, sorbitol, manitol, L-carnitine, bovine serum albumin (BSA), maltose, maltotriose, maltopentose or xylitol.

The preservation solution as described herein may be used in the process of organ transplantation. The organ transplantation may be conducted for a mammal.

The preservation solution as described herein may be included in a kit for formulating a preservation solution. The kit may comprise a lyophilized polyglycerol functionalized with one or more sialic acids, a linker, and a peptide tag in combination with a cell surface ligating enzyme as described herein and instructions for using the lyophilized polyglycerol for formulating the preservation solution. The kit may comprise other components of the preservation solution, including electrolytes, amino acids, one or more other diffusion agents and/or one or more other osmotic agents.

The preservation solution as described herein may be included in a composition. The composition may comprise linear polyglycerol functionalized with one or more sialic acids a linker, and a peptide tag in combination with a cell surface ligating enzyme as described herein and at least one physiologically acceptable salt, buffer, diluent or excipient, for use as a preservation solution. The composition may be in aqueous solution or a lyophilized product.

The solid organ or organ part may be selected from one of the following: lung; kidney; liver; heart; pancreas; intestine; and blood vessel. The solid organ may be a kidney or a lung.

As used herein, the term “cell-surface ligating enzyme” is meant to refer to any enzyme capable of ligating a peptide (for example, a peptide tag as described herein or as could readily be designed to work with the chosen enzyme or enzymes) to the endothelial cell surface of a donor organ as described herein. A number of enzymes are known to a person of skill in the art, for example, but not limited to: sortases; asparagine endopeptidase or asparaginyl endoproteases (for example, OaAEP1); trypsin related enzymes; and subtilisin-derived variants (i.e. subtiligase) 79. Alternatively, transglutaminases as described herein may be used or enzymes described herein or known in the art may be readily engineered to ligate specific peptide sequences to a cell surface. Alternatively, a butelase, a type of asparagine endopeptidase, might be used as a ligating enzyme for cell surface modification, provided that there is a naturally occurring butelase substrate. An enzymatic reaction for ligating the peptide tag to a cell surface is preferably a broad acting enzyme to increase the area of the cell being engineered. However, a person of skill in the art would be able to engineer an enzyme having an optimized substrate specificity and efficiency, for a particular peptide tag and/or for a particular cell surface and/or for a given specific use⁸².

Exemplary transglutaminases may be found at EC 2.3.2.13. For example, a guinea pig liver transglutaminase (gtTGase, Sigma™ T5398) was used in some of the examples (PKPQQFM, GQLKHLEQQEG, and PNPQLPF)^{37,83,84} described herein (accession number: NP_001166573.1; P08587.4). The gtTGase consists of a single polypeptide chain of 691 amino acid residues. It has six potential glycosylation sites (Asn-X-Ser or Asn-X-Thr), but it is not glycosylated. The molecular mass is approximately 76.6 kDa. It is calcium dependent and has several calcium binding sites. The enzyme is inhibited by iodoacetamide and N-ethylmaleimide in the presence of calcium. It catalyzes the incorporation of small molecular weight amines into γ-glutamine sites of proteins. In the absence of small molecular weight amines, it catalyzes the cross linking of proteins that results in the formation of γ-glutamyl-ε-lysine side chain peptides. Liver transglutaminase is a non-zymogenic enzyme. An alternative TGase enzyme was tested microbial transglutaminase (mTGase), which was commercially available (Zedira™ Tool). The mTGase was tested on peptide tag sequences NQEQVSPLTLLK, TVQQEL, QVPL, and QQPL.

TABLE 2
Exemplary Enzyme Sequences

| | SEQ ID | |
|---|---|---|
| Enzyme | NO | Sequence |
| Guinea pig liver | 16 | MAEDLILERCDLQLEVNGRDHRTADLCRERLVLRRGQPFWLTLHFE |
| transglutaminase | | GRGYEAGVDTLTFNAVTGPDPSEEAGTMARFSLSSAVEGGTWSASA |
| Cavia porcellus | | VDQQDSTVSLLLSTPADAPIGLYRLSLEASTGYQGSSFVLGHFILL |
| (Accession: | | YNPRCPADAVYMDSDQERQEYVLTQQGFIYQGSAKFINGIPWNFGQ |
| NP_001166573.1) | | FEDGILDICLMLLDTNPKFLKNAGQDCSRRSRPVYVGRVVSAMVNC |
| | | NDDQGVLQGRWDNNYSDGVSPMSWIGSVDILRRWKDYGCQRVKYGQ |
| | | CWVFAAVACTVLRCLGIPTRVVTNFNSAHDQNSNLLIEYFRNESGE |
| | | IEGNKSEMIWNFHCWVESWMTRPDLEPGYEGWQALDPTPQEKSEGT |
| | | YCCGPVPVRAIKEGHLNVKYDAPFVFAEVNADVVNWIRQKDGSLRK |
| | | SINHLVVGLKISTKSVGRDEREDITHTYKYPEGSEEEREAFVRANH |
| | | LNKLATKEEAQEETGVAMRIRVGQNMTMGSDFDIFAYITNGTAESH |
| | | ECQLLLCARIVSYNGVLGPVCSTNDLLNLTLDPFSENSIPLHILYE |
| | | KYGDYLTESNLIKVRGLLIEPAANSYVLAERDIYLENPEIKIRVLG |
| | | EPKQNRKLIAEVSLKNPLPVPLLGCIFTVEGAGLTKDQKSVEVPDP |
| | | VEAGEQAKVRVDLLPTEVGLHKLVVNFECDKLKAVKGYRNVIIGPA |
| Guinea pig liver | 17 | MAEDLILERCDLQLEVNGRDHRTADLCRERLVLRRGQPFWLTLHFE |
| transglutaminase | | GRGYEAGVDTLTFNAVTGPDPSEEAGTMARFSLSSAVEGGTWSASA |
| Cavia cutleri | | VDQQDSTVSLLLSTPADAPIGLYRLSLEASTGYQGSSFVLGHFILL |
| (Accession: | | YNPRCPADAVYMDSDQERQEYVLTQQGFIYQGSAKFINGIPWNFGQ |
| P08587) | | FEDGILDICLMLLDTNPKFLKNAGQDCSRRSRPVYVGRVVSAMVNC |
| | | NDDQGVLQGRWDNNYSDGVSPMSWIGSVDILRRWKDYGCQRVKYGQ |
| | | CWVFAAVACTVLRCLGIPTRVVTNFNSAHDQNSNLLIEYFRNESGE |
| | | IEGNKSEMIWNFHCWVESWMTRPDLEPGYEGWQALDPTPQEKSEGT |
| | | YCCGPVPVRAIKEGHLNVKYDAPFVFAEVNADVVNWIRQKDGSLRK |
| | | SINHLVVGLKISTKSVGRDEREDITHTYKYPEGSEEEREAFVRANH |
| | | LNKLATKEEAQEETGVAMRIRVGQNMTMGSDFDIFAYITNGTAESH |
| | | ECQLLLCARIVSYNGVLGPVCSTNDLLNLTLDPFSENSIPLHILYE |
| | | KYGDYLTESNLIKVRGLLIEPAANSYVLAERDIYLENPEIKIRVLG |
| | | EPKQNRKLIAEVSLKNPLPVPLLGCIFTVEGAGLTKDQKSVEVPDP |
| | | VEAGEQAKVRVDLLPTEVGLHKLVVNFECDKLKAVKGYRNVIIGPA |
| transglutaminase | 18 | MHRRIHAVGQARPPPTMARGKETKSYAETYRLTADDVANINALNES |
| Streptomyces | | APAASSAGPSFRAPDSDDRVTPPAEPLDRMPDPYRPSYGRAETVVN |
| mobaraensis | | NYIRKWQQVYSHRDGRKQQMTEEQREWLSYGCVGVTWVNSGQYPTN |
| (accession | | RLAFASFDEDRFKNELKNGRPRSGETRAEFEGRVAKESFDEEKGFQ |
| Q6E0Y3) | | RAREVASVMNRALENAHDESAYLDNLKKELANGNDALRNEDARSPF |
| | | YSALRNTPSFKERNGGNHDPSRMKAVIYSKHFWSGQDRSSSADKRK |
| | | YGDPDAFRSAPGTGLVDMSRDRNIPRSPTSPGEGFVNFDYGWFGAQ |
| | | TEADADKTVWTHGNHYHAPNGSLGCHACLTRASSATGSEGYSDFDR |
| | | GEPYVVSPSPSPRMLEHRPRQGKAGLA |
| transglutaminase | 19 | MHRRIHAVGQARPPPTMARGKETKSYAETYRLTADDVANINALNES |
| Streptomyces | | APAASSAGPSFRAPDSDDRVTPPAEPLDRMPDPYRPSYGRAETVVN |
| mobaraensis | | NYIRKWQQVYSHRDGRKQQMTEEQREWLSYGCVGVTWVNSGQYPTN |
| (accession | | RLAFASFDEDRFKNELKNGRPRSGETRAEFEGRVAKESFDEEKGFQ |
| AAT65817) | | RAREVASVMNRALENAHDESAYLDNLKKELANGNDALRNEDARSPF |
| | | YSALRNTPSFKERNGGNHDPSRMKAVIYSKHFWSGQDRSSSADKRK |
| | | YGDPDAFRSAPGTGLVDMSRDRNIPRSPTSPGEGFVNFDYGWFGAQ |
| | | TEADADKTVWTHGNHYHAPNGSLGCHACLTRASSATGSEGYSDFDR |
| | | GEPYVVSPSPSPRMLEHRPRQGKAGLA |
| transglutaminase | 20 | MSQRGRTLVFAALGAVMCTTALMPSAGAATGSGSGSGTGEEKRSYA |
| Streptomyces | | ETHRLTADDVDDINALNESAPAASSAGPSFRAPDSDERVTPPAEPL |
| mobaraensis | | DRMPDPYRPSYGRAETIVNNYIRKWQQVYSHRDGRKQQMTEEQREW |
| (accession | | LSYGCVGVTWVNSGQYPTNRLAFAFFDEDKYKNELKNGRPRSGETR |
| AA048277) | | AEFEGRVAKDSFDEAKGFQRARDVASVMNKALENAHDEGAYLDNLK |
| | | KELANGNDALRNEDARSPFYSALRNTPSFKDRNGGNHDPSKMKAVI |
| | | YSKHFWSGQDRSGSSDKRKYGDPEAFRPDRGTGLVDMSRDRNIPRS |
| | | PTSPGESFVNFDYGWFGAQTEADADKTVWTHGNHYHAPNGSLGAMH |
| | | VYESKFRNWSDGYSDFDRGAYVVTFVPKSWNTAPDKVTQGWP |

An “immobilized enzyme” as used herein is an enzyme attached to surface, which may be an inert, insoluble material. Immobilization of enzymes can provide increased resistance to changes in conditions such as pH, temperature etc. and assist in their removal following use and for enzyme re-use.

Immobilization of an enzyme may be accomplished by various ways (for example, affinity-tag binding, surface adsorption on glass, resin, alginate beads or matrix, bead, fiber or microsphere entrapment, cross-linking to a surface or other enzymes and covalent binding to a surface).

Polyglycerol is a clear, viscous liquid. At room temperature, it is highly viscous and essentially non-volatile. Linear polyglycerols are of a compact nature in solution and highly soluble in water.

The polyglycerols as described herein may be functionalized. Functionalized linear polyglycerols may include polymers which contain sialic acid, sulfated residues, sulfates, peptides or sugars, which have been added to the polymer. Such regions may be provided by derivatizing the hydroxyl groups of the polymer. A functional derivative may be bound to about 0.01% to about 100% of hydroxyl groups on the linear polyglycerol, or to about 1% to about 40% of hydroxyl groups on the linear polyglycerol. By adding such groups to the linear polyglycerol, the number of hydroxyl groups may no longer be equal to the number of repeat units in the linear polyglycerol.

The term “hyperbranched polyglycerol” (HPG) is used herein as it is normally understood to a person of ordinary skill in the art and often refers to a polyglycerol having a degree of branching between about 0.5 and about 0.7. HPGs are water-soluble branched polyether polymers that have been used for many medical applications, such as restoring the circulation volume as an albumin substitute and in peritoneal dialysis solution as a primary osmotic agent. HPG is a highly water soluble (>400 mg/mL) and a compact polymer, has an equal or better biocompatibility profile compared to polyethylene glycol (PEG), HPG has low intrinsic viscosity that is similar to that of proteins and is approximately 10-times lower than that of linear polymers (i.e. PEG, HES, dextran). HPGs are herein contemplated as an alternative to linear polyglycerols for functionalization (i.e. with sugars, sialic acids, sulfonated, otherwise negatively charged, with peptide tags as described herein) and used for CSE of organs, tissues and cells.

The rejection of transplant organs results from a complex series of actions by the recipient's innate and adaptive immune systems. T cells are key to both processes, whereby when recipient's T cells recognize donor antigens (i.e. allorecognition) this can initiate organ rejection (i.e. allograft rejection), because, once the recipient's T cells become activated, they undergo clonal expansion, differentiate into effector cells, and migrate into the allograft, which then promotes tissue destruction. Furthermore, CD4 T cells help B cells produce alloantibodies. Furthermore, B cells and anti-HLA antibodies have recently been shown to play an important role in both acute and chronic allograft rejection. However, regulatory T cells (Tregs) mediate the recipient's immune response via a number of mechanisms (i.e. production of suppressor cytokines, direct suppression of effector cells, modulating antigen presenting cells (APCs) bystander suppression, and by regulating inflammation). The immune system may be diverted from causing immune mediated damage to a transplant organ via a number of mechanisms (for example, immune cloaking). Immune cloaking as used herein refers to cell surface engineering (CSE) techniques that assist a transplant organ to evade immune detection. Generally, this evasion may occur by: preventing T cells from recognizing donor antigens; preventing the binding of effector cells and their products (for example, antibodies) to the allograft, which can reduce inflammation and damage to the allograft; inducing Tregs to suppress the immune response to the allograft; or a combination of thereof. Furthermore, where initial immune cloaking occurs, immune conditioning may result whereby the immune system no longer recognizes the donor antigens, even after the CSE may no longer be present or remain completely intact.

Any terms not directly defined herein shall be understood to have the meanings commonly associated with them as understood within the art of the invention.

Various alternative embodiments and examples are described herein. These embodiments and examples are illustrative and should not be construed as limiting the scope of the invention.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way.

Further definitions and meanings of abbreviations
CIS cold ischemia storage
CSE cell-surface engineering
CAR-T cell chimeric antigen receptor T cell
CuAAc Cu(I) catalyzed azide alkyne cycloaddition
DAMPs danger associated molecular patterns
DGF delayed graft function
gtTGase guinea pig liver tissue transglutaminase
H&E hematoxylin and eosin
HLA human leukocyte antigen
IRI ischemia-reperfusion injury
LDH lactate dehydrogenase
LPG linear polyglycerol
NK cells natural killer cells
PBMCs human peripheral blood mononuclear cells
RNA-seq RNA sequencing
ROS reactive oxygen species
SCS static cold storage
Siglec sialic acid-binding, immunoglobulin (Ig)-like lectin
TLR toll-like receptor
UT Untreated
UW solution organ preservation solution
WGA wheat germ agglutinin

Various alternative embodiments and examples are described herein. These embodiments and examples are illustrative and should not be construed as limiting the scope of the invention.

### Materials and Methods

### Materials

All chemicals were purchased from Sigma-Aldrich™ (ON, Canada) unless otherwise mentioned. University of Wisconsin (UW) solution was purchased from Organ Recovery Systems™ (Itasca, IL, USA). Blood was collected from healthy and consenting donors at the Centre for Blood Research with protocol approval from the University of British Columbia clinical ethics committee in vials containing EDTA or sodium citrate. All peptides were purchased fully characterized (RP-HPLC, ESI-MS) by Canpeptide Inc.™ (Montreal, QC, Canada) and used without further purification. Guinea pig tissue transglutaminase and Histopaque™-1077 were purchased from Sigma-Aldrich™ Canada. Recombinant human TNF alpha protein was purchased from Abcam™ and recombinant human IL-2 was purchased from Cedarlane™. Anti-SHP-1 mAb, Human IL-10 DuoSet ELISA Kit, Human IL-6 Quantikine ELISA Kit™ and Proteome Profiler™, Panel A were obtained from R&D Systems™. EasySep™ isolation kit for CD56+ cells was obtained from STEMCELL Technologies™. Antibodies were obtained as follows; PE-Cy™5 mouse anti-human CD54, FITC-conjugated anti-human CD45, APC-conjugated anti-CD8a mAb and FITC-conjugated anti-CD69 mAb are from BD Biosciences™. FITC-conjugated anti-human CD8a is from STEMCELL Technologies™; CellTracker™ Green (CTG) CMFDA Dye is from Invitrogen™; APC-conjugated anti-human siglec-7 is from Biolegend™; and CellMask™ deep red plasma membrane stain, Anti-Human CD8+ Dynabeads™ and rabbit anti-goat Dylight 800™ secondary antibody are from Thermo Scientific™.

### Cell Culture Materials

All cell culture-related media and supplements (Trypsin-EDTA, Dulbecco's phosphate-buffered saline (DPBS), HI fetal bovine serum (FBS), penicillin/streptomycin (P/S), and Dulbecco's modified eagle medium (DMEM)) were received from Life Technologies Inc. unless otherwise specified. Ea.hy926 cells were purchased from American Type Culture Collection™ (ATCC CRL-2922 Manassas, VA) and used up to a passage number of 50. The cells were cultured using DMEM cell media containing 10% FBS and 1% P/S in tissue culture treated T-75 flasks at 37° C. and 5% CO2. Upon reaching 70% confluence, cells were dissociated with 0.25% trypsin and 0.05% EDTA (Gibco™, 25300062), pelleted by centrifugation at 300 g and resuspended with complete DMEM medium. HMEC-1 cells were obtained from ATCC and used up to a passage number of 20. The cells were cultured using MCDB 131cell media containing 10 ng/mL epidermal growth factor, 1 μg/mL hydrocortisone, 10 mM glutamine, 10% FBS and 1% P/S in tissue culture treated T-75 flasks at 37° C. and 5% CO₂. HUVEC cells were obtained from ATCC and used up to a passage number of 10. The cells were cultured using EBM-2 basal media (CC-3156, Lonza™) completed with EGM-2 SingleQuots Supplements™ (CC-4176, Lonza™) in tissue culture treated T-75 flasks at 37° C. and 5% CO₂. To obtain T cells, EDTA anticoagulated blood from healthy donors was subjected to ficoll density gradient centrifugation Histopaque™-1077 (Sigma Aldrich™). PBMCs were cultured for 24 h at 37° C. and 5% CO₂ in RPMI-1640 containing 10% FBS (v/v), 1000 U/mL recombinant human IL-2 (Cedarlane™) and 1% P/S. Prior to fluorescent labelling, cultured PBMCs were tested for absence of platelets by labelling an aliquot of cells with FITC-labelled anti-human CD45 (1:200 Beckman Coulter™) and analyzed by flow cytometry (CytoFLEX™, Beckman Coulter™). T-cells were isolated from PBMCs through negative selection using immunomagnetic beads (STEMCELL™, 19661). To obtain platelets, Whole blood samples were collected in blood vacutainers containing sodium citrate. Blood vials were centrifuged at 200 g for 15 minutes and the serum layer and buffy coat were collected. The remaining suspension was further centrifuged at 2000 g for 20 minutes to collect plasma. The isolated suspension was diluted using 1 volume equivalent of CGS buffer (120 mM sodium chloride, 10 mM trisodium citrate, 30 mM dextrose, pH 6.5) and further centrifuged at 500 g for 15 minutes to pellet platelets. An aliquot of the platelet solution was labelled using FITC-labelled anti-human CD42a (1:20 dilution) and analyzed by flow cytometry. Platelet samples of 99% purity or higher were implemented for further study. Human pancreatic islets were generously donated by the Kieffer lab (University of British Columbia).

### General Procedure for Cell Surface Engineering

Cells were washed twice with cold DPBS and incubated with media freshly supplemented with 3 mM GSH, 5 mM CaCl₂, 0.2 U/mL gtTGase and 0.5 mM PEG-Q or LPGS-Q, unless otherwise noted; for platelets, a mixture of DPBS and CGS buffer (3:1 v/v). The solutions were mixed thoroughly and incubated at 4° C. for 30 minutes under static conditions. The cell supernatant was collected and the cells were washed three times with PBS and subjected to further analysis. The assessment of polymer attachment to endothelial cells are assessed by ammonia assay.

### Techniques

All reactions with air and/or water sensitive reagents were performed in a Schlenk flask under dry argon atmosphere. Absolute molecular weights of the polymers were determined by Gel Permeation Chromatography (GPC) on a Waters e2695™ separation module fitted with a DAWN HELEOS-II™ multiangle laser light scattering (MALLS) detector coupled with Optilab T-rEX™ refractive index detector, both from Wyatt Technology™. GPC analysis was performed using Waters™ ultrahydrogel 7.8×300 columns (guard 250 and 120) and 0.1 N NaNO₃ at pH 8.5 (10 mM phosphate buffer) as the mobile phase. The dn/dc (0.12) for polyglycerols was used from previously measured literature values.^{1 1}H nuclear magnetic resonance (NMR) spectra were recorded on a Bruker Avance 300™ MHz NMR spectrometer using deuterated solvents (Cambridge Isotope Laboratories™, 99.8% D). NMR spectra were obtained by scanning 128 times at atmospheric conditions. Chemical shifts were referenced to the residual solvent peak. The ACD/NMR processor spectroscopic software was used for data handling. Fourier transform infrared spectroscopy (FTIR) spectra were recorded on a Bruker TENSOR II FTIR spectrometer with a resolution of 4 cm-1. Polymer samples were incorporated into a KBr pellet prior to analysis. FTIR spectra were obtained by scanning 64 times at atmospheric conditions. The OPUS™ spectroscopic software was used for data handling. Absorbance and fluorescence readings were acquired using a SpectraMAX™ multi-mode M3 plate reader from Molecular Devices LLC™. Flow cytometry profiles were acquired using a 3-laser CytoFLEX™ flow cytometer from Beckman Coulter Life Sciences™ (10,000 events).

### Chemical Synthesis of Linear Polyglycerol and its Modification:

### Preparation of Ethoxy Ethyl Glycidyl Ether Monomer (EEGE)

In a round bottom flask, dry glycidol (40 mL, 0.63 mol) was dissolved in ethyl vinyl ether (250 mL, 2.19 mol) and the solution was cooled in an ice bath to 0° C. Under constant stirring, p-toluenesulfonic acid (1.15 g, 5.8 mmol) was added portion wise, maintaining the temperature below 20° C. The reaction was allowed to reach room temperature after the addition of p-toluenesulfonic acid was done and stirred for 4 h. The reaction was then quenched by addition of 100 mL saturated NaHCO₃ solution. The organic phase was then separated and dried over Na₂SO₄. Fractionated vacuum distillation yielded the product as a colorless liquid (percent yield=90%).

¹H-NMR (300 MHz, CDCl₃): δ=4.9 (m, 1H), 3.76-3.32 (m, 4H), 3.07 (m, 1H), 2.73 (m, 1H,), 2.53 (m, 1H J=5.03, 2.74 Hz), 1.25 (t, 3H, J=6.0 Hz), 1.1 ppm (t, 3H, J=6.0 Hz)

### Preparation of Ethoxyethyl Glycidyl Ether

To a solution of glycidol, add 3.5 equivalents of ethyl vinyl ether and cool to 0° C. While ensuring the temperature stays below 20° C., p-toluenesulfonic acid was added slowly. The mixture was left to react for 4 hours while allowing it to return to room temperature. The reaction was then quenched using saturated sodium bicarbonate. The organic phase of the reaction mixture was extracted and the solution was concentrated in vacuo. The concentrated solution was distilled at 40° C., 0.6 Torr. The clear liquid was characterized using ¹H NMR.

¹H NMR (300 MHz, CDCl₃): δ=1.03 (t, 3H), 1.15 (t, 3H), 2.44 (m, 1H), 2.62 (m, 1H) 2.96 (m, 1H), 3.22-3.41 (m, 2H), 3.47-3.68 (m, 2H), 4.59 (m, 1H)

### Preparation of α-Azido Linear Polyglycerol (N₃-LPG)

The preparation of linear polyglycerol was carried out according to the previously published method by Gervais, M. et al.⁸⁵ Tetrabutylammonium azide (0.11 g, 0.39 mmol) was added to a flame dried Schlenk flask and dried under reduced pressure for three hours at 90° C. Toluene (5 mL) and EEGE monomer (16.5 mL, 0.11 mol) were added via syringe under argon. The reaction mixture was cooled down in an ice/salt bath to −10° C. and activated by the fast addition of triisobutylaluminum (TIBA, 1 M in hexane, 1.60 mL, 1.59 mmol). After addition of TIBA, the mixture was stirred overnight, returning to room temperature. Upon reaction completion, 1 mL of methanol was used to quench the reaction and the solution was stirred for another 30 minutes. Removal of aluminum as the insoluble Al(OH)₃ salt was carried out by cooling the solution to 0° C. followed by the addition sodium hydroxide solution (15 wt. %). The resulting solution was dried using MgSO₄, followed by filtration and removal of the solvent in vacuo. The viscous, light yellow residue was characterized by ¹H-NMR and carried forward without further purification.

¹H-NMR (300 MHz, CDCl₃): δ=4.70 (m, 1H), 3.70-3.41 (m, 7H), 1.29-1.14 (m, 6H) In order to remove the protecting group, the polymer was dissolved in a solution of 3.7% HCl in ethanol (100 mg/mL) and left overnight to deprotect. The light yellow solution was neutralized with saturated NaHCO₃, dialyzed immediately against 1K MWCO tubing and lyophilized to generate α-azido linear polyglycerol (LPG-N₃).

¹H-NMR (300 MHz, D₂O): δ=3.72-3.59 ppm (m, LPG backbone, 5H).

Mₙ=14, 900 Da, M_{w}/Mₙ=1.23.

Preparation of propargylated polyglycerols (N₃-LPG-Alkyne)

LPG-N₃ (14,900 Da, 830 mg, 0.06 mmol) was added to a 50 mL flame dried Schlenk flask, dried overnight at 50° C. and purged in argon. The polymer was subsequently dissolved in 33 mL anhydrous dimethylformamide (DMF). After complete dissolution of the polymer, NaH (96 mg, 3.9 mmol) was added in three separate batches under vigorous stirring to afford a cloudy solution. The temperature was elevated to 65° C. and allowed to react for 3 hours under argon. Propargyl bromide (330 μL, 3.0 mmol) was added dropwise to the solution and the mixture was allowed to react for 24 hours under argon. Methanol was added to quench the reaction and the mixture was precipitated 2 times in cold ether. The precipitate was collected via centrifugation. The pellet was then re-dissolved and dialyzed against 1K MWCO tubing in MeOH. Following dialysis, the residual MeOH was removed in vacuo and subjected to structural analysis. ¹H-NMR (300 MHz, MeOD): δ=4.17 pp (m, CH₂C≡CH, 2H) δ=3.66-3.51 ppm (m, LPG backbone, 5H). ˜35 alkynes per LPG (71% conversion)

Preparation of α-amino linear polyglycerol (LPG-NH₂)

Tetrabutylammonium azide (0.39 mmol) was added to a flame dried Schlenk flask and dried in vacuo at 90° C. for 3 hours. Toluene (anhydrous) and ethoxyethyl glycidyl ether (110 mol) was introduced via syringe under argon. The reaction was cooled to −10° C. and triisobutylaluminum (1M in hexane, 1.59 mmol) was added rapidly. The reaction was then left to stir overnight and allowed to return to room temperature. The reaction was quenched using 1 mL of methanol and allowed to stir for 30 minutes. Aluminum was removed by forming aluminum hydroxide through the cooling of the solution to 0° C. and adding of sodium hydroxide (15 wt. %). The final solution was dried using magnesium sulfate and filtered, and the solvent was removed in vacuo. The viscous, light yellow product was characterized using ¹H NMR.

¹H-NMR (300 MHz, CDCl₃): δ=1.07 (t, 3H), 1.18 (t, 3H), 3.32-3.59 (m, 7H), 4.59 (q, 1H) Deprotection of poly(ethoxyethyl glycidyl ether) was carried out by dissolving the previous product in a solution of 10% HCl in ethanol (50 mg/mL) and left stirring for 4 hours. Solvent was removed in vacuo and the viscous solution was subjected to dialysis against 3.5 K molecular weight cut-off (MWCO) regenerated cellulose tubing and lyophilized. The removal of the protecting group was detected using ¹H NMR.

To convert the azide group to an amine group, the polymer was dissolved in pyridine (50 mg/mL). Triphenylphosphine (1.5 mol eq.) was added and the reaction was left to stir for 48 hours at room temperature. After the reaction was done, the solvent was removed in vacuo and the product was extracted against chloroform. The solution was then dialyzed against 3.5 k MWCO regenerated cellulose tubing in methanol and lyophilized. The removal of triphenylphosphine was detected using ³¹P NMR and the molecular weight was determined using GPC.

¹H NMR (300 MHz, CD₃OD): δ=3.53-3.72 (polyglycerol)

Preparation of α-amino propargylated linear polyglycerol (NH₂-LPG-Alkyne)

N₃-LPG-Alkyne (650 mg, 0.045 mmol) was dissolved in 15 mL pyridine and PPh₃ (140 mg, 0.52 mmol) was added to the solution. The reaction was stirred vigorously under ambient conditions for 48 hours. Upon reaction completion, one volume equivalent of methanol was added to the flask and the solution was dialyzed against 1 K MWCO tubing in methanol and dried in vacuo to afford the product. Conversion of azide to amine was confirmed using fourier transform infrared spectroscopy (FTIR).

Preparation of linear polyglycerol glutamine (LPG-Q)

LPG-NH₂ was added to a flame dried Schlenk flask and dried overnight under pressure at 50° C. The polymer was then dissolved in dimethyl formamide (DMF, anhydrous) under argon at room temperature. In a second flame dried Schlenk flask, peptide GLQ(trt)Q(trt)Q(trt)G-COOH (1.5 mol eq.), 1-hydroxybenzotriazole (HOBt, 1.5 mol eq.), Benzotriazol-1-yloxy)tris(dimethylamino)phosphonium hexafluorophosphate (BOP, 1.5 mol eq.), and N-ethyldiisopropylamine (DIPEA, 4.5 mol eq.) were introduced via syringe under argon, dissolved in DMF (anhydrous), and left to stir for 30 minutes at room temperature. The contents of the second flask were transferred into the flask containing the polymer by cannula over 5 minutes. The reaction was left to react for 48 hours under argon at room temperature. The reaction was quenched by adding acetic anhydride (1.25 mol eq.) and left to stir for 60 minutes. The reaction mixture was purified by precipitation using a cold ether: acetone (50:50 v/v) solution three times and the precipitate was collected via centrifugation.

Deprotection of trityl protected glutamines was done by dissolving the polymer in deprotection solution (TFA:TIS:H₂O 92:6:2 v/v) (50 mg/mL) and left to stir for 3 hours at room temperature. TFA was removed in vacuo and the sample was centrifuged to remove any precipitates, filtered, dialyzed against 3.5 K MWCO regenerated cellulose tubing and lyophilized. The removal of the trityl groups was confirmed using ¹H NMR.

Preparation of Linear polyglycerol sulfate glutamine (LPGS-Q)

LPG-NH₂ was added to a flame dried Schlenk flask overnight under pressure at 50° C. The polymer was then dissolved in DMF (anhydrous) under argon at room temperature. In a second flame dried Schlenk flask, peptide GLQ(trt)Q(trt)Q(trt)G-COOH (1.5 mol eq.), HOBt (1.5 mol eq.), BOP (1.5 mol eq.), and DIPEA (4.5 mol eq.) were dissolved in DMF (anhydrous), introduced via syringe under argon, and left to stir for 30 minutes at room temperature. The contents of the second flask were transferred into the flask containing the polymer by cannula over 5 minutes. The reaction was left to react for 48 hours under argon at room temperature. Upon completion, the reaction temperature was elevated to 65° C. Sulfur trioxide pyridine complex (2 mol eq. per —OH group) was dissolved in DMF (anhydrous) to yield a 0.6 M solution. This solution was added via syringe over 30 minutes using a syringe pump under argon and left to stir for 48 hours. Upon completion, the reaction was cooled to room temperature, quenched by adding acetic anhydride (1.25 mol eq.) and left to stir for 60 minutes. The product was precipitated by dissolving the final mixture in 0.5 volume equivalents of water and 1 volume equivalent of precipitation solution (ethanol:NaOH:NaCl 343:6.33:1 n/n) three times, and the precipitate was collected via centrifugation. Deprotection of trityl protected glutamines was done by dissolving the polymer in deprotection solution (TFA:TIS:H₂O 92:6:2 v/v) (50 mg/mL) and left to stir for 2 hours at room temperature. TFA was removed in vacuo and the sample was centrifuged to remove any precipitates, filtered, dialyzed against 3.5K MWCO regenerated cellulose tubing and lyophilized. The removal of the trityl groups was confirmed using ¹H NMR. Degree of sulfation was determined using ¹H NMR.

### Attachment of Q-tag to NH₂-LPG-Alkyne (LPG-Alkyne-Q)

NH₂-LPG-Alkyne (865 mg, 0.06 mmol NH₂ groups) was added to a 50 mL flame dried Schlenk flask (Flask A) dried overnight and purged in argon. The polymer was subsequently dissolved in 9 mL anhydrous DMF. Another 4 mL of DMF was added to another flame dried flask (Flask B). Trityl (trt) protected peptide, Ac-GQ(trt)Q(trt)Q(trt)LGGGGG-OH (0.072 mmol), 1-hydroxybenzotriazole (HOBt, 11 mg, 0.072 mmol), benzotriazol-1-yloxy)tris(dimethylamino)phosphonium hexafluorophosphate (BOP 32 mg, 0.072 mmol), and N-ethyldiisopropylamine (DIPEA 60 μL, 0.72 mmol) was added to flask B and allowed to stir for 30 min at room temperature, after which the contents were transferred to Flask A by cannula over 5 minutes with 1 mL DMF wash. The new mixture in Flask A was left at room temperature under argon for 48 hours. Acetic anhydride (17 μL, 0.18 mmol) was added to quench the reaction and the mixture was stirred for 60 min. The final mixture was precipitated 3 times in cold ether:acetone (50:50 v/v) and the precipitate was collected via centrifugation. The trt protected polymer-peptide conjugate was dissolved in a deprotection mixture containing trifluoroacetic acid (TFA) and triisopropylsilane (TIS) in water (92:6:2 TFA:TIS:H₂O v/v) to a final concentration of 50 mg/mL and the solution was left to react for 3 hours at room temperature. Upon reaction completion, the solution was dried in vacuo to remove TFA dissolved in 5 mL methanol and centrifuged to remove any insoluble ppt. The supernatant was dialyzed (2 MWCO) in methanol overnight, followed by water for a further 24 hours filtered and freeze dried. Conjugates were characterized by ¹H NMR.

¹H NMR (300 MHz, MeOD): δ=3.92-3.59 ppm (m, LPG backbone, 5H), δ=0.91-0.89 ppm (m, leucine —CH₃, 6H)

Fluorescent tag modification of LPG-Alkyne-Q

Aldehyde groups were generated on the LPG-Alkyne-Q terminus through the oxidation of 1,2 diol groups on one end of linear polyglycerol polymer scaffold using NaIO₄. LPG-Alkyne-Q (190 mg, 0.010 mmol) was dissolved in MES buffer (0.1 M, pH 6.5) followed by the addition of NaIO₄ (3.7 mg, 0.017 mmol). The solution was stirred overnight at room temperature protected from light. Following periodate oxidation, the reaction was quenched through the addition of glycerol to a final concentration of 20 mM. BODIPY hydrazide (0.012 mmol) dissolved in DMSO was added to the solution followed by the addition of an aniline stock solution (1 M in DMSO) to a final aniline concentration of 10 mM. The mixture was left to react overnight at ambient temperature in the dark. Following reaction completion, the reducing agent NaCNBH₃ (7.2 mg, 0.12 mmol) was added to the solution and left to stir overnight at room temperature. Following reduction, an excess of glycine was added to a final concentration of 10 mM to quench any remaining aldehyde groups and stirred for another hour. The conjugate solution was then purified through either dialysis (3.5 K MWCO) or Zeba™ spin desalting columns (7 K MWCO) until a negative silver nitrate test was obtained. This process afforded approximately 1 BODIPY-FL group per polymer (100% BODIPY-FL) as assessed by measuring the intensity of a known concentration of polymer solution against as BODIPY-FL standard curve.

Sialylation of Q-tagged polyglycerols (LPG-Q-Sia3Lac, LPG-Q)

The synthesis of clickable 2,3 and 2,6 sialyl oligosaccharides (2,3-Sia-LacC6N₃, 2,6-Sia-LacC6N₃) was carried out through enzymatic elongation with sialyltransferases according to previously published methods using 6-azidohexanyl-β-lactoside (LacC6N₃) as an acceptor.³⁻⁵ LPG-Alkyne-Q (115 mg, 0.00705 mmol) was dissolved in 4.6 mL anhydrous, degassed DMF. To the polymer solution, α2,3-Sia-LacC6N₃, α2,6-Sia-LacC6N₃ or LacC6N₃ (1.25 eq per alkyne) was added followed by a 1:1 CuBr: N,N,N′,N″,N″-pentamethyldiethylenetriamine (PMDETA) solution (20 mol % relative to alkyne content). The vial was purged with argon, capped and allowed to react at room temperature for 24 hours. Excess 2-azidoethanol was added after 24 hours to react with any remaining alkynes. To generate LPG-Q from LPG-Q Alkyne, 3 eq of 2-azidoethanol per alkyne was used to convert all alkyne groups to hydroxyl groups. Excess EDTA was then added to quench the reaction and the solution was dialyzed against 2 MWCO tubing for 24 hours in 1 M NaCl followed by 24 hours in water. The resulting solution was then lyophilized and subjected to structural analysis.

¹H NMR LPG-Q-Sia3Lac (300 MHz, D₂O): δ=1.99 ppm (s, —CH₃, sialic acetyl group, 3H) δ=3.73-3.59 ppm (m, LPG backbone, 5H), 6=0.88-0.84 ppm (m, leucine —CH₃, 6H)

¹H NMR LPG-Q-Sia6Lac (300 MHz, D₂O): δ=1.99 ppm (s, —CH₃, sialic acetyl group, 3H) δ=3.74-3.60 ppm (m, LPG backbone, 5H), 6=0.89-0.84 ppm (m, leucine —CH₃, 6H)

¹H NMR LPG-Q-Lac (300 MHz, D₂O): δ=1.54-1.26 ppm (m, —CH₂CH₂CH₂CH₂CH₂CH₂, C6 linker, 8H) δ=3.73-3.62 ppm (m, LPG backbone, 5H), δ=0.89-0.84 ppm (m, leucine —CH₃, 6H)

Carboxy functionalization of LPG (LPG-COOH)

LPG-NH2 was generated by subjecting LPG-N3 to the protocol as previously described. LPG-NH₂ (672 mg, 0.047 mmol,) was dried in vacuo at 90° C. overnight and subsequently dissolved in anhydrous pyridine (19 mL). Ten molar equivalents of succinic anhydride (470 mg, 0.470 mmol) was added to the reaction which was then allowed to react under argon at room temperature overnight. Upon reaction completion, the polymer was precipitated from the reaction medium with cold acetone and isolated via centrifugation (14,000 g, 4° C.). The polymer was then re-dissolved in distilled water and dialyzed against 1K MWCO tubing, lyophilized and subjected to structural analysis.

Preparation of linear polyglycerol succinimidyl succinate (LPG-SS)

LPG-COOH (161 mg, 0.0113 mmol) and N-hydroxysuccinimide (7.97 mg, 0.0678 mmol) were re-dissolved in anhydrous DMF (9 mL) followed by the addition of N,N′-diisopropylcarbodiimide (8.75 mg, 0.0678 mmol). The solution was left to stir overnight at room temperature under argon atmosphere. Upon reaction completion, the solution was precipitated in acetone (30 mL), isolated by centrifugation (14,000 g, 4° C.), and dried for 10 minutes under reduced pressure. Once isolated, LPG-SS was immediately dissolved in a PBS stock solution and used for cell derivatization.

¹H-NMR (300 MHz, D₂O): δ=3.71-3.63 ppm (m, LPG backbone, 5H), 2.48 (s, COCH2CH2CO)

Ammonia generation assay for enzyme reactivity

The extent of gtTGase ligation was assessed using an ammonia assay kit (Sigma™, MAK310) and used according to the manufacturer's protocols. This kit is used for the quantitative determination of ammonia (NH₃), a side product in the gtTGase reaction.⁶ This assay is based on the o-phthalaldehyde method in which the reagent reacts with NH₃ producing a fluorescent product (λₑₓ=360 nm/λₘ=450 nm), proportional to the NH₃ concentration in the sample.^{7,8} Briefly, PBS was supplemented with 5 mM CaCl₂ and 3 mM dithiothreitol (DTT). In a 96 well plate, containing either endothelial cells or glycine ethyl ether (GEE, amine donor), Q-tagged polymer (acyl donor) and guinea pig liver transglutaminase (gtTGase, Sigma™ T5398) were added to the PBS solution to a final concentration of 5 mM, 0.5 mM and 0.2 U/mL respectively (Vf=100 μL). The reaction was thoroughly mixed and rocked gently at 4° C. for 30 minutes. The sample was diluted four-fold and a 10 μL aliquot of the reaction mixture was then transferred to a tube containing 90 μL complete ammonia assay reagent and mixed thoroughly. The solution was left to react for 15 minutes at ambient temperature in the dark. The fluorescence intensity was measured at λₑₓ=360 nm/λₑₘ=450 nm using a spectrophotometer and the quantity of ammonia in solution was calculated according to Eq. 1:

$$[ {NH}_{3} ] generated = \frac{{F}_{sample} - {F}_{blank}}{slope} \qquad ( Eq . 1 )$$

Where the slope represents the results of a NH₄Cl standard curve generated under identical conditions to the samples and F_{blank} is the fluorescent intensity of supernatants from endothelial cells or GEE alone in the presence of enzyme.

General procedure for cell-surface engineering (enzymatic and chemical) of endothelial surface

Ea.hy 926 cells at 60-800% confluence were trypsinized and plated in a 96 well plate at 15,000 cells/well. Cells reached 100% confluence at day 3 and allowed to grow for a further 4 days at 37° C. and 5% CO₂. Cells were washed twice with cold DPBS and incubated with UW solution freshly supplemented with 3 mM GSH, 5 mM CaCl₂, 0.2 U/mL gtTGase and 0.5 mM LPG-Q or 0.56 mM glycopolymers (LPG-Q-Sia3LacLPG-Q-Sia6Lac and LPG-Q-Lac) unless otherwise noted. The solutions were mixed thoroughly and incubated at 4° C. for 30 minutes under static conditions. The cell supernatant was collected and the cells were washed three times with PBS and subjected to further analysis. The assessment of polymer attachment to endothelial cells are assessed by ammonia assay, flow cytometry and polymer mediated cell-surface camouflage of surface proteins. Polymer attachment using succinimidyl-succinate functionalized LPG (SS-LPG) was carried out under identical conditions without gtTGase (UW solution freshly supplemented with 3 mM GSH, 5 mM CaCl₂, and 0.5 mM SS-LPG).

Flow cytometry on polymer modified endothelial cells (lifetime measurements, ICAM-1 labeling)

To quantify the extent of cell surface engineering, polymers were attached to endothelial surfaces according to the general cell surface engineering procedures outlined in previously using polymer in which 10% were labelled with BODIPY FL™ (ratio of non-labeled polymer: BODIPY labeled polymer=9:1). The cell monolayers were trypsinized and subjected to flow cytometry (CytoFLEX™, Beckman Coulter™) in which 10,000 events were acquired and the median fluorescent intensity (MFI) in the 488 channel was recorded.

For lifetime measurements, polymers were attached to endothelial surfaces according to procedures outlined herein using 0.5 mM LPG-Q (10% BODIPY FL™) and exchanged into phenol-free DMEM (5% FBS, pen/strep) and incubated at 37° C. and 5% CO₂. For each time point the supernatant was removed and saved for further analysis and the cell monolayers were trypsinized and subjected to flow cytometry (CytoFLEX™, Beckman Coulter™) in which 10,000 events were acquired and the median fluorescent intensity (MFI) in the 488 channel was recorded. The fluorescent intensity of the BODIPY-FL Dye in the supernatant was measured (λₑₓ/λₑₘ=503/520 nm using) a plate reader (Spectramax i3™, Molecular Devices™). Intensity values were normalized to the total cell number in each well as assessed by a trypan blue staining assay.

For ICAM-1 labelling, Ea.hy926 cells were activated with TNF-α (2000 U/mL, 3 hours)⁹ in serum deficient DMEM to stimulate the expression of ICAM-1 on the cell surface before polymer attachment. To account for differences in reactivity between Q-tagged polymers, concentrations of polymer added to the solution were adjusted using NH₃ generation data to ensure that a similar number of polymers attached to the cell surface. Following polymer attachment, the cells were fixed, and labelled with PE-Cy™5 Mouse Anti-Human CD54 (1:20, BD Biosciences™) for 20 minutes at room temp in the dark. The cell monolayers were trypsinized and subjected to flow cytometry (CytoFLEX™, Beckman Coulter™) in which 10,000 events were acquired and the median fluorescent intensity in the 667 channel was recorded

### Confocal Microscopy Experiments

Ea.hy926 endothelial cells were plated at a concentration of 10,000 cells/well and cultured on IBIDI μ-Slide 8 well chamber slides in DMEM (10% FBS, 1% P/S) for 7 days. Ea.hy926 cells were washed three times with DPBS and stained with CellMask™ deep red plasma membrane stain (1:1000× dilution) for 15 minutes at 37° C., 5% CO₂. Cells were washed three times with cold DPBS and treated with 1 mM LPG-Q (containing 10% BODIPY FL) for 30 min at 4° C. in UW solution fortified with 3 mM GSH, 5 mM CaCl₂, 0.2 U/mL gtTGase. Following polymer attachment, cells were washed twice in DPBS, exchanged into phenol-free DMEM (5% FBS, 1% P/S) and immediately imaged. All confocal images were acquired using an inverted Zeiss Axiovert 200M™ spinning disk confocal microscope equipped with a QuantEM 512SC Photometrics™ camera (512×512 pixels size) and an incubator platform for live-cell imaging. Images were captured in series using a 60× Oil Plan-Fluor objective lens coupled to a spherical aberration correction unit. Z-stacks were acquired in 0.2 μm increments using the 640 and 488 laser channels and Cy5 and FITC bandpass emission filters. All experiments were repeated three times (3 independent experiments) and the results were pooled. Images of live cells were acquired within one hour of staining.

Enzyme-mediated glycocalyx removal from endothelial surface

Ea.hy926 endothelial cells were treated with 0.5 mM LPG-SS or LPG-Q (containing 10% BODIPY FL) for 30 min at 4° C. in UW solution fortified with 3 mM GSH, 5 mM CaCl₂, 0.2 U/mL gtTGase. Following incubation, monolayers were washed twice with room temperature DPBS then incubated with serum starved cell media containing 0.1 mM H₂O₂ and 1 nM epinephrine for 1 hour at 37° C. according to previous procedures.¹⁰ The stimulation of the cells with reactive oxygen species (H₂O₂) and catecholamines (epinephrine) has been demonstrated to upregulate the expression of glycocalyx degrading extracellular proteases including human matrix metalloprotease's (MMP). Cells were washed, trypsinized and immediately subjected to flow cytometry. Controls with no enzyme treatment were used to set gates for 100% labeled cell populations.

PBMC adhesion to endothelial surfaces

Confluent Ea.hy926 monolayers seeded in a 48 well plate (30,000 cells/well) were activated with TNF-α (2000 U/mL, 3 hours). To obtain PBMCs, EDTA anticoagulated blood from healthy donors was subjected to ficoll density gradient centrifugation Histopaque™-1077 (Sigma Aldrich™). PBMCs were cultured for 24 h in RPMI-1640 containing 10% FBS (v/v), 1000 U/mL recombinant human IL-2 (Cedarlane™)¹¹ and 1% P/S. Prior to fluorescent labelling, cultured PBMCs were tested for absence of platelets by labelling an aliquot of cells with FITC-labelled anti-human CD45 (1:200 Beckman Coulter™) and analyzed by flow cytometry (CytoFLEX™, Beckman Coulter™). PBMCs containing <1% platelets were implemented for further study. The PBMC composition was also measured using a hematological analyzer (XN-550™ automated hematology analyzer, Sysmex Canada Inc.™). Following IL-2 treatment the typical composition of the PBMCs were ˜80% lymphocytes, 5% immature granulocytes, 2% basophils and 13% monocytes. PBMCs were then suspended in Ca²⁺ and Mg²⁺ free PBS (1×10⁶ cells/mL), labelled with 5 μM CellTracker™ Green (CTG) CMFDA Dye (Invitrogen™) for 30 minutes at 37° C., washed twice with PBS, and resuspended in binding buffer (HBSS containing 2 mM CaCl₂, 2 mM MgCl₂, 1% BSA) to a final concentration of 2.5×10⁶ cells/mL. PBMCs were then slowly added to activated monolayers (with or without treatment with 0.5 mM LPG-Q or 0.56 mM LPG-Q-Sia3Lac) to a final concentration of 0.75×10⁶ cells/mL and left to incubate for 1 hour at 37° C. Following PBMC attachment, Ea.hy 926 monolayers were washed with PBS and fixed with 4% PFA for 15 minutes at room temperature. Images were acquired using confocal microscopy at confluent regions focused on the PBMC containing plane in confluent areas. PMBC adhesion was quantified by lysing the cells with RIPA buffer, spinning at 15,000 g to remove cell debris and measuring the fluorescent intensity of the CMFDA Dye in the lysate (λₑₓ/λₑₘ=492/517 nm using a plate reader (Spectramax3™, Molecular Devices™).

PBMC-mediated cytotoxicity

EA.hy 926 monolayers were activated using TNF-α and PBMCs were activated using IL-2 as described in PBMC adhesion experiments. Following activation, PBMC was collected and resuspended in DMEM (Phenol free, 5% FBS, pen/strep). PBMCs were then added to activated EA.hy 926 monolayers (modified and unmodified) to a final concentration of 0.15×10⁶ cells/mL and left to incubate at 37° C. and 5% CO₂ for 18 hour. After treatment, the supernatant was collected and cell debris was removed through centrifugation. LDH content was measured using LDC cytotoxicity assay (Biovision Inc.™). Cytotoxicity was calculated using the equation below;

$$\% cytotoxicity = \frac{{LDH}_{sample} - {LDH}_{PBMC} - {LDH}_{alive}}{{LDH}_{dead}}$$

where, LDHₛₐₘₚₗₑ is the amount of LDH released from the sample, LDH_{PBMC} is the amount of LDH released from PBMC samples not subjected to EA.hy 926 monolayers, LDHₐₗᵢᵥₑ is the amount of LDH released from EA.hy 926 monolayers not subjected to PBMCs, and LDH_{dead} is the amount of LDH released of EA.hy926 monolayers treated with RIPA buffer for 10 minutes at room temperature.
NK cell/macrophage/CD8+ T-cell depletion in PBMC isolates

Following PBMC isolation from whole blood as described previously, IL-2 activated PBMCs were selectively depleted for NK cells by immunomagnetic separation using an EasySep™ isolation kit (STEMCELL Technologies Inc.™) through positive selection for CD56+ cells. NK content in depleted and non-depleted populations was assessed through flow cytometry (Cytoflex™, Beckman Coulter™) by labeling lymphocyte populations with both a general Anti-Human CD45 (FITC conjugated, BD Biosciences™, 1:40 dilution) and Anti Human Siglec-7™ (APC conjugated, Biolegend™, 1:20 dilution). CD8+ T-cells were depleted from IL-2 activated PBMCs through positive selection using Anti-Human CD8+ Dynabeads™ (Thermo Scientific™) CD8+ T-cell content in depleted and non-depleted populations was assessed through flow cytometry (Cytoflex™, Beckman Coulter™) by labeling lymphocyte populations with Anti-Human CD8a (FITC conjugated, STEMCELL Technologies Inc.™, 1:40 dilution). To generate macrophage/monocyte depleted PBMC populations, isolated PBMCs were incubated in serum-starved RPMI media for 3 hours and non-adherent cells were removed and subsequently, subjected to IL-2 activation. After the extent of depletion was quantified, Ea.hy926 cells were subject to cytotoxicity assays. The amount of PBMCs added to the target cells was calculated to account for NK, T-cell or macrophage/monocyte depletion. For example, PBMC populations containing 20% NK cells (population A) were added to target cells in a 10:1 E:T ratio (6.1×10⁵ cells/well). PBMCs isolated from the same donor and depleted for NK cells (population B) were added to target cells at a quantity of 4.8×10⁵ cells/well which is the number of non-NK cells present in cytotoxicity studies using population A PBMCs. As such, the impact of non-NK cytotoxicity is normalized for both populations A and B.

Co-immunoprecipitation and western blot analysis

Following Ea.hy926/PBMC co-culturing, PBMC containing supernatants were removed, pelleted (300 g, 5 minutes) and subjected to immunomagnetic separation to collect NK cells. Immunomagnetic separation of NK cells was carried out using an EasySep™ isolation kits (STEMCELL Technologies Inc.™) through positive selection for CD56+ cells. Following isolation, cell suspensions were lysed with RIPA buffer containing Halt™ protease inhibitor (Thermo Scientific™) for 10 minutes at ambient temperature and cell debris was removed through centrifugation (15 minutes at 15,000×g and 4° C.). A BCA assay was used to quantify protein concentration and equal amounts of proteins were reduced by boiling in 4×SDS loading dye with 2.5% β-mercaptoethanol. For western blotting, proteins were resolved by SDS-PAGE using a 4-20% gradient gel (BioRad™) and transferred to nitrocellulose by wet transfer (Tris-glycine, 20% MeOH) at 95 V for 2 h. Blocking and antibody incubation conditions were conducted in 1× Dulbecco's phosphate-buffered saline with 0.05% Tween-20 (PBST). Blots were blocked in PBST with 5% w/v nonfat dry milk powder and probed with anti-SHP-1 mAb (R&D Systems™, 1:400 dilution) followed by rabbit anti-goat Dylight 800™ secondary antibody (1:10,000 dilution) in 3% BSA/PBST. Membranes were then scanned for fluorescence by an Odyssey 9410™ imaging system (Leica™) and results were normalized to glyceraldehyde 3-phosphate dehydrogenase (GAPDH) expression.

Cytokine profiling and assessment of CD8+ T-cell activation

PBMC-containing supernatants from Ea.hy 926/PBMC co-cultures were removed and pelleted (300 g, 5 minutes). The resulting supernatant was diluted 4-fold with PBS containing 1% BSA. Then, IL-10 or IL-6 content was quantified using a Human IL-10 DuoSet ELISA Kit™ (R&D Systems™) and a Human IL-6 Quantikine ELISA Kit™, respectively. All studies were carried out according to the manufacturer's protocols. For T-cell activation, PBMC pellets were resuspended in PBS containing 1% BSA to a final concentration of 2×10⁶ cells/mL and treated concurrently with APC-conjugated anti-CD8a mAb (BD Biosciences™, 1:40 dilution) and FITC-conjugated anti-CD69 mAb (BD Biosciences, 1:40 dilution) for 20 minutes at ambient temperature. Flow cytometry profiles were acquired using a 3-laser Cytoflex™ flow cytometer from Beckman Coulter Life Sciences™ (10,000 events).

Assessment of endothelial cytotoxicity imparted by cytotoxic T cells

The experiment was performed similar to previously described PBMC/Ea.hy926 co-culture experiments by replacing the effector with CAR-T cells. Ea.hy926 cells were engineered with polymers in UW solution as outlined. Cryopreserved CAR T cells were thawed using standard protocol41 and were pelleted and resuspended in phenol-free DMEM (5% FBS, pen/strep) at an effector (CAR-T cell) to target (Ea.hy926) ratios of 2:1, 1:2 and 1:10 (Vf=100 μL). After 18 hours, supernatants were removed from the wells and PBMCs were removed through centrifugation (300 g×5 minutes) and measured for LDH content to probe cell lysis using an LDH cytotoxicity assay (Biovision Inc.™). CAR-T cell-mediated cytotoxicity was calculated according to Eq. 2 replacing PBMC LDH release with CAR-T cell LDH release. LDH released by Ea.hy926 following 18 hour culture period that have not undergone polymer attachment and cultured in the absence of CAR-T cell is the 100% alive control and LDH released by Ea.hy926 following treatment with RIPA buffer for 10 minutes at ambient temperature is 100% dead control.

### Animals

C57BL/6J and Balb/c mice were obtained from Jackson Laboratories™ (Bar Harbor, Maine), bred and used for experimentation at 8 to 12 weeks of age. The mice were maintained at the animal facility of the Simon Fraser University (Vancouver, BC, CA) or the Center for Comparative Medicine of Northwestern Univeristy (Chicago, IL, USA). All protocols used in this study were reviewed and approved by the Simon Fraser University Animal Care Committee following the guidelines set out by the Canadian Council on Animal Care or the Institutional Animal Care and Use Committee of Northwestern University.

### Murine Aortic Interposition Grafting

Murine aortic interposition grafting was performed as described previously.^{13,14} Segments of abdominal aorta from Balb/c (H2d) donor mice were excised, flushed with UW solution and then incubated in UW solution alone (containing 5 Mm CaCl₂, 0.2 U/Ml gTGase and 3 mM GSH) or UW solution fortified with 0.2 U/mL gtTGase and 0.5 Mm polymer (LPG-Q, LPG-QSia3Lac) for 1 hour at 4° C. Aortic segments were then flushed with saline and interposed into the resected infra-renal aorta of C57BL/6 (H2b) recipient mice.

### Morphological Analysis of Allograft Arteries

At day 2, 15 and 42 post-transplantation, grafted artery segments were perfusion fixed with 4% (v/v) paraformaldehyde, excised, and frozen in optimal cutting temperature medium. Eight-micron sections were prepared and stained with hematoxylin and eosin (H&E). Tissue sections were stained with H&E. Histological features of arterial injury and inflammation at day post-transplantation were graded in a blinded manner on a 0-4 scale that considered the amount of medial injury and leukocyte infiltration. Medial thickness was quantified using ImageJ software (NIH) as described.69 Histological features of acute rejection at day 15 post-transplantation were graded in a blinded manner on a 0-12 scale because acute rejection involves more extensive features of immune reaction and injury compared to day 2 post-transplantation. Specifically, there is extensive infiltration of the arterial media by host leukocytes that causes medial damage characterized by swelling, death of smooth muscle cells, damage to the elastic laminae, fibrin deposition in severe cases, and early development of intimal thickening. Leukocyte infiltration, medial injury and intimal thickening were each graded on a 0-4 scale and an aggregate score calculated. For evaluation of chronic rejection, intimal thickening at day 42 was quantified by averaging the luminal narrowing in 3 cross-sections per artery that were ˜100, 150 and 200 μm past the suture sites using ImageJ™ software.

### Cytokine Array Analysis

To assess the immune modulatory effect of enzymatic polymer grafting, an antibody array kit based on a sandwich ELISA principle (Proteome Profiler™, Panel A; R&D Systems™) was used to screen the cytokine and chemokine expression level in sera of animals according to the manufacturer's protocol. Relative expression of 40 murine cytokines and chemokines captured by corresponding antibodies spotted on a nitro-cellulose membrane was determined.

The antibodies for the following cytokines were spotted on the membrane: BLC, C5a, G-CSF, GM-CSF, I-309, Eotaxin, sICAM-1, IFN-g, IL-1a, IL-1b, IL-1ra, IL-2, IL-3, IL-4, IL-5, IL-6, IL-7, IL-10, IL-13, IL-12 p70, IL-16, IL-17 IL-23, IL-27, CXCL10 (IP-10), I-TAC, KC, M-CSF, CCL2 (MCP-1), CCL12 (MCP-5), MIG, CCL3 (MIP-1a), CCL4 (MIP-1b), CXCL2 (MIP-2), CCL5 (RANTES), SDF-1, TARC, TIMP-1, TNF-α, TREM-1. In brief, serum samples were collected at day 2 and 42 post-transplantation from mice after murine aortic interposition grafting and spun for 15 min at 15,000 g at room temperature (RT) to remove debris. Following the blocking step for 1 h at 4° C., a mixture of detection antibody cocktail and 100 μL of serum sample was incubated overnight at 4° C. with the membrane. Captured proteins were labeled with a streptavidin-functionalized IR-Dye (IRDye 680RD Streptavidin; LI-COR) (1:20,000 dilution in 10% bovine serum albumin, 30 min at RT) and scanned using a LI-COR Odyssey Infrared Imaging System™. The software ImageJ™ (Version 1.52s) was used to determine the integrated density of the spots on the array images after background subtraction.

To quantify donor specific antibodies, sera were collected from transplant mice at 42 days post-transplant, serially diluted, and incubated with splenocytes from BALB/c mice. This was followed by staining with a polyclonal Goat-anti-mouse secondary-FITC antibody (BioLegend™) and staining for CD3-PE (17A2, BD Biosciences™). DSA reactivity on CD3-positive cells was analyzed via flow cytometry using a BD LSRFortessa X-20™ (BD Biosciences™). Data is presented as the MFI of DSA reactivity on CD3-positive cells.

Murine skin grafting of aortic interposition grafted mice

Murine skin grafting was performed as described previously.^{75,76} Donor mice were anesthesized and the back hair shaved, depilated and cleaned using iodopovidone and alcohol wipes. The mice were then euthanized and the skin harvested. The panniculus carnosus, subcutaneous fat and connective tissue were removed, leaving the dermal layer for grafting. 8 mm diameter skin grafts were prepared using a biopsy punch. Mice that received aortic transplants were used as skin transplant recipients 28 days after artery transplantation. Skin graft recipients were fully anesthetized and the hair from the neck to the hip at the back shaved, depilated and cleaned. The area was subsequently prepared using iodopovidone followed by alcohol wipe. A 6 mm biopsy punch was used to carefully resect the skin at three different areas (minimum 0.5 mm apart) while preserving the underlying panniculus carnosus layer of the hypodermis. The donor grafts were positioned and then secured onto the resected areas using 7-0 Prolene™ sutures. The skin grafts were evaluated daily and rejection was determined by the physical appearance of the graft (colour, shape and texture).

### Syngeneic Murine Kidney Transplantation

Kidneys from C57BL/c mice were transplanted into syngeneic recipients by using the surgical techniques previously described by Zhang et al.¹⁷⁻¹⁹ Briefly, the donor kidney was procured along with renal vessels attached to a segment of the abdominal aorta and inferior vena cava (IVC), perfused with either UW or polymer, and cold static storage in UW or UW+LPG-Q-Sia3Lac for 4 hours prior to being transplanted into the recipients (N=5). In the recipient surgery, the kidney was flushed with saline, followed by anastomoses between donor aorta and inferior vena cava (IVC) and the recipient abdominal aorta and IVC in an end-to-side manner, respectively. To complete the ureteral reconstruction, the donor bladder patch was anastomosed the bladder dome of the recipient. Both native kidneys of the recipients were removed during the surgery. Therefore, the recipients' survival depended upon the transplanted kidney. For sham control, the age matched B6 underwent unilateral nephrectomy.

### Blood Analysis

The whole blood sample was collected from recipient mice at both day 2 and day 7 post-surgery and were used for the measurement of blood urea (BUN) and creatinine using i-STAT Bioanalyzer™ (i-STAT CHEM8+ Cartridges™)

### Histological Analysis of Graft Damage

Grafts were harvested from recipients at humane (graft failed) or experimental endpoint (at day 7 post-surgery), followed by formalin fixation and paraffin embedding. Tissue sections were stained with H&E. The tubular damage of each graft was semi-quantitatively scored as follows:²⁰ 0, no difference from a sham kidney; 1, up to one third of tubules showing cell swelling, brush border or nuclear loss; 2, as for score 1, but greater than one third but less than two third of tubular damage; and 3, greater than two third of tubular damage. Two sections from each graft were examined, and the tubular damage was scored in total 20 microscopic fields (200× magnification) in a blinded fashion. The data represented the average score of these 20 fields.

### Allogeneic Murine Kidney Transplantation

Kidney transplantation was performed similarly to the process described above; kidneys from Balb/c mice were transplanted into allogeneic B6 recipients (N=8) using the same method as described above, including organ procurement and modification.

The whole blood sample was collected from recipient mice at both day 7, day 14 and day 30 post-surgery and were used for the measurement of blood urea (BUN) and creatinine using i-STAT Bioanalyzer™ (i-STAT CHEM8+ Cartridges™)

Kidney tissues at day 30 post-transplantation were preserved by formalin-fixed and paraffin-embedded. For histological scoring of severity of cellular infiltration, tissue sections (˜4 μm thickness) were routinely stained with hematoxylin and eosin (HE). The stained tissue slides were scanned with Leica SCN400™ slide scanner (Leica Microsystems Inc.™, Concord, ON, Canada). The cellular infiltration in each high-powered field (hpf) (200 Å˜magnification) was scored as follows in a blinded fashion: 1(0%-24% of the view affected with infiltrates), 2 (25%-49%), 3 (50%-74%), and 4 (>75%). An average number of at least 20 randomly selected fields in the cortex of kidney sections represented the score of the cellular infiltration of a transplant. The mesangial expansion (ME) of the glomeruli as a marker of the transplant rejection was examined by using a semi-quantitative scoring system in Masson's trichrome (MT)-stained tissue sections. The scoring system consisted of 1 to 4 scale to determine the severity of the extracellular matrix (ECM) deposition into both the capillary walls (the incrassation of basement membrane) and the mesangium based on the percentage of the area stained strongly with MT, indicating the ME in an affected glomerulus: 1 (0%-24% of the area affected with densely stain), 2 (25%-49%), 3 (50%-74%), and 4 (>75%) (see FIG. 34). A range of 100 to 120 glomeruli were counted for each transplant, and were averaged for the severity of ME.

### Statistical and Regression Analysis

Data are expressed as the mean f 95% confidence interval using the sample standard deviation. Statistical analyses were performed using GraphPad Prism version 7.0™ software, using unpaired t-tests with Welch's correction. Samples were denoted as statistically significant. p<0.05 (*), p<0.01 (**), and p<0.001 (***). Experiments were performed in triplicate and results were pooled into a single dataset unless otherwise stated. Regression analysis using least-squares was also performed using GraphPad Prism version 7.0™ software.

### Superoxide Radical Scavenging Assay

In a 200 mL reaction, 50 mM polymer, 78 μM NADH and 25 μM NBT were mixed in 16 mM Tris-HCl, pH 8 buffer. PMS was added to achieve a final concentration of 10 M and mixed to begin the reaction. After incubating for 5 minutes, the absorbance was measured at 560 nm in a spectrophotometer. The superoxide scavenging activity was calculated using blank subtracted values with equation 1 and reported in percentage;

$$Superoxide scavenging acitvity ( \% ) = 1 - ( \frac{{A}_{sample}}{{A}_{con trol}} ) \qquad Eq . 1$$

When performed in a cell environment, the assay buffer, Tris-HCl, is replaced with DMEM solution without phenol red indicator and polymer was conjugated onto the cell surface.

Endothelial cell surface engineering with polyglycerols using guinea pig liver transglutaminase

EA.hy 926 cells plated for 15 days were washed twice with warm PBS before introducing a reaction cocktail containing 0.5 mM polymer, 5 mM CaCl₂, 3 mM DTT and 0.2 U/mL transglutaminase made in DMEM media solution without phenol red. The reaction was incubated at 4° C. for 30 minutes. Then the supernatant was removed and subjected to ammonia generation assay and the cells were washed twice with warm PBS to remove unreacted polymers. The treated cells were left to incubate at 37° C. and 5% CO₂ for 2 hours prior to further experimentation.

### Cell Viability Measurements

Cell viability measurements were performed on modified cells after washing with warm PBS twice. Then, the cells were subjected to MTS (3-(4,5-dimethylthiazol-2-yl)-5-(3-carboxymethoxyphenyl)-2-(4-sulfophenyl)-2H-tetrazolium) assay kit (Cell Titer 96 Aqueous One solution Cell Proliferation Assay™) and by following the manufacturer's protocol. All recorded values were normalized to healthy cells untreated with polymers. Platelet activation was performed by PE-labelled anti human CD62a stain was prepared by diluting 20 times using plasma. An aliquot of each platelet sample was labelled using the diluted PE-labelled anti-human CD62a solution (1:10 dilution) for 30 minutes at room temperature. Platelets incubated in 0.1 mM TRAP were used as positive control and platelets incubated in PE-labelled IgG were used for non-specific binding control. Each sample was analyzed for 10000 platelet events. Each experiment was replicated using a minimum of 3 donors and each donor was performed in triplicates. For human pancreatic islets, live/dead imaging assay was used for cell viability and DTZ detection assay was used to determine insulin-producing beta cells from mixed cell cultures.

### Glycocalyx Shedding Experiments

EA.hy 926 cells were grown to confluency. Media was replaced, washed with DPBS and incubated with 78 M NADH, 10 M PMS (unless otherwise noted) and the indicated concentration of polymer; with PMS being last to be added. The treated cells were left to incubate at 37° C. and 5% CO₂ for 30 minutes in the dark. The cells were then washed extensively, stained using WGA (5000 g/mL) for 15 minutes and harvested to be analyzed using flow cytometry. Neuraminidase enzyme was used as a positive control to shed the glycocalyx.

### Transendothelial Protein Passage Experiments

HMEC-1 cells at 60-80% confluence were trypsinized and seeded in a tissue culture inserts (3415, Corning™) with polycarbonate supports (3 μm pore size, 0.33 cm² surface area) at 100,00 cells/cm². The tissue culture inserts were placed in 24 well plates. HMEC-1 cells were allowed to grow for 1 week in tissue culture inserts for 1 week with media changed every 2 days prior to performing transendothelial protein passage experiments. The passage of FITC-labeled BSA (A92771, Sigma™) across the monolayer was tracked over 3 hours. To begin, the media in both compartments was replaced with serum-free media for 1 hour. After, 0.5 mg/mL FITC-BSA in media was added to the upper compartment and 0.5 mg/mL unlabelled BSA (A-7888, Sigma™) in media was added to the lower compartment. Every hour for 3 hours, 100 μL aliquots were collected from the lower compartment and replaced with media containing 0.5 mg/mL unlabeled BSA. The fluorescence of the collected media was recorded and the concentration was calculated against a standard curve of FITC-labeled BSA.

### Assessment of Inflammatory Response in Macrophages

THP-1 cells were cultured in THP-1 media (RPMI-1640 media supplemented with 10% FBS, 1% P/S and GlutaMAX™) at 37° C. and 5% CO₂. To induce into M1 macrophages, THP-1 cells were first induced to differentiate into M0 macrophages by culturing in M1 media (THP-1 media supplemented with 100 nM phorbol myristate acetate (PMA)) at 37° C. and 5% CO₂ for 72 hours. Then, the M0 cells were further induced in M1 cells by culturing in M1 media (THP-1 media supplemented with 100 ng/mL lipopolysaccharide (LPS) and 20 ng/mL IFN-γ) alone or supplemented with polymers at 37° C. and 5% CO₂. After 24 hours, the supernatant was collected and diluted 1:8 prior to the quantification of TNF-α released using a TNF-α ELISA Kit (Invitrogen™, 88-7346). The ELISA was used according to the manufacturer's protocol.

### Glycocalyx Quantification Experiment

Ea.hy926 cells at 60-80% confluence were trypsinized and plated in a 48 well plate at 30,000 cells/well. Cells reached 100% confluence at day 3 and allowed to grow for a further 12 days at 37° C. and 5% CO₂. Cells were washed twice with cold PBS and subjected to various conditions. CSE and TNF-a treatment were performed as described previously. After subjecting the cells to the various conditions, the cells were then washed extensively, labelled with WGA-FITC (5000 μg/mL) for 15 minutes at room temperature in the dark. The cell monolayers were trypsinized and subjected to flow cytometry

### EXAMPLES

### Example 1: Engineering the Endothelial Glycocalyx with Glycopolymers Under Cold Storage Conditions

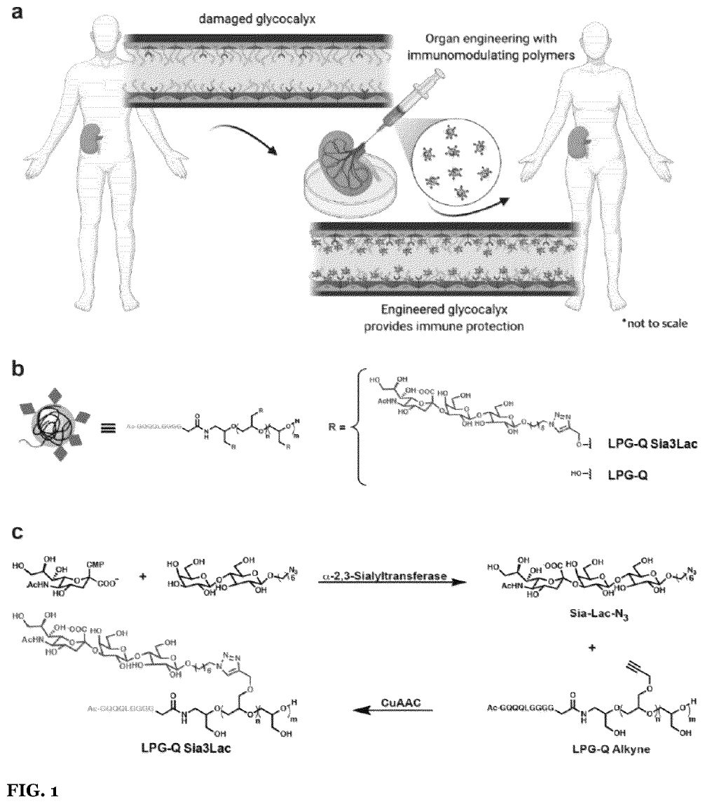

To enable tissue engineering under cold storage conditions, we chose to attach functionalized polymers to the endothelial surface through an enzyme-mediated conjugation strategy using guinea pig liver tissue transglutaminase (gtTGase). This enzyme forms an irreversible isopeptide bond between amine donors (e.g. lysine residues) on the cell surface and glutamine-functionalized (Q-tagged) polymers. The polymer scaffold in some embodiments might be highly hydrophilic, biocompatible and non-immunogenic, and possess a suitable architecture for multivalent presentation of bioactive groups. Linear polyglycerol (LPG), with its multiple free hydroxyl groups, was selected as the scaffold polymer for the current application.³⁹ LPG scaffolds were prepared with clickable alkyne handles (LPG-alkyne) and a gtTGase-reactive Q-tag (Ac-GQQQLGGGGG) (LPG-Q) (FIGS. 1b, 1c and FIG. 8 (Scheme 1)).

Below are two new sialic acid intermediates for polymer modification.

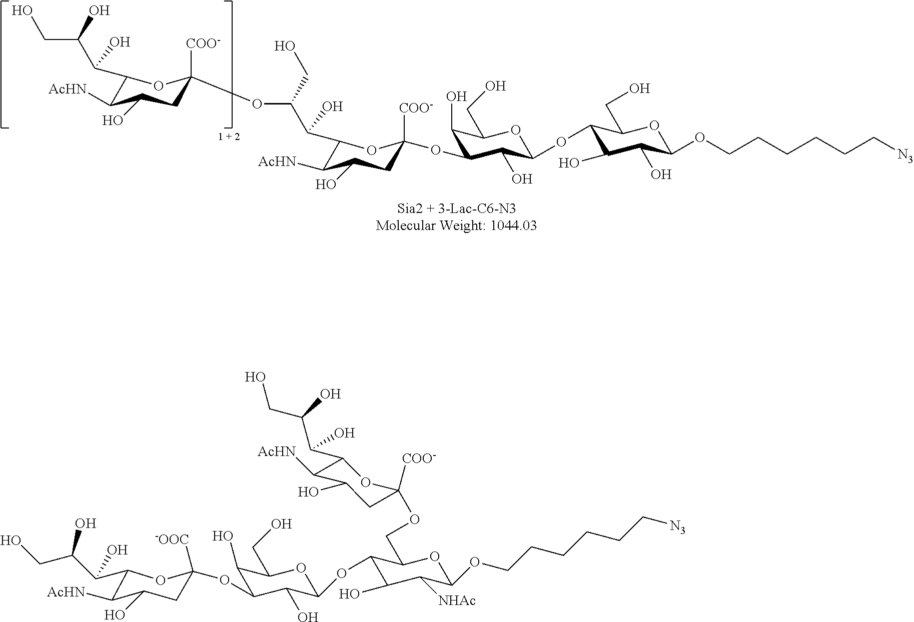

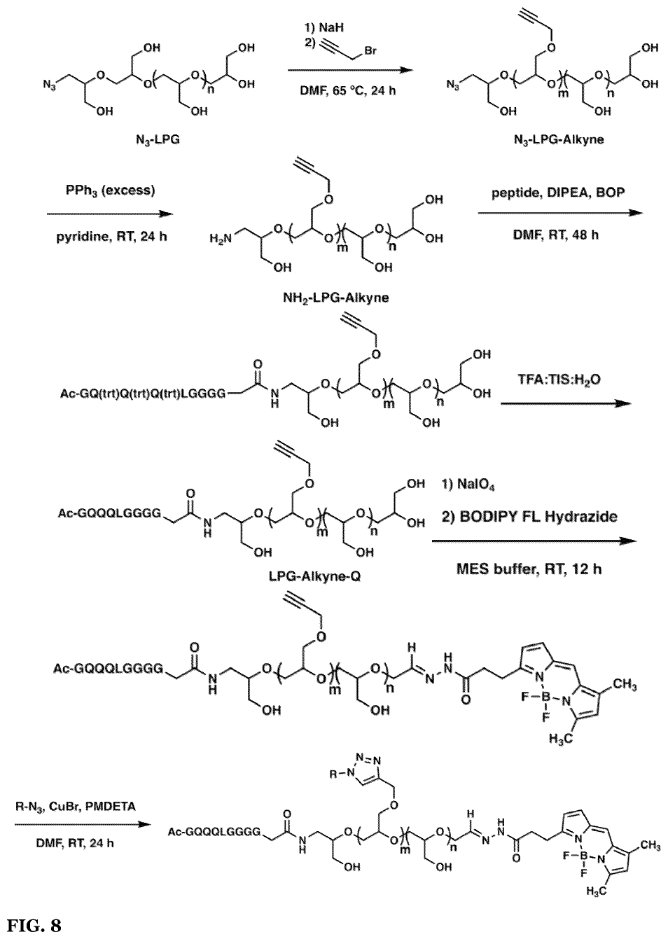

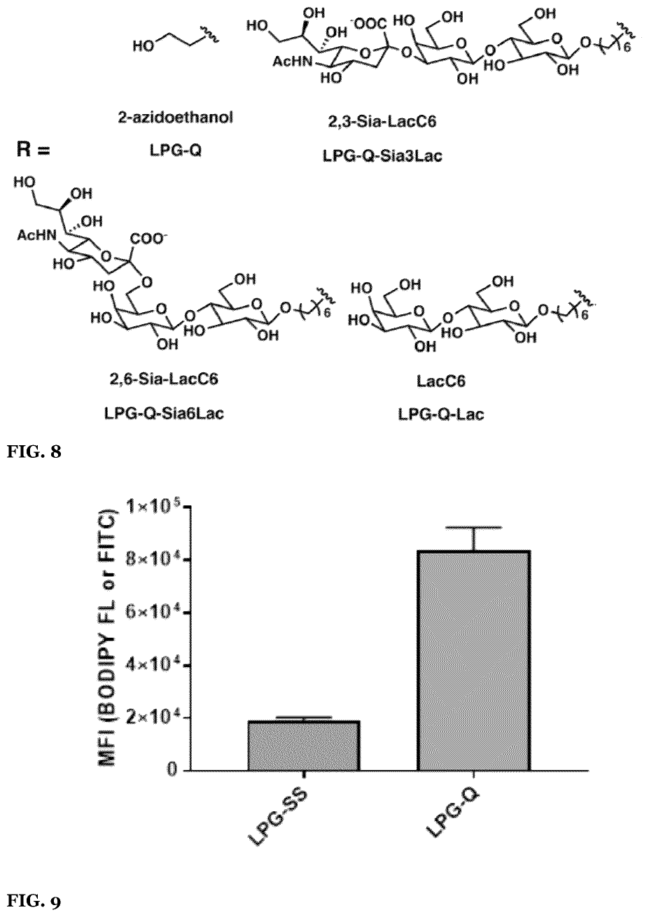

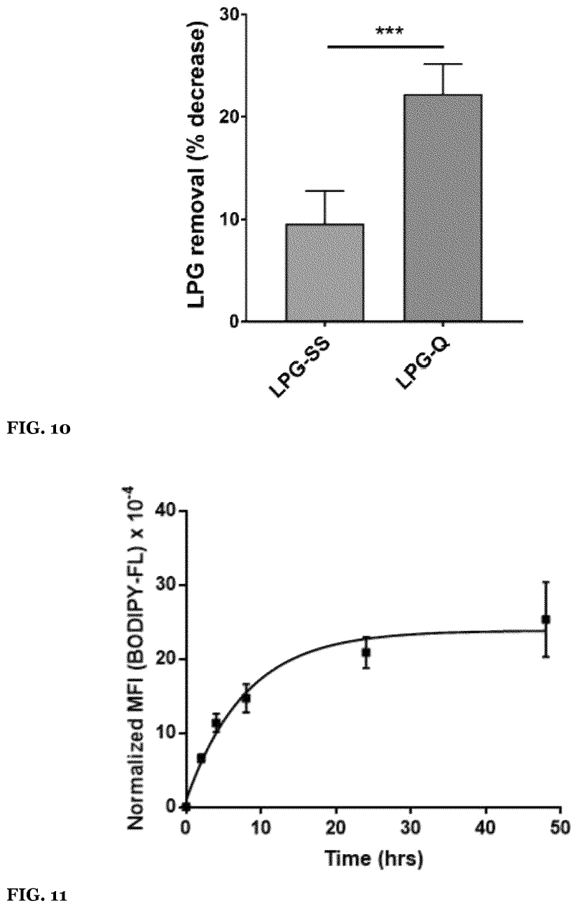

Our engineering approach under SCS requires fast reaction times at low temperatures (4° C.) in the presence of other nucleophiles in the organ preservation solution (e.g., reduced glutathione in UW solution). We first validated the benefit of a gtTGase-mediated tissue engineering approach compared to other bioconjugation strategies that are more commonly used in CSE. Under similar conditions, the enzyme-mediated approach produced ˜4.4-fold higher levels of cell-surface labelling on endothelial cells (Ea.hy926 cells) compared to the standard succinimidyl succinate amine coupling approach (FIG. 9). Enzyme-mediated grafting of LPG-Q to existing glycocalyx structures also provides higher specificity for glycocalyx modification than does chemical ligation as shown in FIG. 10.

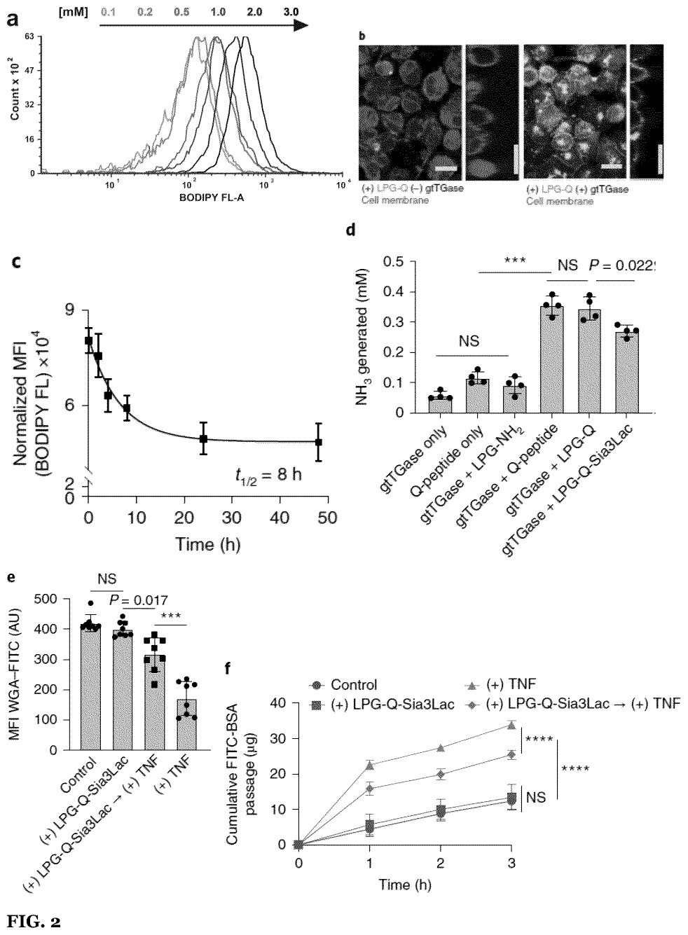

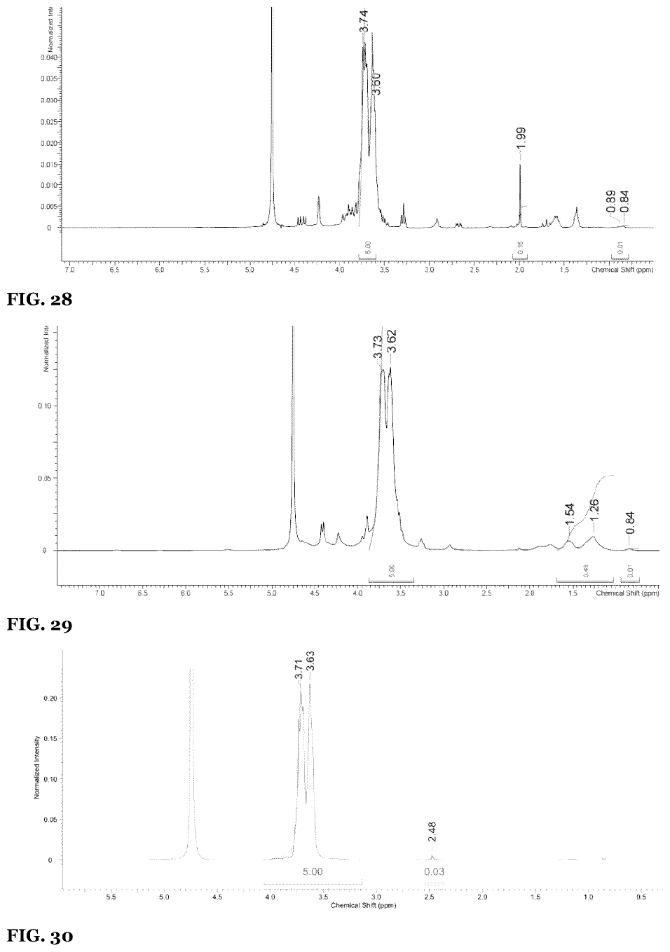

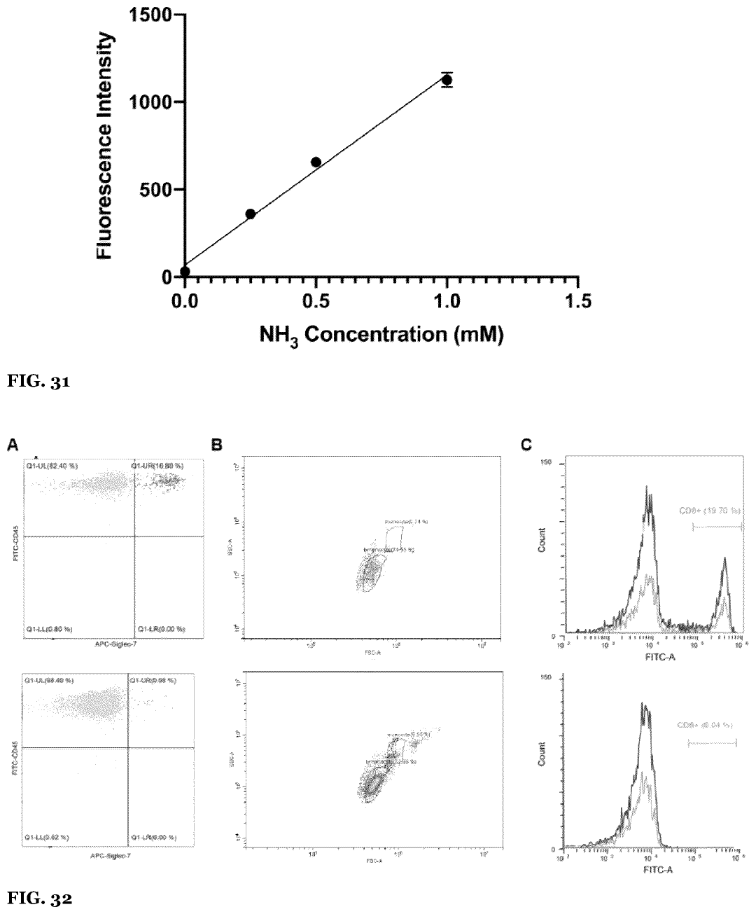

We next probed the extent of in vitro reactivity on the Ea.hy926 cell surface using various concentrations of LPG-Q (0.2 mM-3.0 mM; 10% BODIPY FL-labelled) under cold storage conditions (4° C.) for 30 minutes in UW solution fortified with gtTGase (0.2 U/mL, 3 mM GSH, 5 mM CaCl₂). A dose-dependent increase in LPG-Q attachment was observed up to 3.0 mM (FIG. 2a) and confocal microscopy confirmed the binding of polymers to the cell surface (FIG. 2b). Following rewarming (37° C.), the grafted LPG-Q was shown to have a half-life (t_{1/2) of ˜}8 hours on the cell surface (FIG. 2c) and was removed presumably by glycocalyx shedding into the surrounding media (FIG. 17) rather than internalization. Further, to measure the extent of the reaction between Q-tags and lysine substrates on glycocalyx, the production of ammonia (NH₃), a side product of transglutaminase-mediated ligation, was assessed (FIG. 2d; quantified using the curve of FIG. 31).

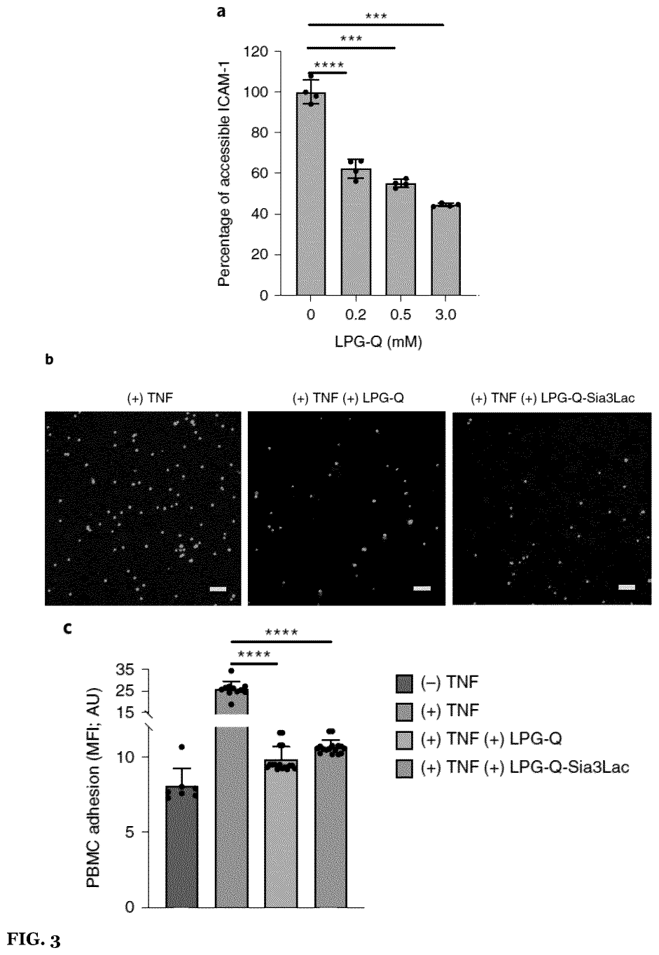

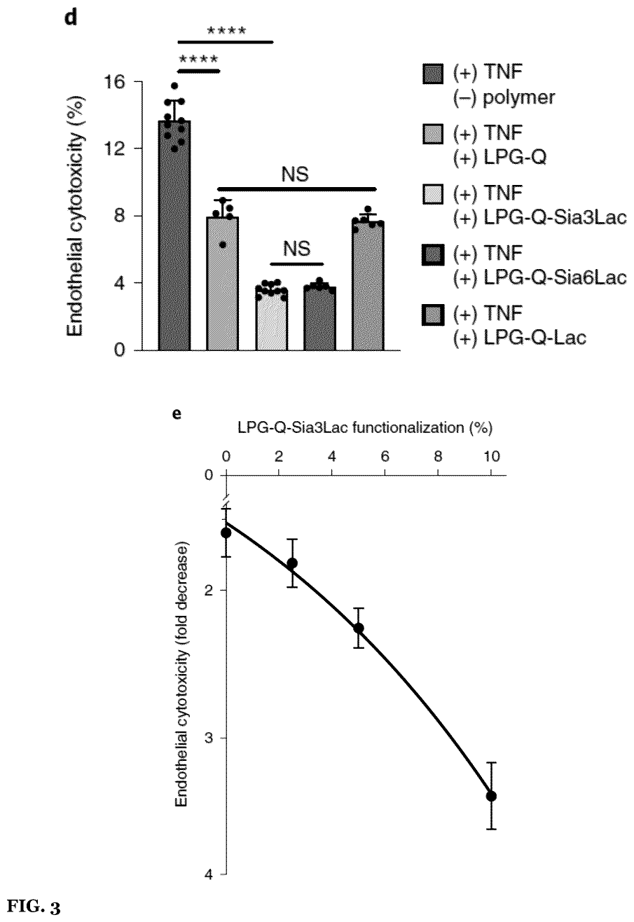

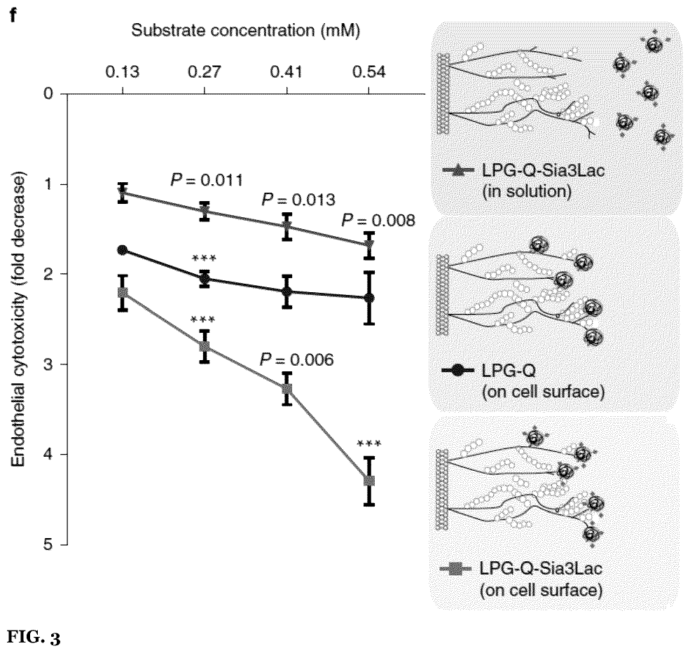

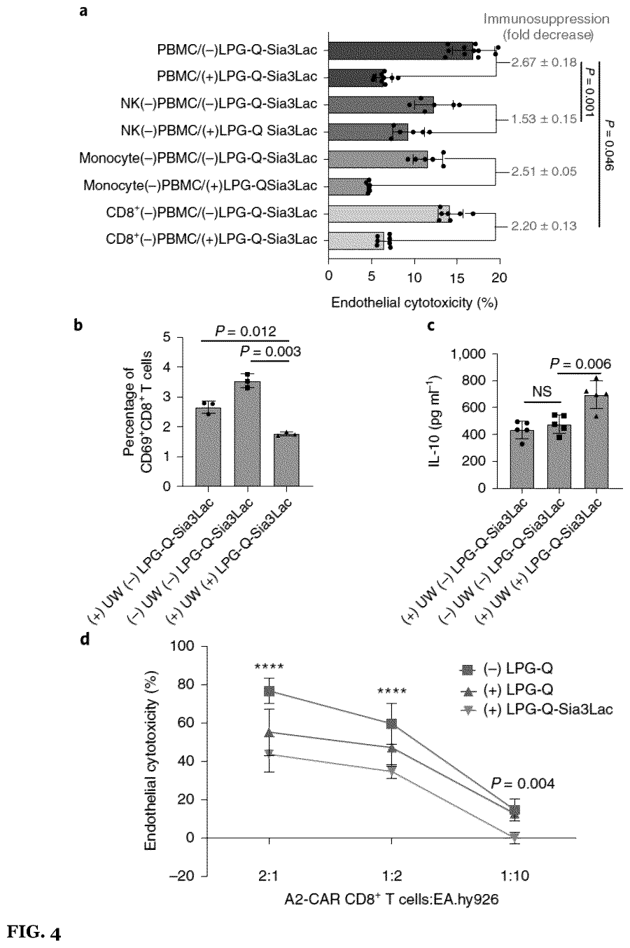

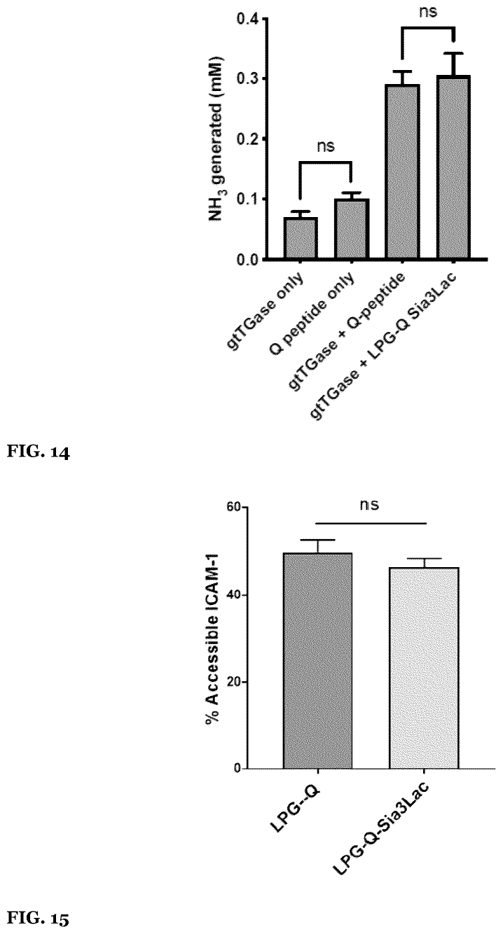

To further improve performance of the polymer, we functionalized LPG-Q with sialyl lactose to generate a glycocalyx mimic.^{34,35} Sialic acid exerts broad ranging immunosuppressive functions by binding to Siglec (sialic acid-binding, immunoglobulin (Ig)-like lectin) immunosuppressive receptors, expressed on immune cells such as NK cells, monocytes, dendritic cells and T cells. Cu(I) catalyzed azide-alkyne cycloaddition (CuAAc) afforded the sialyl lactose glycopolymers (LPG-Q-Sia3Lac, ˜17×2,3 sialyl oligosaccharides per LPG) (FIG. 1b-c and FIG. 8). gtTGase-mediated CSE of Ea.hy926 cells generated bioactive glycopolymer-modified glycocalyx (FIG. 2d); the amount of LPG-Q-Sia3Lac added was adjusted to match the similar number of LPG-Q molecules attached to the cell surface (as assessed through total NH₃ generated by each polymer). The CSE approach also worked for other endothelial cells (HMEC-1 and HUVEC cells) under the conditions studied (FIG. 13, FIG. 14). The modification of Ea.hy926 cells with LPG-Q-Sia3Lac did not alter the glycocalyx composition as measured by WGA staining (glycocalyx stain; FIG. 2e). Treatment with LPG-Q-Sia3Lac prior to treatment with TNF-α resulted in increased preservation of the glycocalyx as the amount of glycocalyx present on the cell surface was higher on CSE treated cells compared to controls following TNF-α induced glycocalyx shedding. Barrier functionality of Ea.hy926 cell monolayer after LPG-Q-Sia3Lac modification was measured by the diffusion of fluorescently labeled albumin (FIG. 2f) (additional functional measurements are shown in FIGS. 3 and 4); the data show that CSE with LPG-Q-Sia3Lac did not alter the baseline permeability of the monolayer to macromolecules. TNF-α treatment increased permeability in both treated and untreated groups. However, the CSE treatment decreased permeability in response to inflammatory activation by TNF-α, suggesting that the CSE with LPG-Q-Sia3Lac decreased TNF-α-induced barrier damage and further supports protection of the innate glycocalyx structure as demonstrated in FIG. 2e. Further, our RNA sequencing (RNA-seq) analysis provided evidence that modification of endothelial cells with LPG-Q-Sia3Lac did not produce significant changes in gene expression indicating non-toxicity.

### Example 2: Engineering Endothelial Cells with Bioactive Glycopolymers Evades Immune-Mediated Damage In Vitro

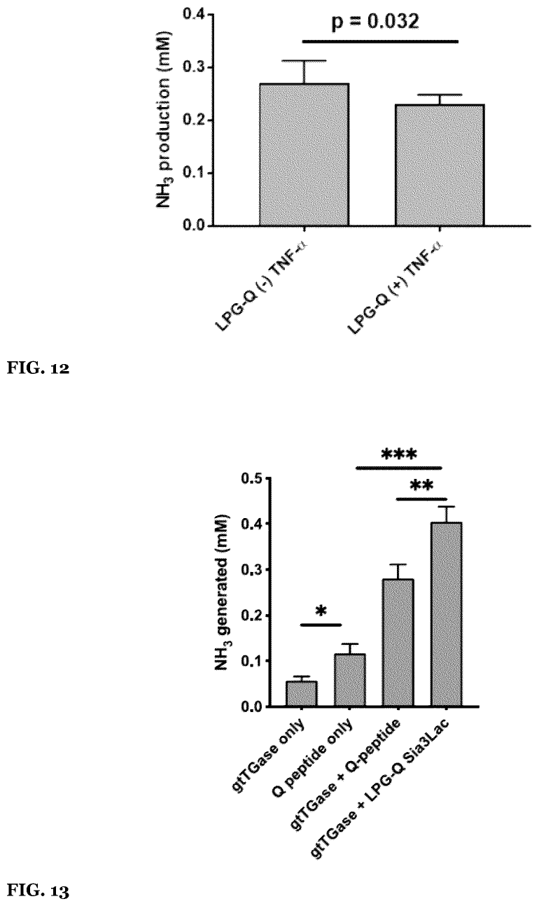

To probe the efficacy of our CSE technique in protecting endothelial cells from immune-mediated damage, we studied its ability to mask cell-surface ICAM-1, an intercellular adhesion molecule that is upregulated on the endothelium by TNF-α during inflammation, as well as its ability to impact leukocyte adhesion. Monolayers of endothelial cells, Ea.hy926, were activated with TNF-alpha and modified with increasing concentrations of LPG-Q or LPG-Q-Sia3Lac (0.2-3.0 mM) in UW fortified solutions at 4° C. The accessibility of ICAM-1 was assessed by flow cytometry (FIG. 3a, FIG. 12, and FIG. 15). CSE with LPG-Q or LPG-Q-Sia3Lac reduced the detection of ICAM-1 on the surface of endothelial cells. The extent of immunomodulation on engineered endothelial cells was further examined by measuring the adhesion of human peripheral blood mononuclear cells (PBMCs) that were activated with IL-2 (activates NK and T cells) to endothelial cells. PBMC adhesion to endothelial cells was significantly reduced by ˜2.8-fold and ˜2.4-fold by LPG-Q and LPG-Q-Sia3Lac, respectively, compared to the control (FIGS. 3b and 3c). This demonstrates the effective re-establishment of the shielding effect of healthy/functional glycocalyx structures against antibody and immune cell binding.

Next, polymer engineered endothelial surfaces were assessed for suppression of PBMC-mediated cytotoxicity. Since sialic acid is a known receptor for sialic acid-binding immunoglobulin-type lectins (siglecs) on lymphocytes, and mediates immunosuppression, the rebuilding of sialic acid-containing glycocalyx on endothelial cells after TNF-alpha treatment is anticipated to be actively immunosuppressive. PBMCs were activated with IL-2 and Ea.hy926 cells with TNF-α. Following an 18-hour co-culture using an effector-to-target ratio (PBMC: Ea.hy926) of 10:1, endothelial lysis was assessed through a lactate dehydrogenase (LDH) viability assay. Following treatment, PBMC-mediated cytotoxicity was suppressed in polymer engineered cells, with a ˜3.5-fold decrease in cell death being seen compared to the control co-cultures with unmodified cells (FIG. 3d).

We further probed the immunosuppressive role of bioactive glycopolymers displaying different carbohydrate moieties (α2,3-SiaLacNAc (LPG-Q-Sia3Lac), α2,6-SiaLacNAc (LPG-Q-Sia6Lac) or lactose (LPG-Q-Lac)). While polymer-mediated immunocamouflage alone (with LPG-Q-Lac, LPG-Q) provided just under 2-fold attenuation of PBMC-mediated cytotoxicity, the presence of sialic acids caused a further ˜2.2-fold decrease (FIG. 3d). This effect was not seen with LPG-Q-Lac, confirming that the additional immunosuppressive effect following sialylation is not simply from the increased bulkiness of the polymer following glycosylation. No difference in immunosuppression between the α2,3-SiaLacNAc and α2,6-SiaLacNAc glycopolymers was observed under these conditions. Repeating this experiment using glycopolymers displaying varying densities of α2,3-SiaLacNAc further indicated that PBMC-mediated endothelial toxicity suppression is dependent on the amount of α2,3-SiaLacNAc on the polymer scaffold (FIG. 3e).

We next investigated the influence of immobilizing sialic acid onto the cell surface in a multivalent fashion and assessed its immunosuppressive potential in comparison to its activity in solution. In nature, multiple sialic acids are often presented on a glycoprotein or glycolipid leading to strong immunosuppressive bioactivity.⁴⁵ Thus multivalent presentation of sialic acids on the cell surface may impart additional advantages. Accordingly, we profiled the PBMC-mediated cytotoxicity of glycopolymer-modified endothelial cells in comparison to control polymers both on the cell surface and in solution (FIG. 3f). While the attachment of LPG-Q to the cell surface prevented PBMC-mediated cell death, no continued significant increase in immunosuppression was observed beyond 0.27 mM. The introduction of α2,3-SiaLacNAc onto the LPG scaffold, however, significantly enhanced immunosuppression in a dose-dependent manner both in solution and on the cell surface. Though the glycopolymers are capable of multivalent binding both in solution and on the cell surface, the localization of α2,3-SiaLacNAc residues through CSE proved to be substantially more immune suppressive than the glycopolymer in solution. Engineering the cell surface with LPG-Q alone proved to elicit more potent immunosuppression than LPG-Q-Sia3Lac in solution, illustrating the powerful effect that engineering the cell surface can have on cell behavior.

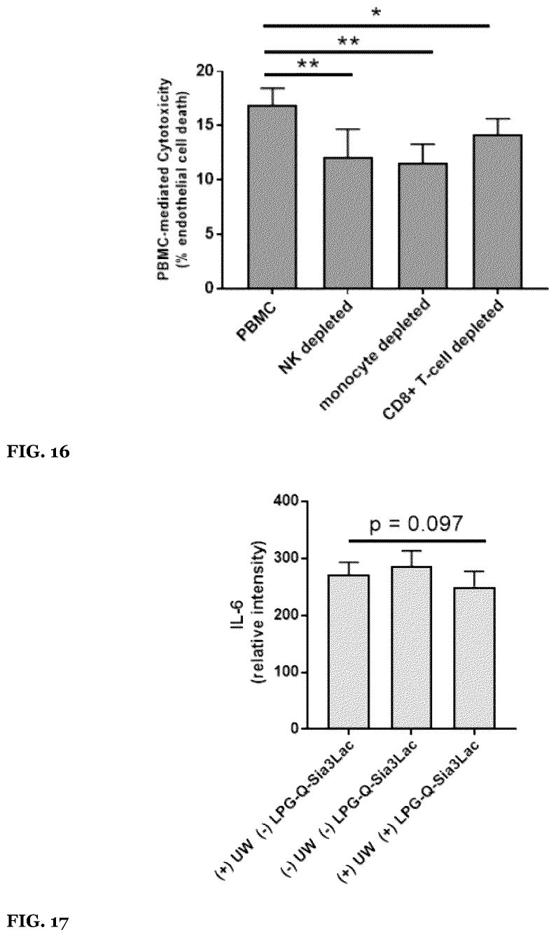

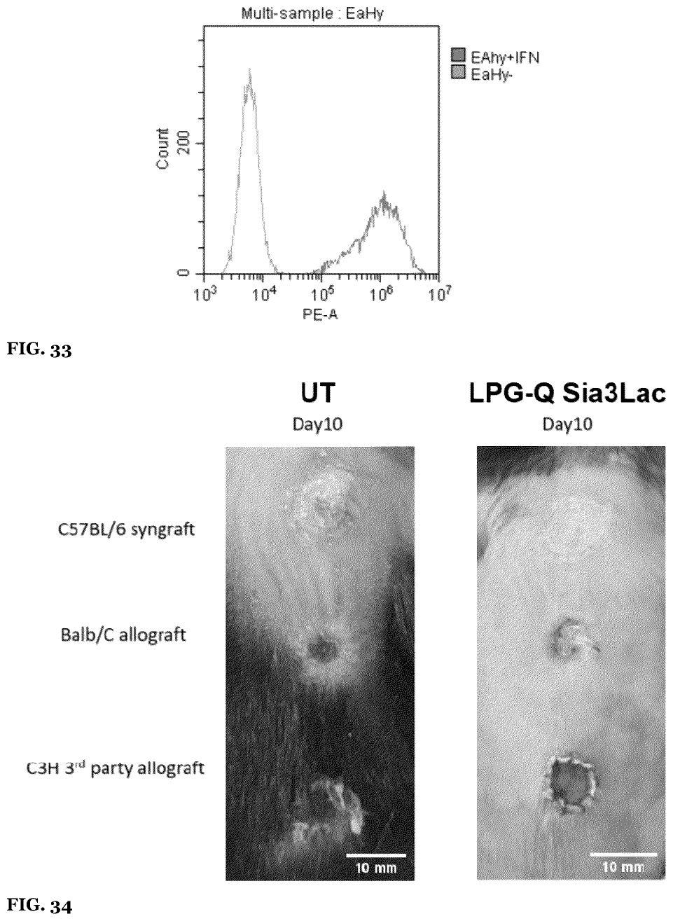

The PBMC-mediated cytotoxicity that we have examined involves multiple cell types. NK cells, monocytes and activated CD8+ T-cells can induce endothelial cell death via the release of inflammatory cytokines and direct cell lysis.^{34,35} We sought to characterize the immunoregulatory role of polymer-mediated endothelial CSE on these leukocyte types. PBMCs were depleted of either NK cells, CD8+ T-cells or monocyte/macrophage populations (FIG. 32) and then assessed for endothelial cytotoxicity on control and surface-engineered endothelial cells. The removal of each of these cell components significantly and partially attenuated lysis of unmodified cells (FIG. 16). In CSE-modified endothelial cells, inhibition of cytotoxicity by LPG-Q-Sia3Lac remained only when monocytes or CD8+ T-cells were removed. This approach did not reduce PBMC-mediated cytotoxicity when NK cells were removed (p=0.0013), indicating that the protective effect of LPG-Q-Sia3Lac mainly involved inhibition of NK cell-mediated lysis in this scenario and that endothelial surfaces modified with sialic acid containing polymers can trigger immunosuppressive pathways associated with NK cells in these experiments (FIG. 4a). We also explored the impact of endothelial CSE on CD8⁺ T-cells within the PBMC milieu. Following endothelial modification with LPG-Q-Sia3Lac, a decrease in the population of CD8⁺ T-cells that expressed the early activation marker CD69 was observed, indicating that LPG-Q-Sia3Lac reduced the number of activated CD8⁺ T-cells (FIG. 4b). Further immune suppression by our CSE approach was probed by quantifying cytokine expression. Following co-culture, IL-6 and IL-10 were secreted from cells, both with and without endothelial CSE. A robust and significant increase in IL-10 expression was observed when endothelial cells were modified with LPG-Q-Sia3Lac (FIG. 4c) while the expression of IL-6 remained constant in both treated and control samples (FIG. 17). The increased expression of IL-10 further supports the immunosuppressive effect of LPG-Q-Sia3Lac-modified cell surface. The effect of CSE in preventing endothelial cell lysis by CD8+ T cells was then examined using chimeric antigen receptor (CAR) T cells expressing a CAR that recognizes human leukocyte antigen-A2 (HLA-A2) on the surface of target cells.⁴¹ Ea.hy926 cells express HLA-A2 (FIG. 33). CAR T cells expressing HLA-A2 were generated from two donors, and the protective effect of LPG-Q-Sia3Lac on endothelial surface were analyzed. Though LPG-Q reduced endothelial cytotoxicity, the incorporation of α2,3-SiaLacNAc into the polymer allowed substantial evasion of CAR-T cell induced endothelial cell cytotoxicity (FIG. 4d). At 1:10 effector: target cells, the CSE using LPG-Q-Sia3Lac offered quantitative protection against these highly cytotoxic T cells.

### Example 3: Engineering Vascular Endothelial Cells with Immunosuppressive Glycopolymers Prevents Graft Rejection In Vivo

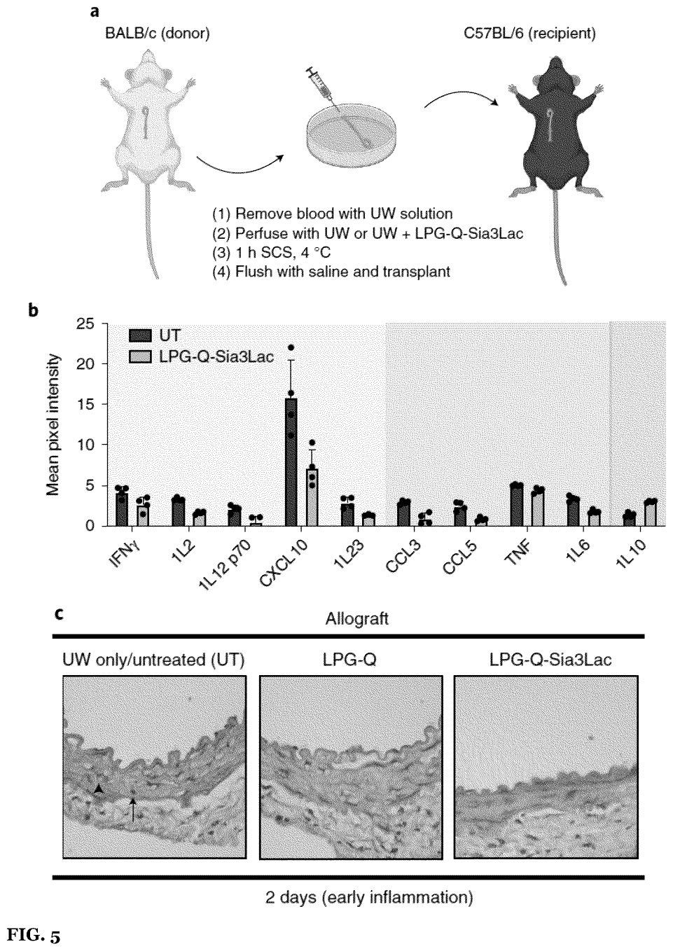

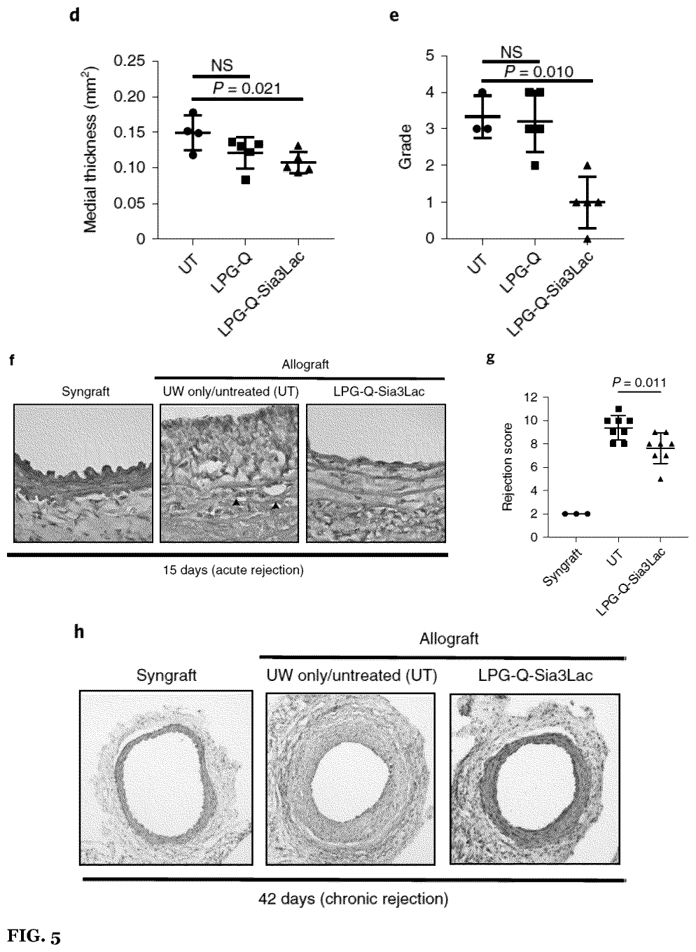

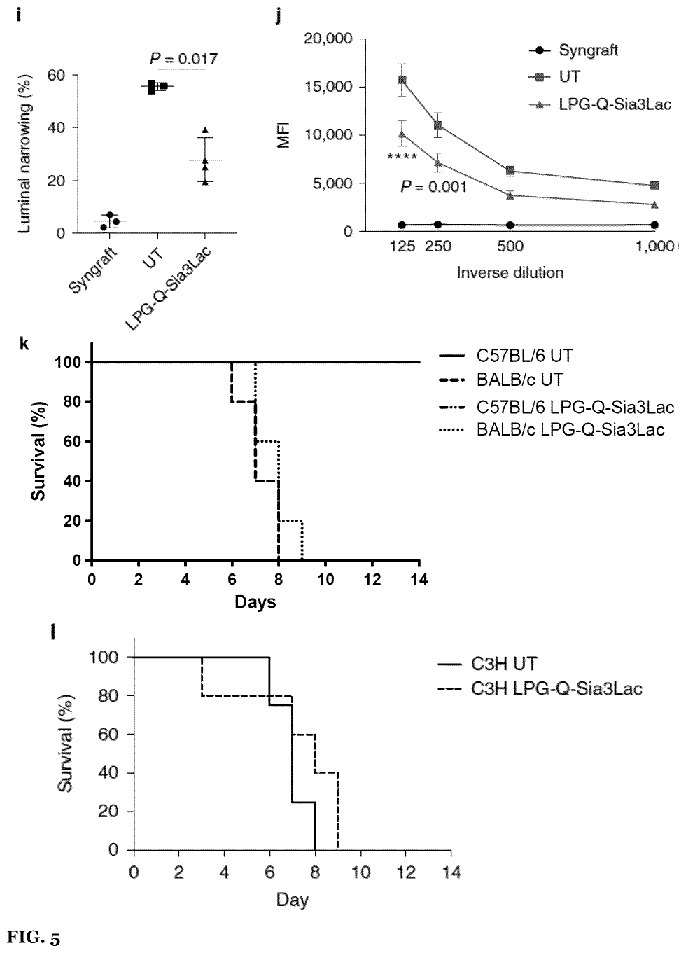

Since CSE of endothelial cells with LPG-Q-Sia3Lac resulted in potent anti-inflammatory effects and immunosuppression in vitro, we examined the therapeutic effect of applying this CSE approach to the prevention of immune-mediated rejection of vascular allografts. A murine aortic allograft transplant model was employed to probe early graft inflammation and acute and chronic rejection (FIG. 5a). In this model, early inflammation of allograft artery segments leads to activation of adaptive immune responses that cause medial injury, a consequence of acute rejection, and intimal thickening, a feature of chronic rejection.^{43,44} Considering the aggressiveness of this immune injury, the aortic interposition model is a useful and stringent model to study therapies that inhibit vascular rejection. Also, rejection in this model involves all aspects of immune responses that contribute to clinical rejection, including T cell-mediated injury as well as de novo development of donor specific antibodies. Finally, vascular rejection is recognized as a central component of heart transplant rejection but is applicable to understanding all organ transplants so these studies are broadly applicable. A segment of the abdominal aorta from BALB/c donor mice was excised, flushed with UW solution, and then bathed in UW solution alone (UT) or UW solution fortified with polymer (LPG-Q, LPG-Q-Sia3Lac) and gtTGase enzyme for 1 hour on ice (FIG. 5a). The excised segments were then flushed with saline and transplanted into the infra-renal aorta of allogenic C57BL/6 recipient mice. The transplanted tissue was removed at day 2 post-transplantation to examine early inflammation, at day 15 to observe acute rejection, and at day 42 to examine chronic rejection.

Swelling of the arterial media and leukocyte infiltration into the vessel wall are features of arterial inflammation. As such, early graft inflammation was first quantified by measuring the thickness of the media at day 2 post-transplantation. Remarkably, LPG-Q-Sia3Lac significantly reduced medial thickness in allograft arteries (FIGS. 5c-e). In addition, medial inflammation was semi-quantitatively graded on a 0-4 scale in a blinded manner. LPG-Q-Sia3Lac reduced histological features of medial inflammation such as medial injury and leukocyte infiltration (FIGS. 5c & e), indicating that LPG-Q-Sia3Lac prevented early inflammation of vascular allografts. This therapeutic effect was not demonstrated on the LPG-Q control, indicating that steric shielding alone with LPG-Q is not sufficient to elicit a therapeutic response in vivo and that immunosuppressive effects of LPG-Q-Sia3Lac are needed along with the steric barrier offered by the polymer. The effect of LPG-Q-Sia3Lac on acute rejection was then assessed in grafts at day 15 post-transplantation. In control untreated grafts, there was fulminant infiltration of the media by leukocytes that was concomitant with extensive medial injury characterized by smooth muscle cell loss, fragmentation of the elastic laminae, and fibrin deposition. Modest intimal thickening was also apparent. In comparison, there was significantly less acute rejection in grafts treated with LPG-Q-Sia3Lac with fewer clusters of infiltrating leukocytes, less elastic laminae damage, no fibrin deposition in the media, and less intimal thickening (FIGS. 5f-g). To determine whether CSE of endothelial cells also affects processes that cause late graft loss, chronic rejection was examined by measuring intimal thickening of allograft arteries at day 42 post-transplantation. There was substantial intimal thickening in untreated (UT) allograft arteries that was substantially reduced by LPG-Q-Sia3Lac (FIGS. 5h-i). These findings indicate that CSE of vascular allografts with LPG-Q-Sia3Lac reduces or prevents early graft inflammation and the resultant immune-mediated vascular injury, thereby leading to long-term graft protection and potent. In addition to cellular immune responses that cause arterial injury, the development of donor-specific antibodies (DSAs) also contributes to rejection. LPG-Q-Sia3Lac significantly reduced the de novo development of DSAs following artery transplantation (FIG. 5j). These findings provide evidence that the graft modification with LPG-Q-Sia3Lac generates a state of ‘immune conditioning’ post-transplantation, which protects grafts from immune-mediated injury thereby minimizing, reducing, or preventing graft rejection or antibody mediated rejection.

Finally, to examine whether the immunosuppressive effect of localized treatment of grafts with LPG-Q-Sia3Lac was restricted to grafts or led to systemic immunosuppression or tolerance, skin grafts were performed onto artery graft recipients at day 28 post-transplantation. There was no rejection of syngeneic skin grafts but skin grafts from Balb/c donors (the same donor strain as artery grafts) were rapidly rejected by day 10 after skin transplantation (FIG. 5k; FIG. 34). Also, third party skin grafts from C3H donors were rejected with similar kinetics as Balb/c grafts (FIG. 34). These findings establish that the protective effects of the CSE approach are limited to the treated graft and do not result in systemic immunosuppression or tolerance, and the mechanism of protection is localized.

All together, these results suggest that the modification of vascular endothelium with LPG-Q-Sia3Lac leads to local immune inactivation. These results emphasize that evading immune recognition and inflammation in the early stages of transplantation can suppress the magnitude of acute and chronic rejection, potentially without the need for systemic immune suppressive drugs.

### Example 4: Protection of Syngeneic and Allogenic Kidney Transplants in Mice

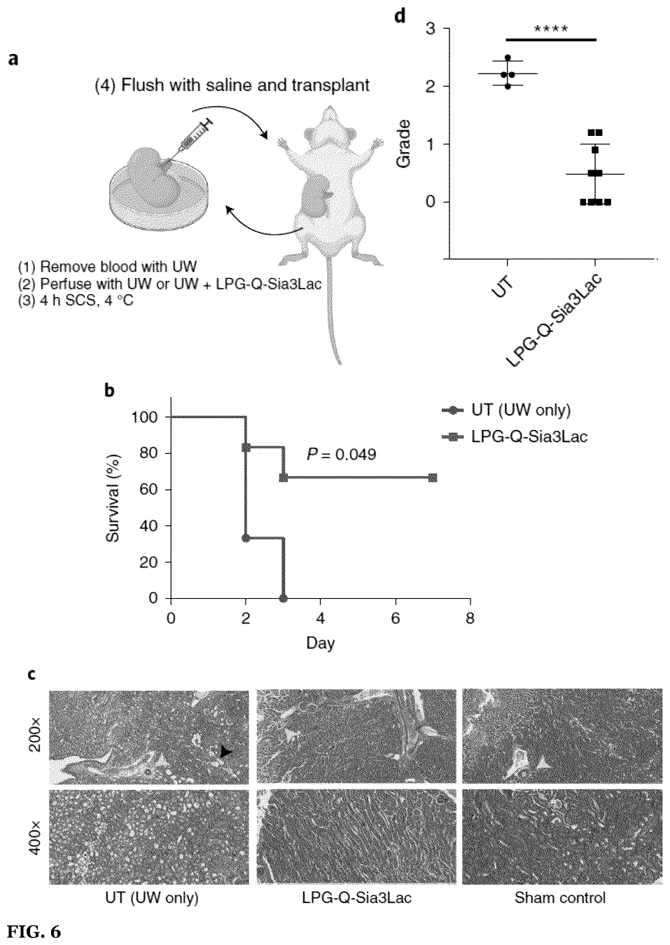

We further tested the ability of our CSE approach to reduce IRI-mediated DGF. To isolate the incidence of DGF from immune-mediated rejection, we used a syngeneic model that subjected kidney transplants to 4 hour cold ischemia storage (CIS). Kidneys were harvested from C57BL/6 mice, flushed with UW solution, and then incubated in UW solution alone (UT) or UW solution fortified with LPG-Q-Sia3Lac and enzyme for 4 hours on ice (FIG. 6a). Following cold storage, the kidney was flushed with saline, transplanted into a syngeneic bi-nephrectomised mouse and observed for 7 days post-transplantation. We found that treatment with UW only (UT) caused significant adverse events that led to mortality for all mice that received UT kidneys within 3 days following the surgical procedure (FIG. 6b), likely due to severe renal function failure. This effect was confirmed by the incidence of histologic damage to the renal tissue as characterized by severe cellular infiltration (blue nuclear staining), tubular damage and large perivascular injury (FIG. 6c). In comparison, more than two thirds of mice treated with the LPG-Q-Sia3Lac solution survived (sacrificed on day 7) and exhibited renal histology that was similar to the sham control (FIG. 6b-c). Subsequent renal tubular necrosis scores of the histology sections from the UT and LPG-Q-Sia3Lac treatment groups demonstrated that CSE significantly decreased the onset of IRI-associated renal damage (FIG. 6d).

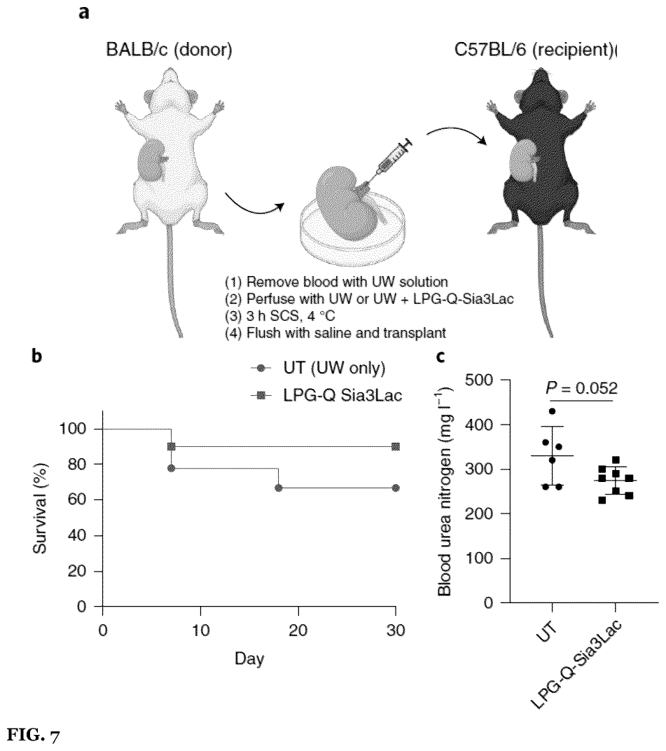

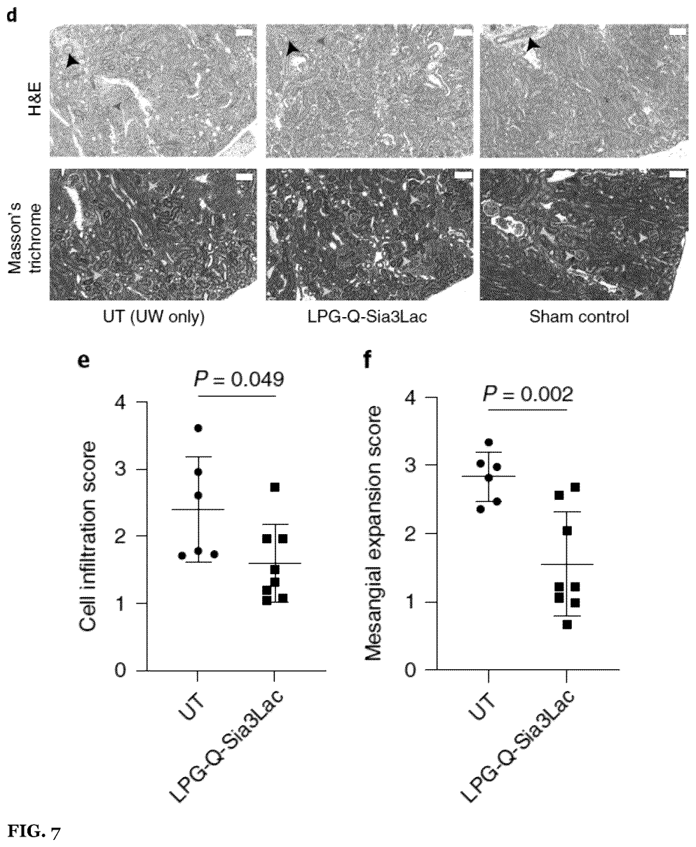

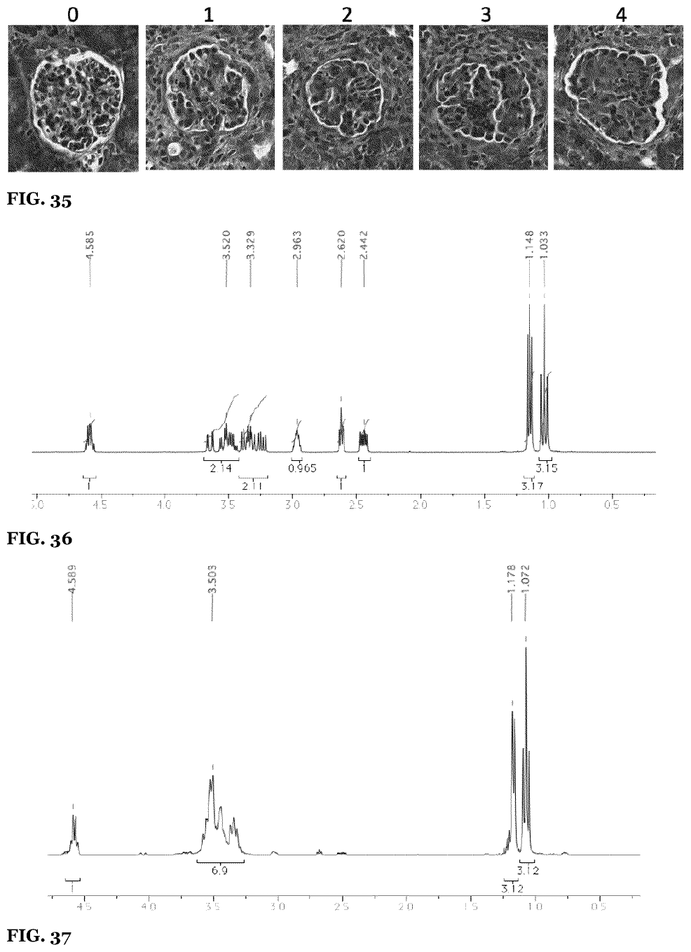

Next, we investigated whether a CSE approach can protect kidney transplants in an allogeneic renal transplantation mouse model. To be consistent with the above-described experiment using aortic transplant model, we chose to use BALB/c as donor and B6 as recipient. BALB/c kidneys are known to be more resistant to IRI than C56BL/6 kidneys, resulting in substantial survival of Balb/c kidney allografts up to day 30 post-transplantation. This provides a longer window to assess the immunological and functional differences between untreated and treated groups.⁵⁰ Kidneys were harvested from BALB/c mice (donor), flushed with UW solution, and then incubated in UW solution alone (UT) or UW solution fortified with LPG-Q-Sia3Lac and enzyme for 3 hours on ice (FIG. 7a). Following CSE, the kidney was flushed with saline, transplanted into an allogeneic bi-nephrectomised B6 mouse (recipient) and observed for 30 days post-transplantation. Treatment of kidney grafts with LPG-Q-Sia3Lac improved survival following transplantation (FIG. 7b), indicated by 7 out of 8 surviving in the LPG-Q-Sia3Lac group as compared to 5 in the UT group. Treatment with LPG-Q-Sia3Lac significantly increased the kidney function in recipient mice as evident from the blood-urea-nitrogen levels (FIG. 7c). Following 30-day transplantation, kidneys were harvested and analyzed for immune cell infiltration and renal glomerular damage histologically (FIG. 7d-f; pictorial reference for mesangial expansion scoring is provided in FIG. 35). FIG. 7d presented a typical microscopic view of cellular infiltration in H&E-stained sections and mesangial expansion in the glomeruli of Masson's trichrome-stained sections in each group. Semi-quantitative histological analysis indicated that both cellular infiltration and glomerular mesangial expansion in renal allografts of the UT group were more severe than those of the LPG-Q-Sia3Lac group (FIG. 7e-f). Taken together, overall CSE with LPG-Q-Sia3Lac significantly protected the kidneys from transplant rejection in an allogenic mouse model.

### Example 5: Schematic of Synthesis of α-Amino Linear Polyglycerol

To achieve linear polyglycerol, the pendant hydroxyl group was modified using ethyl vinyl ether to yield ethoxyethyl glycidyl ether as the monomer. Then using tetrabutylammonium azide as the initiator for a ring opening polymerization reaction, then a polymer with an azide group in the alpha position was yielded. Finally, through a series of deprotection reactions and a reduction reaction using triphenyl phosphine allowed for the final polymer, α-amino linear polyglycerol.

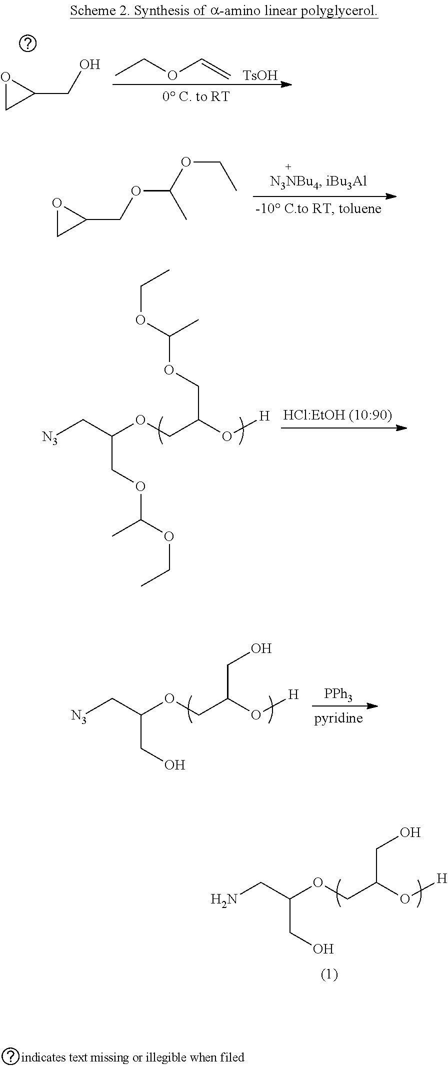

### Example 6: Schematic of Synthesis of Polymer Peptide Conjugation

To allow for cell surface engineering, the peptide was attached through an amide bond formation using the amine group of the polymer and the carboxylic acid group from the peptide. The glutamines in the peptide are trityl protected to ensure specificity. Sulfated versions of this polymer were sulfated during the same reaction. Ultimately, the polymers were acid deprotected to yield linear polyglycerol sulfate glutamine.

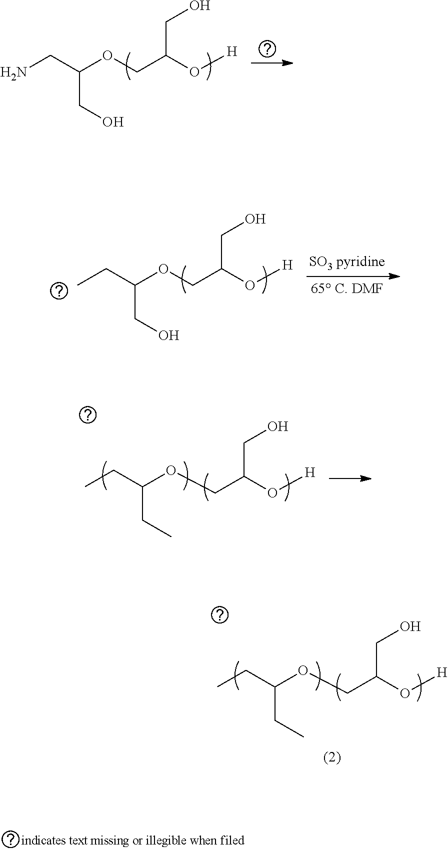

Scheme 3. Synthesis of polymer peptide conjugation using linear polyglycerol sulfate and glutamine-containing tissue transglutaminase recognition peptide.

### Example 7: Chromatographic and Spectroscopic Characterizations

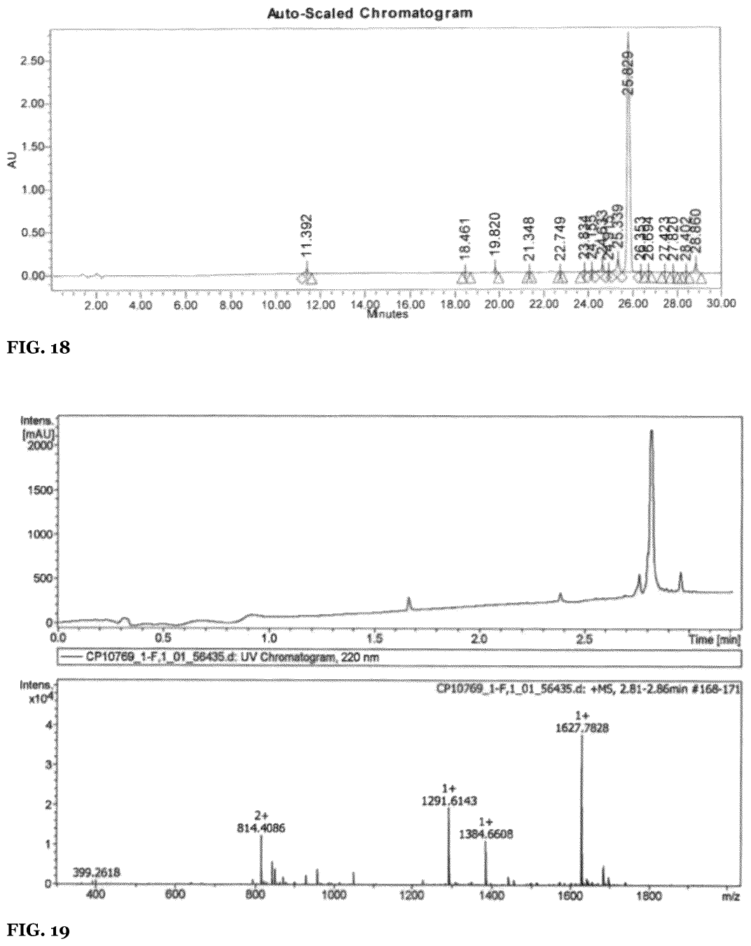

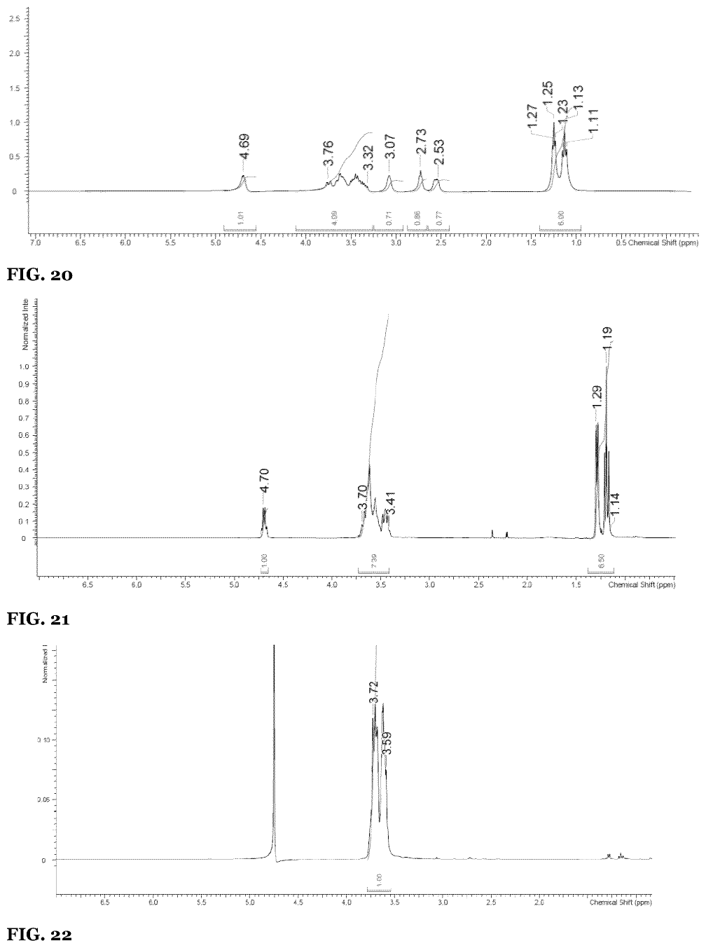

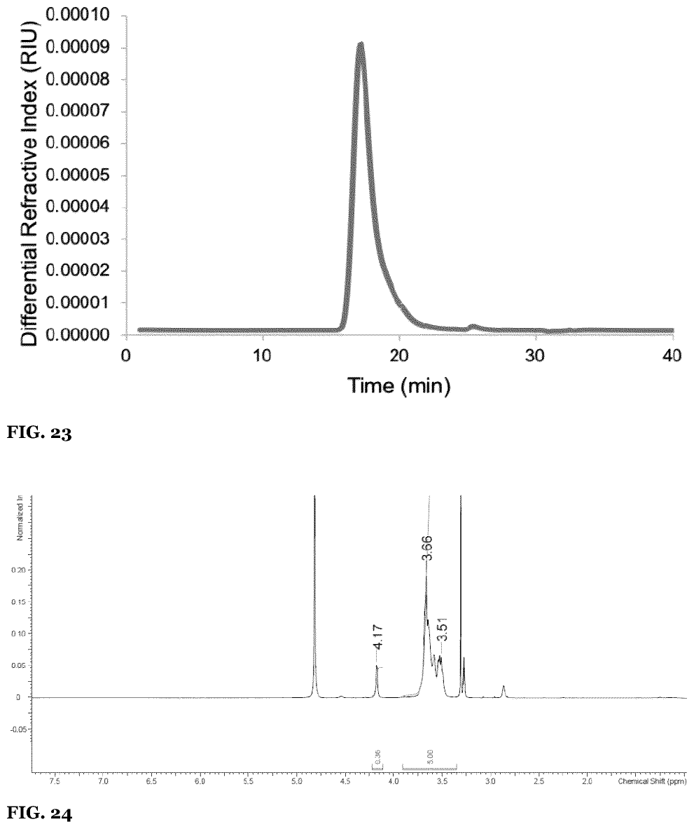

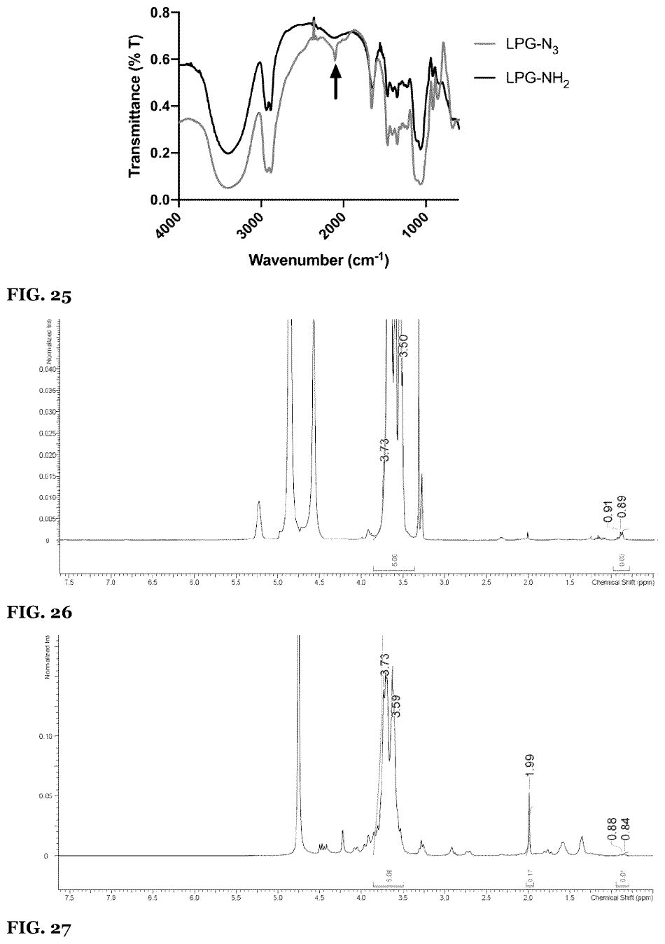

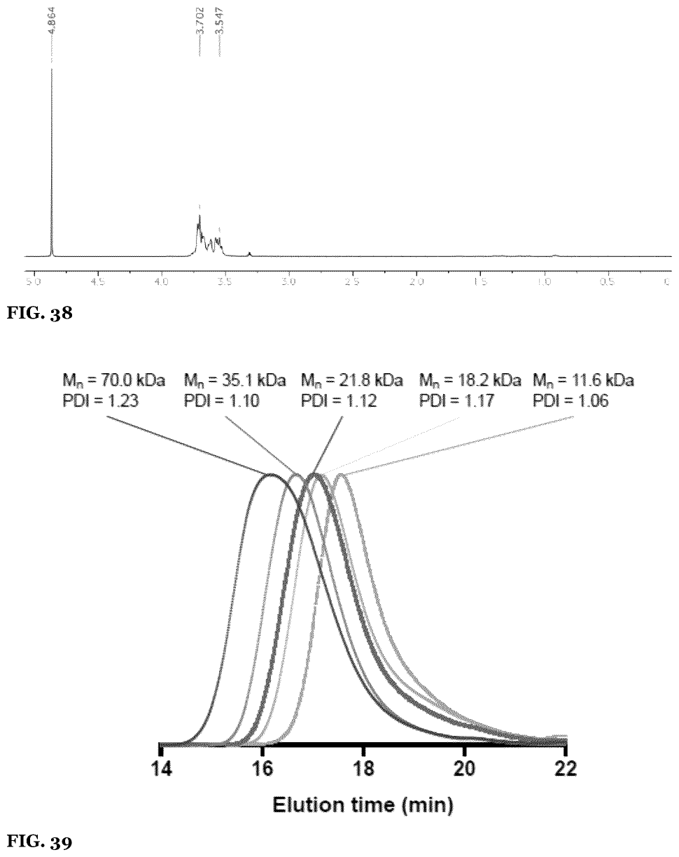

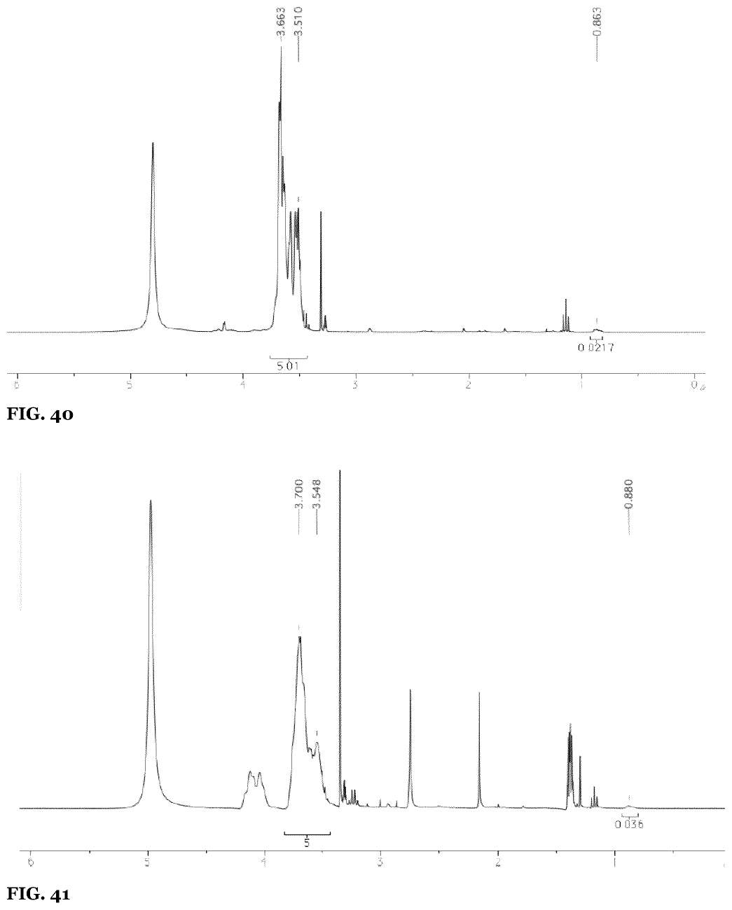

A description of chromatographic and spectroscopic characterization information (FIGS. 18-30) for the purity and synthesis of LPG, LPG-Q, LPG-Q-Sia3Lac, LPG-Q-Lac, LPG-Q-Sia6Lac provided (see Scheme 1), demonstrating the final structures of the intermediates of LPG-Q-Sia3Lac, LPG-Q-Sia6Lac and their final forms (FIGS. 18-30). FIGS. 36-41 are chromatographic and spectroscopic information for the purity and synthesis of LPG, LPG-Q, and LPG-Q.

### Example 8: Engineering Endothelial Cells with Anti-Oxidant and Anti-Inflammatory Polymers Scavenges Toxic Reactive Oxygen Species and Reduces Inflammatory Injury

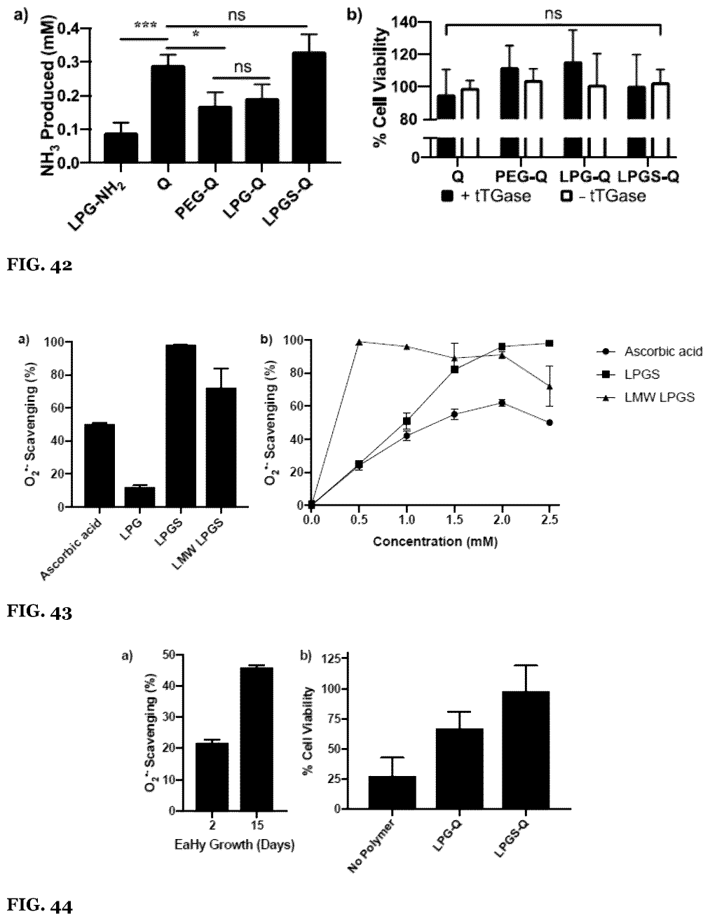

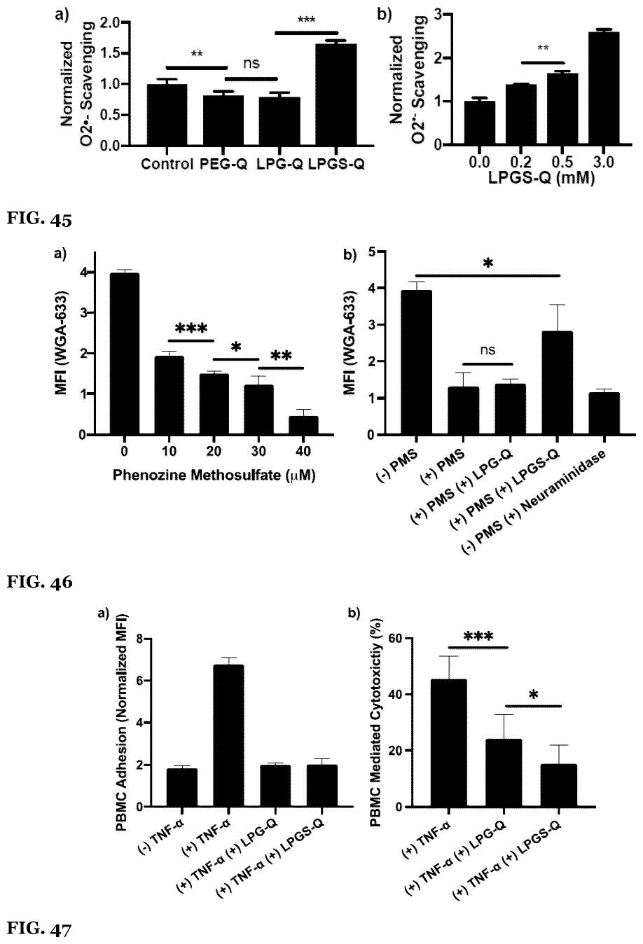

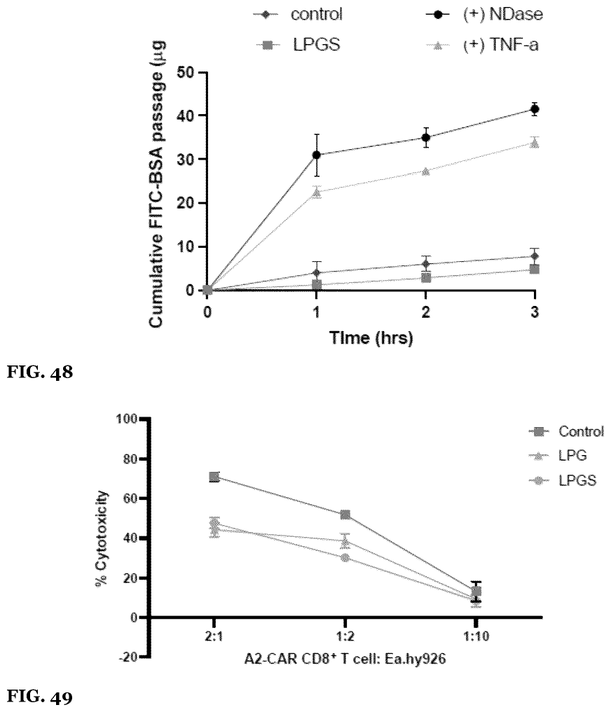

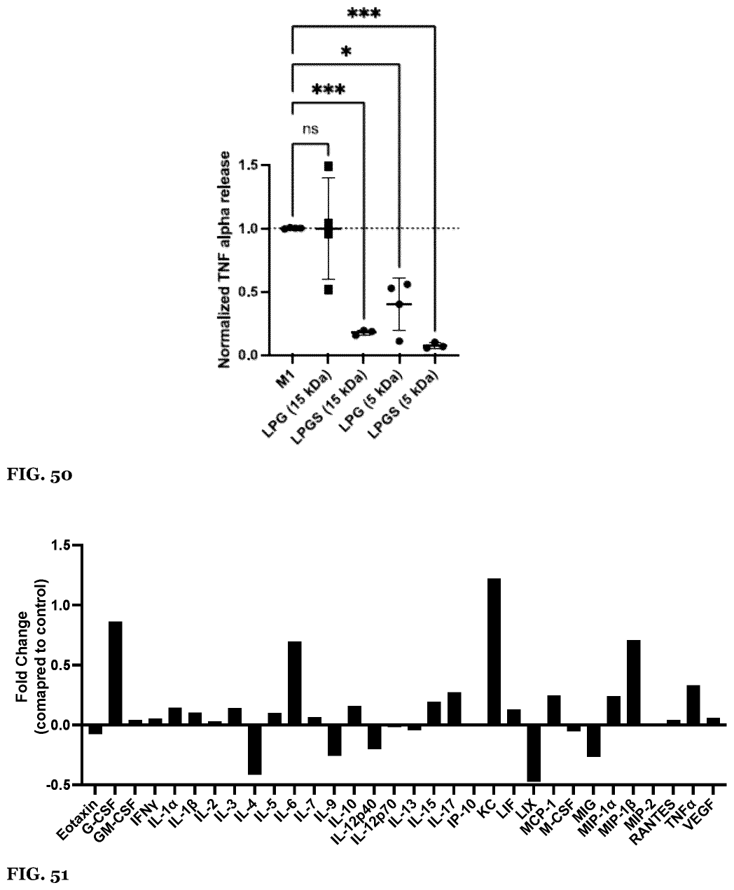

Using gtTGase, sulfated linear polyglycerols (LPGS) having a Q-tag (LPGS-Q) were tested for their biocompatibility on endothelial cell surfaces (EA.hy926 cells). Production of NH₃ is a biproduct of transglutamination and can be used to assess the efficiency of surface ligation of the enzyme. LPGS-Q showed higher ammonia production than its non-sulfated counterparts suggesting more reactivity on cell surfaces without affecting cell metabolic activity (FIG. 42) demonstrating great cell compatibility. Reactive oxygen species (ROS) are harmful to cells and can lead to oxidative damage. By assessing the ability of LPGS to scavenge superoxide anions we can evaluate its anti-oxidant properties. LPGS is able to scavenge superoxide radicals in solution in a concentration manner (FIG. 43) and its anti-oxidant effects are retained when immobilized on the cell surface through CSE using gtTGase (FIG. 45). Under various growth conditions cells have varying abilities to scavenge superoxide radicals to reduce cellular damage, however, after 15 days, though scavenge activity is higher, cell viability is drastically impaired; pairing LPGS-Q with CSE using gtTGase allows for the rescue of the cells viability by scavenging the superoxide radicals (FIG. 44). One mechanism by which cells are damaged by oxidative stress is through the shedding of the glycocalyx structure. Through the same experiments, we were able to specifically probe for the glycocalyx structure using flow cytometry and quantify its abundance after exposure to superoxide anions; LPGS-Q-modified cells showed a significantly larger retention of the glycocalyx structure compared to non-sulfated counterparts (FIG. 46). Moreover, transendothelial passage of a monolayer is also directly connected to its glycocalyx structure retention as the more damaged the glycocalyx, the leaker the membrane will be and non-specific macromolecule passage will occur. Similarly, LPS-Q-modified cells were able to reduce non-specific macromolecule passage (FIG. 48). Besides oxidative damage, endothelial cells can sustain injury from immune cells directly through recognition or indirectly through pro-inflammatory responses. LPGS-Q modified endothelial cells, when incubated with PBMCs, possessed immunoevasive properties (by evading recognition and subsequent adhesion) and immunosuppressive properties (by reducing PBMC-mediated cytotoxicity; FIG. 47). Under more specific conditions using HLA-A2-reactive T cells, LPGS-Q polymers performed similarly to non-sulfated counterparts in reducing cytotoxicity imparted by the HLA-A2-reactive T cells (FIG. 49); suggesting a non-specific level of immunosuppression when directly interacting with immune cells. Finally, macrophages that exacerbate inflammation (M1 state) are harmful as they limit anti-inflammatory activity which can lead to halted healing of cells. In the presence of sulfated linear polyglycerols (LPGS) macrophages in M1 state inhibited secretion of TNF-α, a pro-inflammatory cytokine (FIG. 50). Taken together, LPGS is able to suppress the activity of M1-macrophages, thereby inhibiting a part of the pro-inflammatory cascade leading to reduced inflammatory activity.

### Example 9: Cell Surface Engineering with LPGS-Q for Aortic Transplants

Sulfonated linear polyglycerols (LPGS) having a Q-tag (LPGS-Q) were tested in allogeneic aortic interposition grafts with from BALB/c donor mice for transplant into C57/BL/6 recipient mice. All polymer ligations to the aortic graft were performed in UW solution fortified with 0.5 mM LPGS-Q, 3 mM GSH, 5 mM CaCl₂ and 0.2 U/mL gtTGase for 1 h at 4° C., while the untreated control groups were treated with UW solution fortified with 3 mM GSH, 5 mM CaCl₂ and 0.2 U/mL gtTGase. Cytokine profile of the sera of recipient mice 2 days post-transplant (FIG. 51) and 15 days post-transplant (FIG. 52). These results show that modifying the artery locally with LPGS-Q showed beneficial anti-inflammatory properties post-transplantation.

### Example 10: Cell Surface Engineering with LPGS-Q for Cell Transplants

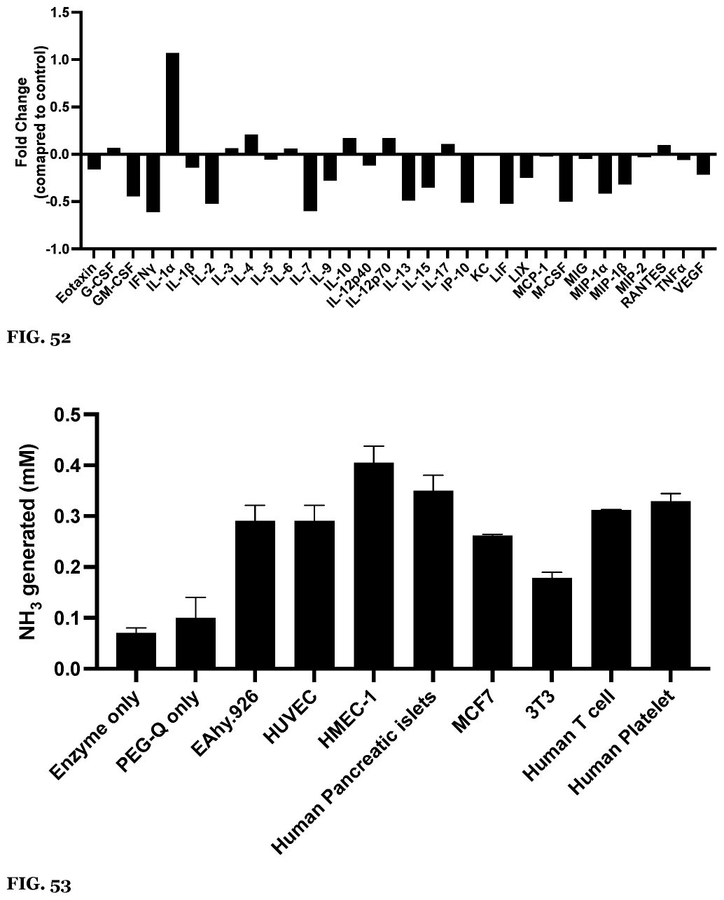

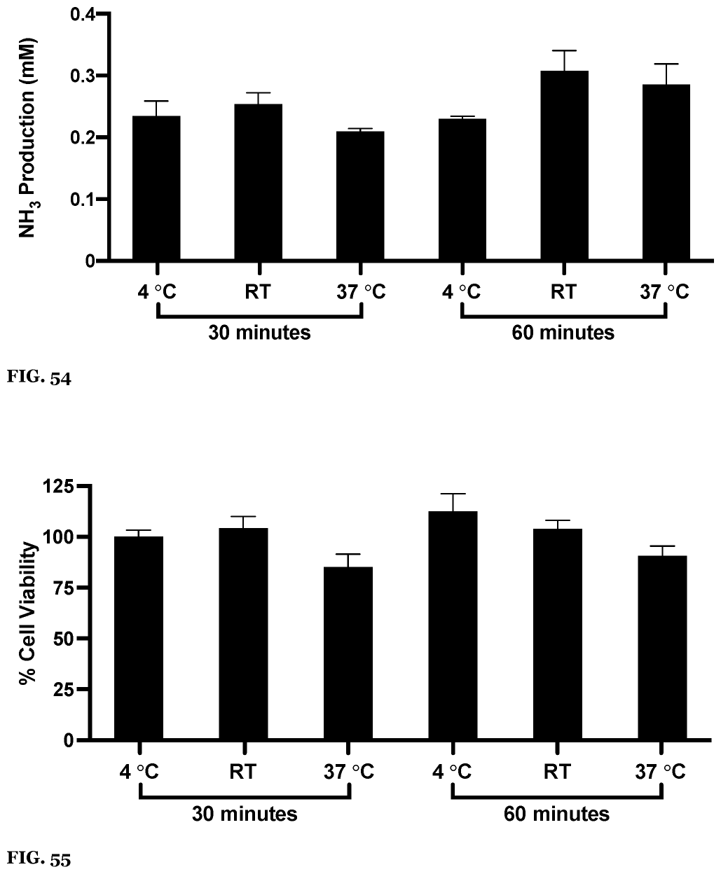

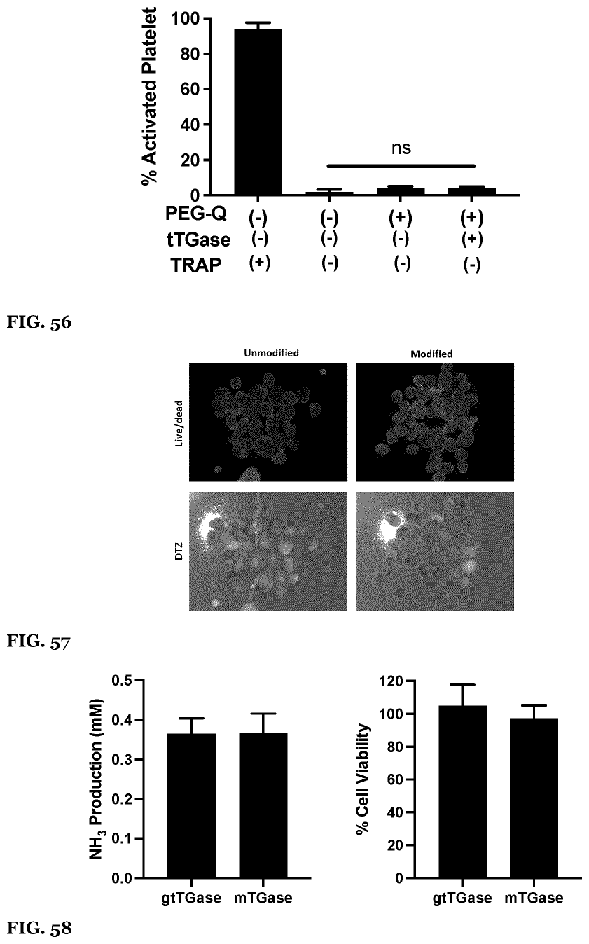

Various cells were incubated with Q-tagged polymer (LPGS-Q) and conjugation efficiency was assessed by NH₃ production, which is a bi-product in the transglutamination reaction when using gtTGase (see FIG. 53). Similarly, EA.hy926 cells were surface engineered (endothelial glycocalyx) using gtTGase and LPGS-Q at various temperatures and time intervals, NH₃ production is shown in FIG. 54 and percent cell viability is shown in FIG. 55. Platelets were similarly surface modified with LPGS-Q, which did not induce platelet activation as assessed by flow cytometry using CD62 surface markers, with TRAP used as a positive control for platelet activation (see FIG. 56).

Furthermore, the compatibility of cell surface engineering on human pancreatic islet surfaces with LPGS-Q using gtTGase, was tested for cell viability (i.e. assessed using a LIVE/DEAD stain assay and insulin producing activity was assessed by DTZ detection assay, as compared to unmodified cells). We were able to demonstrate that Q-tagged polymers (i.e. LPGS-Q) can be attached to different cell surfaces using various reaction conditions and still produce viable cells (see FIG. 57). Cell surface engineering of endothelial glycocalyx using TGase from guinea pig liver (gtTGase) and microbial origin (mTGase) isoforms were compared for both NH₃ production and cell viability, with favourable results for both gtTGase and mTGase on two different protein tags (i.e. GQQQLGGGGG-PEG and WLAQRPH-PEG).

The disclosure may be further understood by the non-limiting examples. Although the description herein contains many specific examples, these should not be construed as limiting the scope of the disclosure but as merely providing illustrations of some of the embodiments of the disclosure. For example, thus the scope of the disclosure should be determined by the appended aspects and their equivalents, rather than by the examples given.

Many of the molecules disclosed herein contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this disclosure for a given application. In specific applications, the selection of a given anion or cation for preparation of a salt may result in increased or decreased solubility of that salt. Every formulation or combination of components described or exemplified herein may be used to practice the disclosure, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the aspects herein.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to an embodiment of the present invention. The invention includes all embodiments and variations substantially as hereinbefore described and with reference to the examples and drawings. Unless otherwise explained, all technical and scientific terms used herein have the same meaning as commonly understood by one of ordinary skill in the art to which a disclosed disclosure belongs.

### REFERENCES

1. Chambers, D. C. et al. The International Thoracic Organ Transplant Registry of the International Society for Heart and Lung Transplantation: thirty-fifth adult lung and heart-lung transplant report-2018; focus theme: multiorgan transplantation. J. Heart Lung Transplant. 37, 1169-1183 (2018).
2. Moini, M., Schilsky, M. L. & Tichy, E. M. Review on immunosuppression in liver transplantation. World J. Hepatol. 7, 1355-1368 (2015).
3. Shivaswamy, V., Boerner, B. & Larsen, J. Post-transplant diabetes mellitus: causes, treatment, and impact on outcomes. Endocr. Rev. 37, 37-61 (2016).
4. Gutierrez-Dalmau, A. & Campistol, J. M. Immunosuppressive therapy and malignancy in organ transplant recipients: a systematic review. Drugs 67, 1167-1198 (2007).
5. Meier-Kriesche, H. U. et al. Immunosuppression: evolution in practice and trends, 1994-2004. Am. J. Transplant. 6, 1111-1131 (2006).
6. Kalluri, H. V. & Hardinger, K. L. Current state of renal transplant immunosuppression: present and future. World J. Transplant. 2, 51-68 (2012).
7. Gammon, J. M. & Jewell, C. M. Engineering immune tolerance with biomaterials. Adv. Healthc. Mater. 8, 1801419 (2019).
8. Uehara, M. et al. Nanodelivery of mycophenolate mofetil to the organ improves transplant vasculopathy. ACS Nano 13, 12393-12407 (2019).
9. Dane, K. Y. et al. Nano-sized drug-loaded micelles deliver payload to lymph node immune cells and prolong allograft survival. J. Control. Release 156, 154-160 (2011).
10. Azzi, J. et al. Targeted delivery of immunomodulators to lymph nodes. Cell Rep. 15, 1202-1213 (2016).
11. Wu, J. et al. Immune responsive release of tacrolimus to overcome organ transplant rejection. Adv. Mater. 30, 1805018 (2018).
12. Solhjou, Z. et al. Novel application of localized nanodelivery of anti-interleukin-6 protects organ transplant from ischemia-reperfusion injuries. Am. J. Transplant. 17, 2326-2337 (2017).
13. Lamprecht, A., Yamamoto, H., Takeuchi, H. & Kawashima, Y. A pH-sensitive microsphere system for the colon delivery of tacrolimus containing nanoparticles. J. Control. Release 104, 337-346 (2005).
14. Valenzuela, N. M. & Reed, E. F. Antibody-mediated rejection across solid organ transplants: manifestations, mechanisms, and therapies. J. Clin. Invest. 127, 2492-2504 (2017).
15. Salvadori, M., Rosso, G. & Bertoni, E. Update on ischemia-reperfusion injury in kidney transplantation: pathogenesis and treatment. World J. Transplant. 5, 52-67 (2015).
16. Incerti, D. et al. The lifetime health burden of delayed graft function in kidney transplant recipients in the United States. MDM Policy Pract. 3, 2381468318781811 (2018).
17. Benzimra, M., Calligaro, G. L. & Glanville, A. R. Acute rejection. J. Thorac. Dis. 9, 5440-5457 (2017).
18. Naito, H. et al. Therapeutic strategies for ischemia reperfusion injury in emergency medicine. Acute Med. Surg. 7, e501 (2020).
19. Charlton, M. et al. International liver transplantation society consensus statement on immunosuppression in liver transplant recipients. Transplantation 102, 727-743 (2018).
20. Sakai, K. et al. Protective effect and mechanism of IL-10 on renal ischemia-reperfusion injury. Lab. Investig. 99, 671-683 (2019).
21. Sawitzki, B. et al. Regulatory cell therapy in kidney transplantation (The ONE Study): a harmonised design and analysis of seven non-randomised, single-arm, phase 1/2A trials. Lancet 395, 1627-1639 (2020).
22. Harden, P. N. et al. Feasibility, long-term safety, and immune monitoring of regulatory T cell therapy in living donor kidney transplant recipients. Am. J. Transplant. 21, 1603-1611 (2021).
23. Peng, B., Ming, Y. & Yang, C. Regulatory B cells: the cutting edge of immune tolerance in kidney transplantation. Cell Death Dis. 9, 109 (2018).
24. Sasaki, H. et al. Preclinical and clinical studies for transplant tolerance via the mixed chimerism approach. Hum. Immunol. 79, 258-265 (2018).
25. Lowsky, R. & Strober, S. Combined kidney and hematopoeitic cell transplantation to induce mixed chimerism and tolerance. Bone Marrow Transplant. 54, 793-797 (2019).
26. Lee, K. W. et al. Inducing transient mixed chimerism for allograft survival without maintenance immunosuppression with combined kidney and bone marrow transplantation: protocol optimization. Transplantation 104, 1472-1482 (2020).
27. Brasile, L., Glowacki, P., Castracane, J. & Stubenitsky, B. M. Pretransplant kidney-specific treatment to eliminate the need for systemic immunosuppression. Transplantation 90, 1294-1298 (2010).
28. Bellini, M. I. & D'Andrea, V. Organ preservation: which temperature for which organ?J. Int. Med. Res. 47, 2323-2325 (2019).
29. Hosgood, S. A., Hoff, M. & Nicholson, M. L. Treatment of transplant kidneys during machine perfusion. Transpl. Int. 34, 224-232 (2021).
30. Rehm, M. et al. Shedding of the endothelial glycocalyx in patients undergoing major vascular surgery with global and regional ischemia. Circulation 116, 1896-1906 (2007).
31. Abrahimi, P., Liu, R. & Pober, J. S. Blood vessels in allotransplantation. Am. J. Transplant. 15, 1748-1754 (2015).
32. Piotti, G., Palmisano, A., Maggiore, U. & Buzio, C. Vascular endothelium as a target of immune response in renal transplant rejection. Front. Immunol. 5, 1-9 (2014).
33. Kollar, B. et al. The significance of vascular alterations in acute and chronic rejection for vascularized composite allotransplantation. J. Vasc. Res. 56, 163-180 (2019).
34. Rao, D. A. & Pober, J. S. Endothelial injury, alarmins, and allograft rejection. Crit. Rev. Immunol. 28, 229-248 (2008).
35. Colvin, R. B. & Smith, R. N. Antibody-mediated organ-allograft rejection. Nat. Rev. Immunol. 5, 807-817 (2005).
36. Cross, A. R., Glotz, D. & Mooney, N. The role of the endothelium during antibody-mediated rejection: from victim to accomplice. Front. Immunol. 9, 106 (2018).
37. Lin, C. W. & Ting, A. Y. Transglutaminase-catalyzed site-specific conjugation of small-molecule probes to proteins in vitro and on the surface of living cells. J. Am. Chem. Soc. 128, 4542-4543 (2006).
38. Abbina, S., Siren, E. M. J., Moon, H. & Kizhakkedathu, J. N. Surface engineering for cell-based therapies: techniques for manipulating mammalian cell surfaces. ACS Biomater. Sci. Eng. 4, 3658-3677 (2018).
39. Imran Ul-Haq, M., Lai, B. F. L., Chapanian, R. & Kizhakkedathu, J. N. Influence of architecture of high molecular weight linear and branched polyglycerols on their biocompatibility and biodistribution. Biomaterials 33, 9135-9147 (2012).
40. MacAuley, M. S., Crocker, P. R. & Paulson, J. C. Siglec-mediated regulation of immune cell function in disease. Nat. Rev. Immunol. 14, 653-666 (2014).
41. Duan, S. & Paulson, J. C. Siglecs as immune cell checkpoints in disease. Annu. Rev. Immunol. 38, 365-395 (2020).
42. Ramnath, R. et al. Matrix metalloproteinase 9-mediated shedding of syndecan 4 in response to tumor necrosis factor α: a contributor to endothelial cell glycocalyx dysfunction. FASEB J. 28, 4686-4699 (2014).
43. Aronson, F. R., Libby, P., Brandon, E. P., Janicka, M. W. & Mier, J. W. IL-2 rapidly induces natural killer cell adhesion to human endothelial cells. a potential mechanism for endothelial injury. J. Immunol. 141, 158-163 (1988).
44. Spence, S. et al. Targeting Siglecs with a sialic acid-decorated nanoparticle abrogates inflammation. Sci. Transl. Med. 7, 1-13 (2015).
45. Zaccai, N. R. et al. Structure-guided design of sialic acid-based Siglec inhibitors and crystallographic analysis in complex with sialoadhesin. Structure 11, 557-567 (2003).
46. Wu, J., Xie, A. & Chen, W. Cytokine regulation of immune tolerance. Burns Trauma 2, 2321-3868.124771 (2014).
47. Dawson, N. A. J. et al. Functional effects of chimeric antigen receptor co-receptor signaling domains in human regulatory T cells. Sci. Transl. Med. 12, 3866 (2020).
48. Enns, W., von Rossum, A. & Choy, J. Mouse model of alloimmune-induced vascular rejection and transplant arteriosclerosis. J. Vis. Exp. 2015, 1-7 (2015).
49. Choy, J. C. et al. Granzyme B induces endothelial cell apoptosis and contributes to the development of transplant vascular disease. Am. J. Transplant. 5, 494-499 (2005).
50. von Rossum, A. et al. Bim regulates alloimmune-mediated vascular injury through effects on T-cell activation and death. Arterioscler. Thromb. Vasc. Biol. 34, 1290-1297 (2014).
51. Von Rossum, A. et al. Graft-derived IL-6 amplifies proliferation and survival of effector T cells that drive alloimmune-mediated vascular rejection. Transplantation 100, 2332-2341 (2016).
52. Duong Van Huyen, J. et al. The XVth Banff conference on allograft pathology the Banff workshop heart report: improving the diagnostic yield from endomyocardial biopsies and Quilty effect revisited. Am. J. Transplant. 20, 3308-3318 (2020).
53. Hudak, J. E., Canham, S. M. & Bertozzi, C. R. Glycocalyx engineering reveals a Siglec-based mechanism for NK cell immunoevasion. Nat. Chem. Biol. 10, 69-75 (2013).
54. Shoskes, D. A. & Cecka, J. M. Deleterious effects of delayed graft function in cadaveric renal transplant recipients independent of acute rejection. Transplantation 66, 1697-1701 (1998).
55. Qiu, L. et al. Kidney-intrinsic factors determine the severity of ischemia/reperfusion injury in a mouse model of delayed graft function. Kidney Int. 98, 1489-1501 (2020).
56. Rink, J. S. et al. Nitric oxide-delivering high-density lipoprotein-like nanoparticles as a biomimetic nanotherapy for vascular diseases. ACS Appl. Mater. Interfaces 10, 6904-6916 (2018).
57. Rogers, N. M., Zhang, Z. J., Wang, J. J., Thomson, A. W. & Isenberg, J. S. CD47 regulates renal tubular epithelial cell self-renewal and proliferation following renal ischemia reperfusion. Kidney Int. 90, 334-347 (2016).
58. Cheng, C. W. et al. Calcium-binding proteins annexin A2 and S100A6 are sensors of tubular injury and recovery in acute renal failure. Kidney Int. 68, 2694-2703 (2005).
59. Giwa, S. et al. The promise of organ and tissue preservation to transform medicine. Nat. Biotechnol. 35, 530-542 (2017).
60. Eshmuminov, D. et al. An integrated perfusion machine preserves injured human livers for 1 week. Nat. Biotechnol. 38, 189-198 (2020).
61. Weinbaum, S., Zhang, X., Han, Y., Vink, H. & Cowin, S. C. Mechanotransduction and flow across the endothelial glycocalyx. Proc. Natl Acad. Sci. USA 100, 7988-7995 (2003).
62. Karlhofer, F. M., Ribaudo, R. K. & Yokoyama, W. M. MHC class I alloantigen specificity of Ly-49+IL-2-activated natural killer cells. Nature 358, 66-70 (1992).
63. Murphy, W. J., Kumar, V. & Bennett, M. Rejection of bone marrow allografts by mice with severe combined immune deficiency (SCID). evidence that natural killer cells can mediate the specificity of marrow graft rejection. J. Exp. Med. 165, 1212-1217 (1987).
64. Zhang, Z.-X. et al. NK cells induce apoptosis in tubular epithelial cells and contribute to renal ischemia-reperfusion injury. J. Immunol. 181, 7489-7498 (2008).
65. Choy, J. C., Kerjner, A., Wong, B. W., McManus, B. M. & Granville, D. J. Perforin mediates endothelial cell death and resultant transplant vascular disease in cardiac allografts. Am. J. Pathol. 165, 127-133 (2004).
66. Chen, G.-Y. et al. Broad and direct interaction between TLR and Siglec families of pattern recognition receptors and its regulation by Neu1. eLife 3, e04066 (2014).
67. Perdicchio, M. et al. Sialic acid-modified antigens impose tolerance via inhibition of T-cell proliferation and de novo induction of regulatory T cells. Proc. Natl Acad. Sci. USA 113, 3329-3334 (2016).
68. Toubai, T. et al. Siglec-G represses DAMP-mediated effects on T cells. JCI Insight 2, e92293 (2017).
69. Wu, Y., Ren, D. & Chen, G.-Y. Siglec-E negatively regulates the activation of TLR4 by controlling its endocytosis. J. Immunol. 197, 3336-3347 (2016).
70. Uehara, S. et al. NK cells can trigger allograft vasculopathy: the role of hybrid resistance in solid organ allografts. J. Immunol. 175, 3424-3430 (2005).
71. McNerney, M. E. et al. Role of natural killer cell subsets in cardiac allograft rejection. Am. J. Transplant. 6, 505-513 (2006).
72. Friend, P. J. Strategies in organ preservation—a new golden age. Transplantation 104, 1753-1755 (2020).
73. Van Nieuw Amerongen, G. P. & Van Hinsbergh, V. W. M. in Adhesion Protein Protocols. Methods in Molecular Biology Vol. 96 (eds Dejana, E. & Corada, M.) 183-189 (Humana Press, 1999).
74. Rey, K. et al. Disruption of the gut microbiota with antibiotics exacerbates acute vascular rejection. Transplantation 102, 1085-1095 (2018).
75. Cheng, C. H. et al. Murine full-thickness skin transplantation. J. Vis. Exp. 2017, 55105 (2017).
76. Pakyari, M. et al. A new method for skin grafting in murine model. Wound Repair Regen. 24, 695-704 (2016).
77. Zhang, Z. et al. Improved techniques for kidney transplantation in mice. Microsurgery 16, 103-109 (1995).
78. Lerret, N. M. et al. Recipient Myd88 deficiency promotes spontaneous resolution of kidney allograft rejection. J. Am. Soc. Nephrol. 26, 2753-2764 (2015).
79. Nuijens, T. et al. Natural Occurring and Engineered Enzymes for Peptide Ligation and Cyclization Front. Chem., 7, 829 (2019).
80. Huang, M. L. et al. Glycocalyx scaffolding with synthetic nanoscale glycomaterials Biomater Sci. 5(8): 1537-1540 (2017).
81. Weeks, A. M. and Wells, J. A. Subtiligase-Catalyzed Peptide Ligation Chem. Rev. 120, 3127-3160 (2020).
82. Weeks, A. M. and Wells, J. A. Engineering peptide ligase specificity by proteomic identification of ligation sites Nature Chemical Biology 14:50-57 (2018).
83. Taki, M.; Shiota, M.; Taira, K. Transglutaminase-Mediated N- and C-Terminal Fluorescein Labeling of a Protein Can Support the Native Activity of the Modified Protein. Protein Eng. Des. Sel. 17 (2), 119-126 (2004).
84. Jones, M. E. R.; Messersmith, P. B. Facile Coupling of Synthetic Peptides and Peptide-Polymer Conjugates to Cartilage via Transglutaminase Enzyme. Biomaterials 28 (35), 5215-5224 (2007).
85. Gervais, M. et al. Direct Synthesis of α-Azido,ω-hydroxypolyethers by Monomer-Activated Anionic Polymerization Macromolecules 42, 7, 2395-2400 (2009).

1. A compound, the compound having the structure of Formula A: 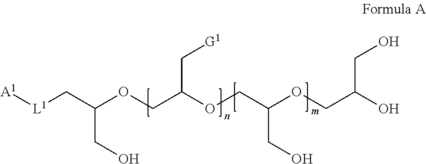
wherein,
A¹ is a peptide tag;
L¹ is a peptide linking group;
G¹ is selected from: -a linker-(a sugar)_{q1}-(a sialic acid)_{q2}; and —R¹—SO₃⁻ group;
n is an integer between 1 and 40,000;
m is an integer between 1 and 40,000;
R¹ is selected from C, O and N;
the linker is selected from: an alkyl chain; a substituted alkyl chain; or an alkyl chain containing an azole ring;
q1 is an integer between 1 and 10; and
q2 is an integer between 1 and 10.

2. The compound of claim 1, wherein the peptide tag is selected from one or more of: GQQQLG; GQQQLGGGG; GQQQLGGGGG; GQQQLGGGGGGGGG; WLAQRPH; PKPQQFM; GQLKHLEQQEG; PNPQLPF; NQEQVSPLTLLK; TVQQEL; QVPL; QQPL; NGL; LPETG; AAPC-glycolate-FG; and KAAPC-glycolate-FG.

3. The compound of claim 1, wherein the peptide tag is a glutamine donor.

4. The compound of claim 3, wherein the glutamine donor is between 3 and 30 amino acids and includes at least one glutamine (Q).

5. The compound of claim 3, wherein the glutamine donor has one of more glycine (G) spacers.

6. (canceled)

7. The compound of claim 1, wherein the peptide tag is selected from: GQQQLG; GQQQLGGGG; GQQQLGGGGG; GQQQLGGGGGGGGG; WLAQRPH; PKPQQFM; GQLKHLEQQEG; PNPQLPF;
NQEQVSPLTLLK; TVQQEL; QVPL; and QQPL.

8. The compound of claim 1, wherein peptide linking group is selected from: an amide group; an alcohol group; an amine group; a thiol group; an azide group; an alkyne group; an alkene group; a carboxylic acid group; an aldehyde group; a ketone group; a halogen group; an isocyanate group; an isothiocyanate group; an oligoethylene glycol group; and a Michael acceptor/donor group.

9. The compound of claim 1, wherein the sugar is selected from: a monosaccharide; a disaccharide; and an oligosaccharide.

10. The compound of claim 1, wherein the sugar is selected from: lactose; N-acetylgalactosamine (GalNac); galactose β(1-3)N-acetyllactosamine (Galβ(1-3)GalNAc); N-acetyllactosamine (LacNAc); Galβ1-4GlcNAc; Gal β1,3GlcNAc; and Gal β 1,3Glc.

11. The compound of claim 1, wherein the linker is selected from: 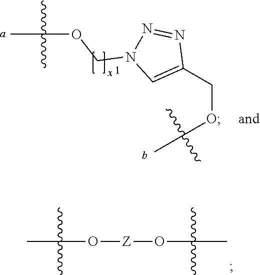
wherein,
position a connects to the sugar and b position connects to the linear polyglycerol;
Z is selected from: an alkyl chain; a substituted alkyl chain; a thioether; a disulfide; and an alkyl chain containing an azole ring.

12. The compound of claim 1, wherein Z is selected from: 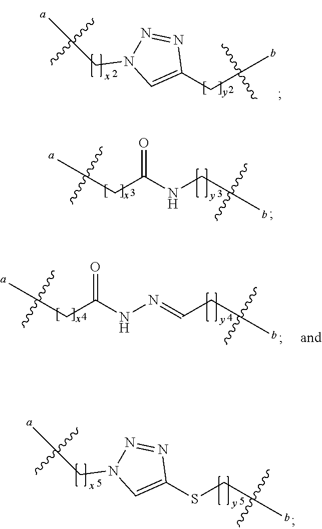
wherein,
position a connects to the sugar and b position connects to the linear polyglycerol;
x¹ is an integer between 1 and 10;
x² is an integer between 1 and 10;
y² is an integer between 1 and 10;
x³ is an integer between 1 and 10;
y³ is an integer between 1 and 10;
x⁴ is an integer between 1 and 10;
y⁴ is an integer between 1 and 10;
x⁵ is an integer between 1 and 10; and
y⁵ is an integer between 1 and 10.

13. The compound of claim 1, wherein when q1 is 1, the sialic acid is attached to the sugar by a 2,3 linkage; or 2,6-linkage; and wherein q1 is 2, the sialic acid is attached to the sugar through a α2-3, α2-6, α2-8 linkage to the sugar or another sialic acid or sialic acid derivative.

14. The compound of claim 1, wherein the sugar-sialic acid is selected from one or more of the following:

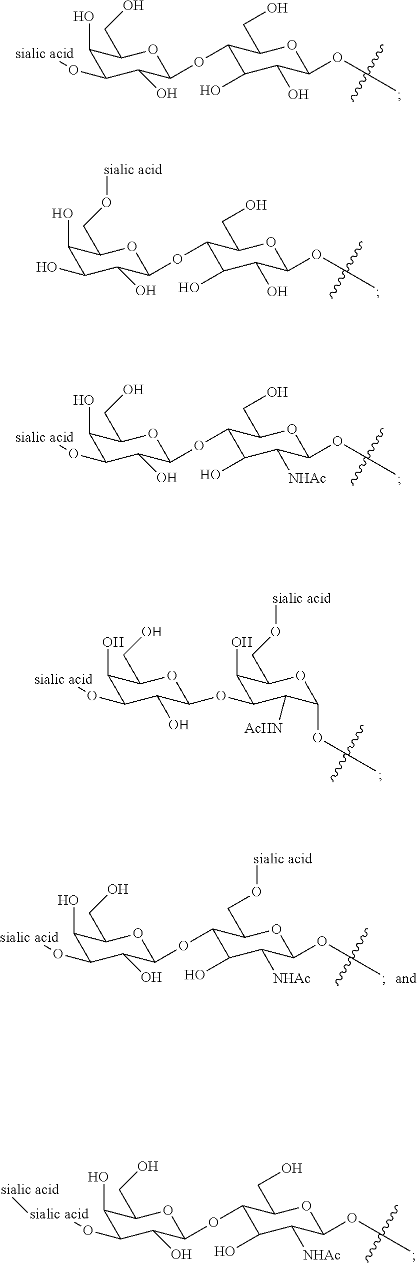

15. The compound of claim 1, wherein the sugar-sialic acid is selected from one or more of the following:

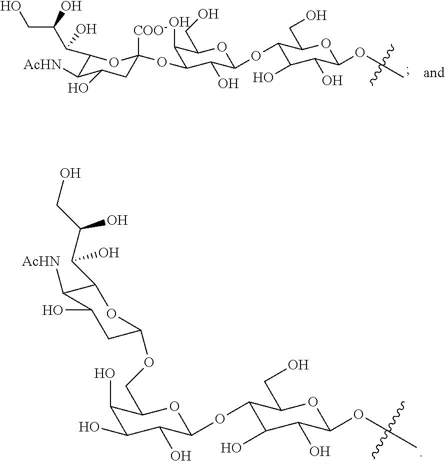

16. The compound of claim 1, wherein the compound is selected from one or more of the following:

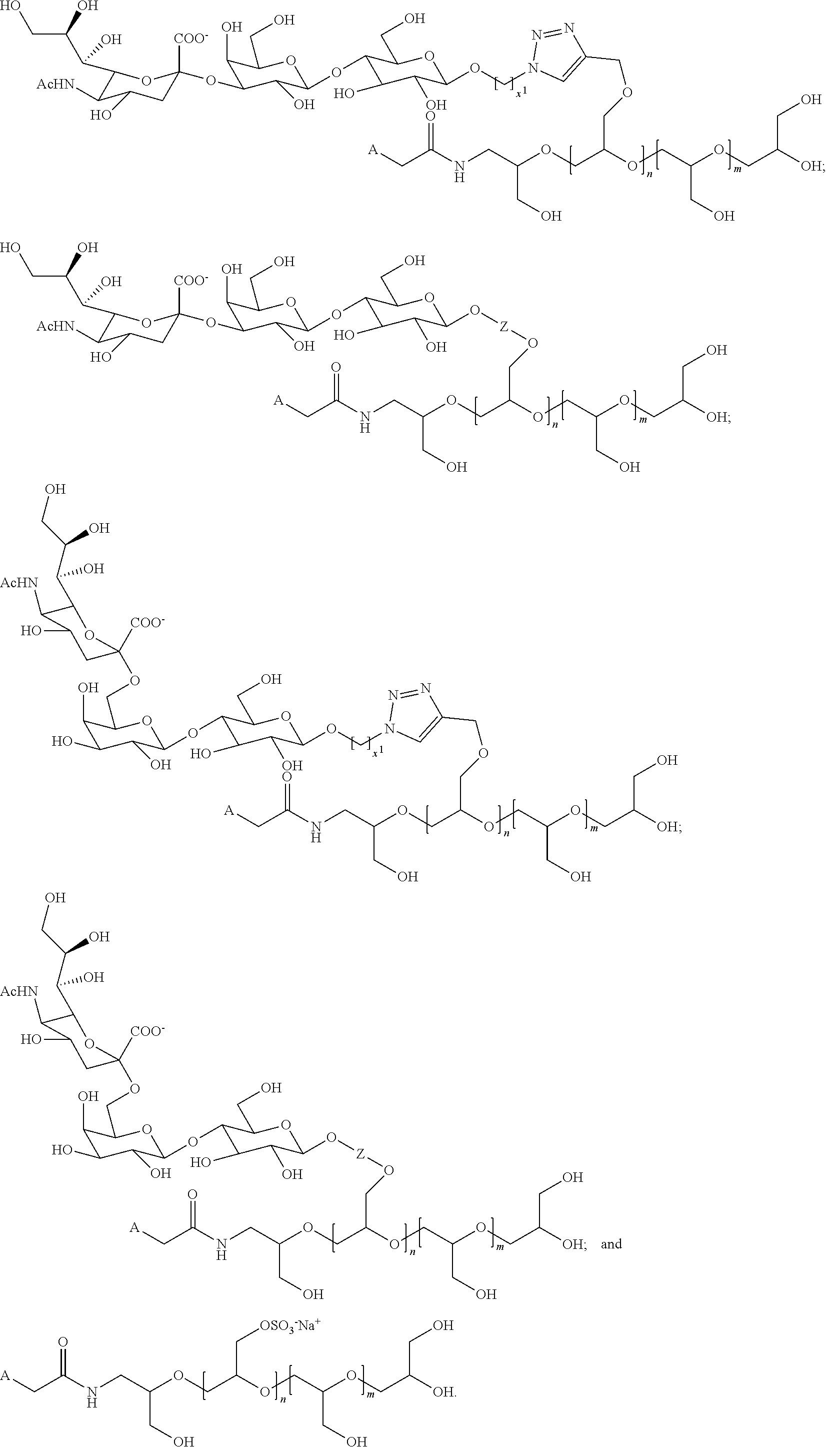

17-20. (canceled)

21. A preservation solution, the preservation solution comprising a compound of claim 1.

22. The preservation solution of claim 21, the preservation solution further comprising a cell-surface ligating enzyme.

23. (canceled)

24. The preservation solution of claim 22, the cell-surface ligating enzyme may be selected from one or more of: a transglutaminase; a sortase; an asparagine endopeptidase; a trypsin related enzyme; a butelase; and a subtiligase.

25. An ex vivo method for cell surface engineering (CSE), the method comprising:
(a) immersing an organ in a preservation solution of claim 21, wherein the organ has a glycocalyx and a vasculature; and
(b) incubating the ex vivo organ in the preservation solution to permit binding of a compound of to bind to the glycocalyx of the organ,
wherein the compound has the structure of Formula A: 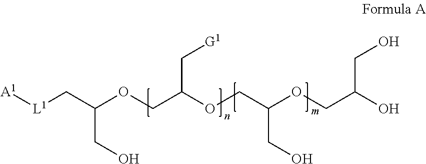
wherein,
A¹ is a peptide tag;
L¹ is a peptide linking group;
G¹ is selected from: -a linker-(a sugar)_{q1}-(a sialic acid)_{q2}; and —R¹—SO₃ group;
n is an integer between 1 and 40,000;
m is an integer between 1 and 40,000;
R¹ is selected from C, O and N;
the linker is selected from: an alkyl chain; a substituted alkyl chain; or an alkyl chain containing an azole ring;
q1 is an integer between 1 and 10; and
q2 is an integer between 1 and 10.

26. The ex vivo method of claim 25, further comprising a perfusing step, wherein the preservation solution is used to perfuse the vasculature of the organ.

27. (canceled)

28. An ex vivo method for cell surface engineering (CSE), the method comprising:
(a) immersing a cell in a preservation solution of claim 21; and
(b) incubating the ex vivo cell in the preservation solution to permit binding of a compound to bind to the surface of the cell,
Wherein the compound has the structure of Formula A: 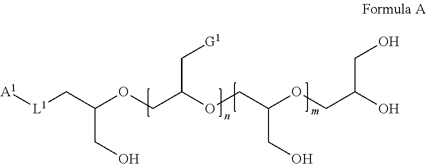
wherein,
A¹ is a peptide tag;
L¹ is a peptide linking group;
G¹ is selected from: -a linker-(a sugar)_{q1}-(a sialic acid)_{q2}; and —R¹—SO₃ group;
n is an integer between 1 and 40,000;
m is an integer between 1 and 40,000;
R¹ is selected from C, O and N;
the linker is selected from: an alkyl chain; a substituted alkyl chain; or an alkyl chain containing an azole ring;
q1 is an integer between 1 and 10; and
q2 is an integer between 1 and 10.

29-35. (canceled)

36. A compound, the compound having the structure of Formula B: 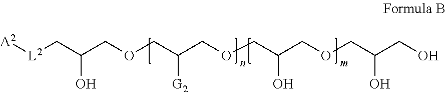
wherein,
A² is a peptide tag;
L² is a peptide linking group;
G² is selected from: -a linker-(a sugar)_{q3}-(a sialic acid)_{q4}; and a —R²—SO₃⁻ group;
n is an integer between 1 and 40,000;
m is an integer between 1 and 40,000;
R² is selected from C, O, and N;
the linker is selected from: an alkyl chain; a substituted alkyl chain; or an alkyl chain containing an azole ring;
q3 is an integer between 1 and 10; and
q4 is an integer between 1 and 10.

37-65. (canceled)

66. A method of increasing the sialic acid density on the cell or tissue or organ surface for the purpose of immunosuppression or immune conditioning post-transplantation using a compound,
wherein the compound has the structure of Formula A: 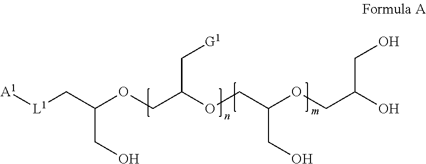
wherein,
A¹ is a peptide tag;
L¹ is a peptide linking group;
G¹ is selected from: -a linker-(a sugar)_{q1}-(a sialic acid)_{q2}; and —R¹—SO₃ group;
n is an integer between 1 and 40,000;
m is an integer between 1 and 40,000;
R¹ is selected from C, O and N;
the linker is selected from: an alkyl chain; a substituted alkyl chain; or an alkyl chain containing an azole ring;
q1 is an integer between 1 and 10; and
q2 is an integer between 1 and 10, or
wherein the compound has the structure of Formula B: 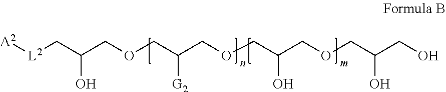
wherein,
A² is a peptide tag;
L² is a peptide linking group;
G² is selected from: -a linker-(a sugar)_{q3}-(a sialic acid)_{q4}; and a —R²—SO₃ group;
n is an integer between 1 and 40,000;
m is an integer between 1 and 40,000;
R² is selected from C, O, and N;
the linker is selected from: an alkyl chain; a substituted alkyl chain; or an alkyl chain containing an azole ring;
q3 is an integer between 1 and 10; and
q4 is an integer between 1 and 10.

67-69. (canceled)
